(19) United States
(12) Patent Application Publication
(10) Pub. No.: US 2023/0098111 A1
(43) Pub. Date: Mar. 30, 2023

(54) COMPOSITIONS AND METHODS TO TREAT NEUROLOGICAL DISEASES

(71) Applicant: University of Southern California, Los Angeles, CA (US)

(72) Inventors: Justin Ichida, Los Angeles, CA (US); Uh Jin Lee, North Chicago, IL (US); Manuel Santana, Los Angeles, CA (US)

(21) Appl. No.: 17/794,920

(22) PCT Filed: Jan. 28, 2021
(86) PCT No.: PCT/US2021/015582

§ 371 Date: Jul. 22, 2022

Related U.S. Application Data
(60) Provisional application No. 62/966,886, filed on Jan. 28, 2020.

(51) Int. Cl. C12N 15/113; A61P 25/28

(52) U.S. Cl. CPC C12N 15/1137; C12Y 207/01137; A61P 25/28; C12N 2310/11; C12N 2310/315; C12N 2310/3341; C12N 2310/3231; C12N 2310/321; C12N 2310/341; C12N 2320/30

(57) ABSTRACT

Disclosed is a method of treating a subject who has a neurological disease. The neurological disease may be associated with altered C9ORF72 protein activity. In one aspect, the method includes a step of administering an effective dose of a PIKFYVE antisense or inhibitory nucleic acid to a subject in need thereof, thereby rescuing the defects associated with altered C9ORF72 protein activity and/or inhibiting the expression of PIKFYVE gene.

### CROSS REFERENCE TO RELATED APPLICATION(S)

This application claims priority to U.S. Provisional Application No. 62/966,886, filed on Jan. 28, 2020, the disclosure of which is incorporated herein by reference in its entirety.

### TECHNICAL FIELD

The present disclosure is directed to methods to prevent and/or treat neurological diseases. Compositions useful in the herein described methods include PIKFYVE kinase inhibitors, potassium channel activators, glutamate receptor inhibitors, and endosomal and lysosomal trafficking modulators.

### INCORPORATION BY REFERENCE OF SEQUENCE LISTING

Accompanying this filing is a Sequence Listing entitled, “Sequence-Listing_ST25” created on Jan. 28, 2021 and having 35,859 bytes of data, machine formatted on IBM-PC, MS-Windows operating system. The sequence listing is hereby incorporated by reference in its entirety for all purposes.

### BACKGROUND

Many neurological diseases are progressive and may result in a wide range of symptoms such as weakness in the extremities, slurring of speech, vision abnormalities, difficulty breathing, difficulty swallowing, dementia, impaired balance, loss of memory, unsteady gait, muscle twitching, depression, anxiety, and mood swings. Some of these diseases are inherited, but the etiology of many cases is unknown. Indeed, the basis for many of these diseases may be due to environmental, toxic, or viral factors, as well as genetic predisposition, or a combination thereof. A GGGGCC repeat expansion ((GGGGCC)ₙ) in C9ORF72 is a cause of neurological diseases, including amyotrophic lateral sclerosis (ALS) and frontotemporal dementia (FTD), accounting for about 10% of each worldwide. There is no cure for many neurological diseases and treatment options are limited, thus there is a need in the art for methods of treating the diseases.

### SUMMARY

The disclosure provides an oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 1-136. In one embodiment, the nucleobase sequence of the oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to any one of SEQ ID NOs: 1-136. In another embodiment, the oligonucleotide consists of a single-stranded modified oligonucleotide. In yet another embodiment of any of the foregoing, the oligonucleotide is complementary to the PIKFYVE mRNA sequence of FIG. 18 (T can be U or vice-a-versa where appropriate). In yet another embodiment of any of the foregoing embodiments, at least one internucleoside linkage is a modified internucleoside linkage. In a further embodiment, at least one modified internucleoside linkage is a phosphorothioate internucleoside linkage. In yet another embodiment, each modified internucleoside linkage is a phosphorothioate internucleoside linkage. In still another embodiment of any of the foregoing embodiments, at least one internucleoside linkage is a phosphodiester internucleoside linkage. In still further embodiments, at least one internucleoside linkage is a phosphorothioate linkage and at least one internucleoside linkage is a phosphodiester linkage. In yet further embodiments of the foregoing, at least one nucleoside comprises a modified nucleobase. In a further embodiment, the modified nucleobase is a 5-methylcytosine. In yet another embodiment of any of the foregoing at least one nucleoside of the modified oligonucleotide comprises a modified sugar. In a further embodiment, the at least one modified sugar is a bicyclic sugar. In still a further embodiment, the bicyclic sugar comprises a 4′-CH(R)—O-2′ bridge wherein R is, independently, H, C₁₋₁₂ alkyl, or a protecting group. In yet a further embodiment, R is methyl. In another embodiment, R is H. In another embodiment, at least one modified sugar comprises a 2′-O-methoxyethyl group. In still another embodiment of any of the foregoing embodiments, the oligonucleotide comprises a gap segment consisting of 8 to 12 linked deoxynucleosides; a 5′ wing segment consisting of 3 to 5 linked nucleosides; and a 3′ wing segment consisting of 3 to 5 linked nucleosides; wherein the gap segment is positioned between the 5′ wing segment and the 3′ wing segment and wherein a nucleoside of each wing segment comprises a modified sugar. In a further embodiment, each nucleoside of each wing segment comprises a modified sugar. In another embodiment of any of the foregoing, the oligonucleotide consists of 20 linked nucleosides.

The disclosure also provides an antisense oligonucleotide comprising a sequence and/or structure as set forth in Table 1 or Table 2, wherein the sequence or structure is at least 8-22 nucleotide in length and sequences that are at least 98-99% identical thereto and which inhibit the expression of PIKFYVE gene.

The disclosure also provides a method of treating a subject having a neurological disease, the method including the step of administering to the subject an effective dose of a PIKFYVE antisense molecule, vector expressing a PIKFYVE antisense molecule, a PIKFYVE inhibitory nucleic acid and/or a vector expressing a PIKFYVE inhibitory nucleic acid. In one embodiment, the PIKFYVE antisense molecule is an oligonucleotide as described in any of the embodiments herein. In another embodiment, the neurological disease is amyotrophic lateral sclerosis (familial or sporadic). In yet another embodiment, the neurological disease is frontotemporal dementia. In still another embodiment, the neurological disease is associated with aberrant endosomal trafficking. In another embodiment, the neurological disease is associated with aberrant lysosomal trafficking. In a further embodiment, the subject is haploinsufficient for the C9ORF72 gene. In yet a further embodiment, the haploinsufficiency results in a 50% or greater reduction in C9ORF72 protein activity. In another embodiment, the C9ORF72 gene product comprises a dipeptide repeat resulting from the (GGGGCC)ₙ expansion. In a further embodiment, the dipeptide repeat is cytotoxic. In another embodiment, the neurological disease is associated with neuronal hyperexcitability.

The disclosure also provides a modified oligonucleotide, wherein the modified oligonucleotide is a gapmer consisting of a 5′ wing segment, a central gap segment, and a 3′ wing segment, wherein: the 5′ wing segment consists of 3-5 modified nucleosides, the central gap segment consists of 8-12 nucleosides, and the 3′ wing segment consists of 3-5 modified nucleosides; wherein the modified oligonucleotide has the nucleobase sequence of any one of SEQ ID NOs: 1-136. In one embodiment, the 3′ and/or 5′ wing segments comprise modified nucleobases selected from the group consisting of 2′-OMe, 2′-MOE, LNA, DNA and any combination thereof.

In a particular embodiment, the disclosure provides for a single stranded antisense oligonucleotide (ASO) that suppresses the expression of a PIKFYVE encoded by the sequence of SEQ ID NO:137, wherein the ASO comprises 12 to 50 linked nucleosides. In a further embodiment, the ASO has 18 to 20 linked nucleosides. In another embodiment or a further embodiment of any of the foregoing embodiments, the at least one internucleoside linkage is a modified internucleoside linkage. In another embodiment or a further embodiment of any of the foregoing embodiments, at least one modified internucleoside linkage is a phosphorothioate internucleoside linkage. In another embodiment or a further embodiment of any of the foregoing embodiments, each modified internucleoside linkage is a phosphorothioate internucleoside linkage. In another embodiment or a further embodiment of any of the foregoing embodiments, at least one internucleoside linkage is a phosphodiester internucleoside linkage. In another embodiment or a further embodiment of any of the foregoing embodiments, at least one internucleoside linkage is a phosphorothioate linkage and at least one internucleoside linkage is a phosphodiester linkage. In another embodiment or a further embodiment of any of the foregoing embodiments, at least one nucleoside comprises a modified nucleobase. In another embodiment or a further embodiment of any of the foregoing embodiments, the modified nucleobase is a 5-methylcytosine. In another embodiment or a further embodiment of any of the foregoing embodiments, at least one nucleoside of the ASO comprises a modified sugar moiety. In another embodiment or a further embodiment of any of the foregoing embodiments, at least one modified sugar moiety is a bicyclic sugar moiety. In another embodiment or a further embodiment of any of the foregoing embodiments, the bicyclic sugar moiety comprises a 4′-CH(R)—O-2′ bridge wherein R is, independently, H, C₁₋₁₂ alkyl, or a protecting group. In another embodiment or a further embodiment of any of the foregoing embodiments, R is methyl. In another embodiment or a further embodiment of any of the foregoing embodiments, R is H. In another embodiment or a further embodiment of any of the foregoing embodiments, the modified sugar moiety comprises a 2′-O-methoxyethyl group. In another embodiment or a further embodiment of any of the foregoing embodiments, the ASO is a gapmer. In another embodiment or a further embodiment of any of the foregoing embodiments, the ASO comprises: a gap segment consisting of 8 to 12 linked deoxynucleosides; a 5′ wing segment consisting of 3 to 5 linked nucleosides; and a 3′ wing segment consisting of 3 to 5 linked nucleosides; wherein the gap segment is positioned between the 5′ wing segment and the 3′ wing segment and wherein a nucleoside of each wing segment comprises a modified sugar moiety. In another embodiment or a further embodiment of any of the foregoing embodiments, each nucleoside of each wing segment comprises a modified sugar moiety. In another embodiment or a further embodiment of any of the foregoing embodiments, the nucleosides making up each wing segment comprises at least two different modified sugar moieties. In another embodiment or a further embodiment of any of the foregoing embodiments, the nucleosides making up each wing segment comprises the same modified sugar moiety. In another embodiment or a further embodiment of any of the foregoing embodiments, the modified sugar moiety comprises a 2′-O-methoxyethyl group. In another embodiment or a further embodiment of any of the foregoing embodiments, the ASO has a nucleobase sequence that comprises at least 15 consecutive nucleobases of any of the nucleobase sequences of SEQ ID NOs: 1-136. In another embodiment or a further embodiment of any of the foregoing embodiments, the ASO has a nucleobase sequence of any one of SEQ ID NOs:1-136. In another embodiment or a further embodiment of any of the foregoing embodiments, the ASO has a nucleobase sequence of any one of SEQ ID NOs:46, 49, 56, 60, 62, 64, 65, 70, 71, 73 and 105. In another embodiment or a further embodiment of any of the foregoing embodiments, the ASO is a gapmer consisting of a 5′ wing segment, a central gap segment, and a 3′ wing segment, wherein: the 5′ wing segment consists of 3-5 modified nucleosides, the central gap segment consists of 8-12 nucleosides, and the 3′ wing segment consists of 3-5 modified nucleosides; wherein a modified nucleoside of each wing segment comprises a modified sugar moiety; and wherein the ASO has the nucleobase sequence of any one of SEQ ID NOs: 1-136. In another embodiment or a further embodiment of any of the foregoing embodiments, the ASO has a nucleobase sequence of any one of SEQ ID NOs:46, 49, 56, 60, 62, 64, 65, 70, 71, 73 and 105. In another embodiment or a further embodiment of any of the foregoing embodiments each modified nucleoside of each wing segment comprises a modified sugar moiety. In another embodiment or a further embodiment of any of the foregoing embodiments, the modified nucleosides making up each wing segment comprises at least two different modified sugar moieties. In another embodiment or a further embodiment of any of the foregoing embodiments, the modified nucleosides making up each wing segment comprises the same modified sugar moiety. In another embodiment or a further embodiment of any of the foregoing embodiments, the modified sugar moiety comprises a 2′-O-methoxyethyl group.

In a certain embodiment, the disclosure also provides a pharmaceutical composition comprising the ASO of any one of the preceding aspects, and a pharmaceutically acceptable carrier, diluent and/or excipient. In a further embodiment, the pharmaceutical composition is formulated for parenteral delivery. In another embodiment or a further embodiment of any of the foregoing embodiments, the pharmaceutical composition is formulated for intracerebroventricular injection.

In a particular embodiment, the disclosure further provides a method of treating a subject having a neurological or neurodegenerative disease in need of treatment thereof, comprising: administering a therapeutically effective amount of a pharmaceutical composition disclosed herein, or a therapeutically effective amount of an ASO disclosed herein. In a further embodiment, the subject is haploinsufficient for the C9ORF72 gene. In yet a further embodiment, the haploinsufficiency results in a 50% or greater reduction in C9ORF72 protein activity. In yet a further embodiment, the C9ORF72 gene product comprises a dipeptide repeat resulting from the (GGGGCC)ₙ expansion. In yet a further embodiment, the dipeptide repeat is cytotoxic. In another embodiment or a further embodiment of any of the foregoing embodiments, the neurological disease is associated with neuronal hyperexcitability. In another embodiment or a further embodiment of any of the foregoing embodiments, the neurological disease is associated with aberrant endosomal trafficking. In another embodiment or a further embodiment of any of the foregoing embodiments, the neurological disease is associated with aberrant lysosomal trafficking. In another embodiment or a further embodiment of any of the foregoing embodiments, the neurological disease is selected from the group consisting of familial and sporadic amyotrophic lateral sclerosis (ALS), familial and sporadic frontotemporal dementia (FTD), progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubro-pallidoluysian atrophy, spinocerebellar ataxias, and Creutzfeldt-Jakob disease. In another embodiment or a further embodiment of any of the foregoing embodiments, the neurological disease is ALS. In another embodiment or a further embodiment of any of the foregoing embodiments, the neurological disease is FTD.

The disclosure also provides pharmaceutical compositions comprising the oligonucleotides or modified oligonucleotides of the disclosure and a pharmaceutically acceptable diluent or carrier.

### BRIEF DESCRIPTION OF THE DRAWINGS

FIG. 1 shows PIKFYVE ASOs rescue C9-ALS iMN survival.

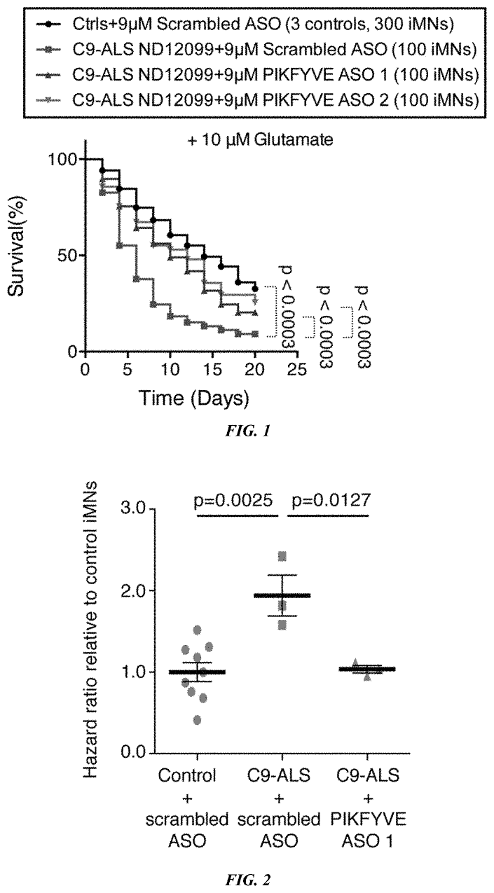

FIG. 2 shows Hazard ratios of C9-ALS iMNs (relative to control iMNs) when treated with a scrambled or PIKFYVE ASO.

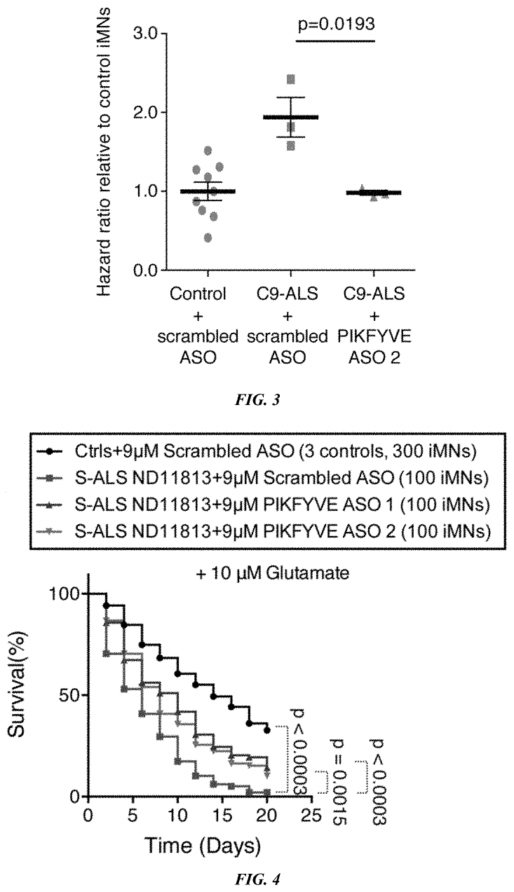

FIG. 3 shows Hazard ratios of C9-ALS iMNs (relative to control iMNs) when treated with a scrambled or PIKFYVE ASO.

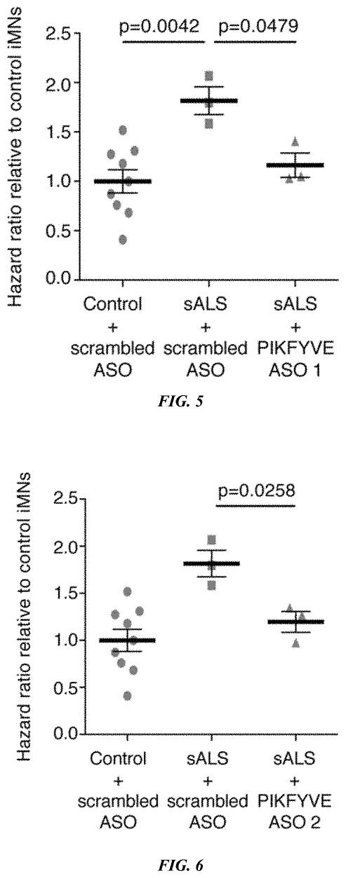

FIG. 4 shows PIKFYVE ASOs rescue sporadic ALS iMN survival from patient ND11813.

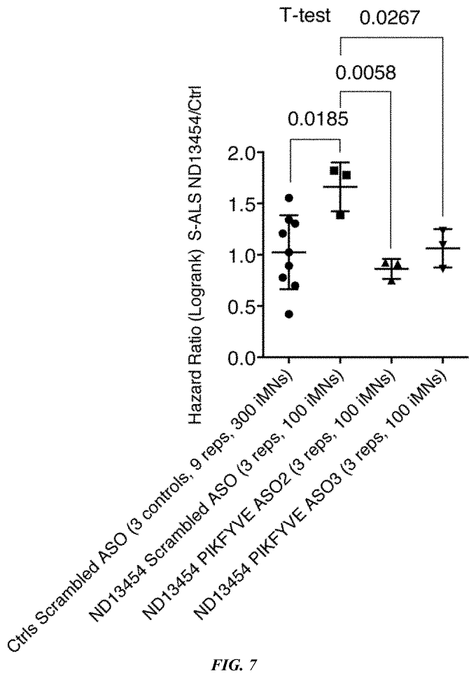

FIG. 5 shows Hazard ratios of sporadic ALS iMNs (relative to control iMNs) when treated with a scrambled or PIKFYVE ASO.

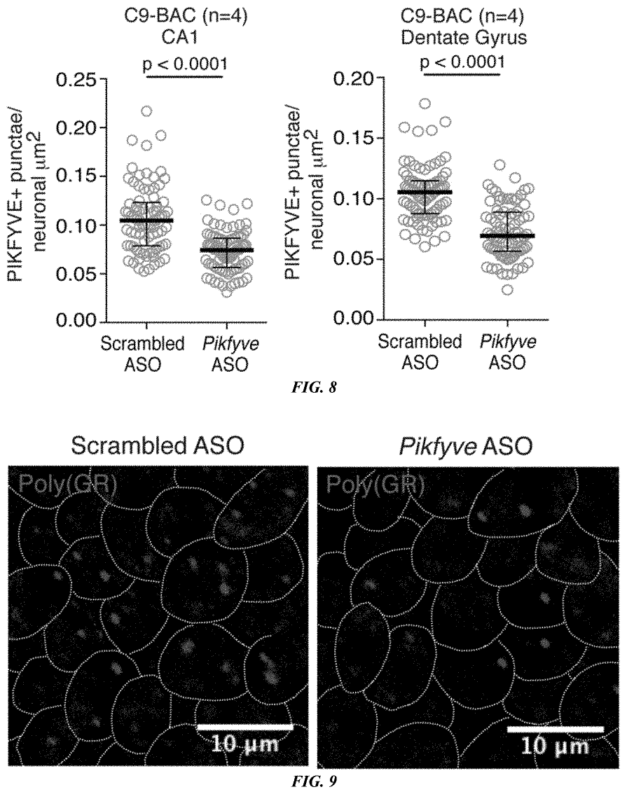

FIG. 6 shows Hazard ratios of sporadic ALS iMNs (relative to control iMNs) when treated with a scrambled or PIKFYVE ASO.

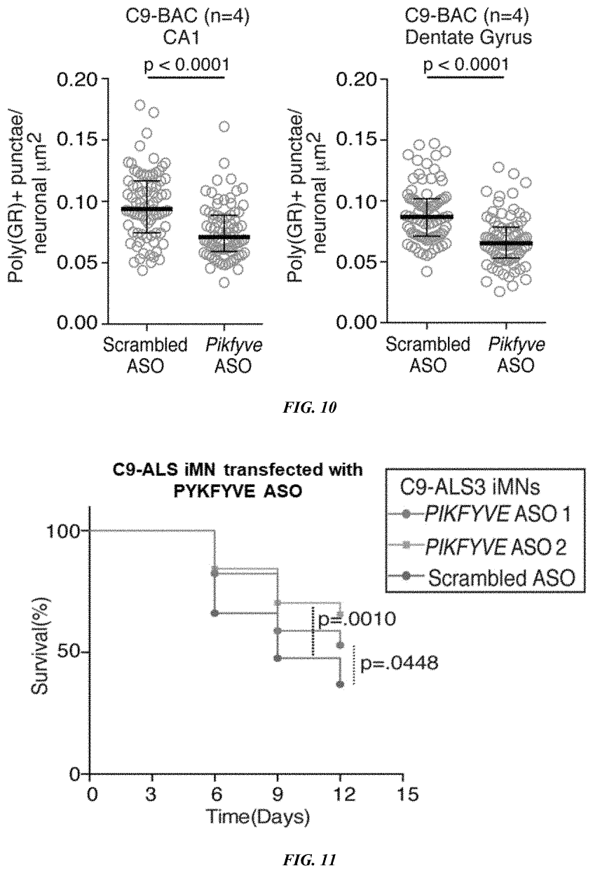

FIG. 7 shows PIKFYVE ASOs rescue sporadic ALS iMN survival from Patient ND13454.

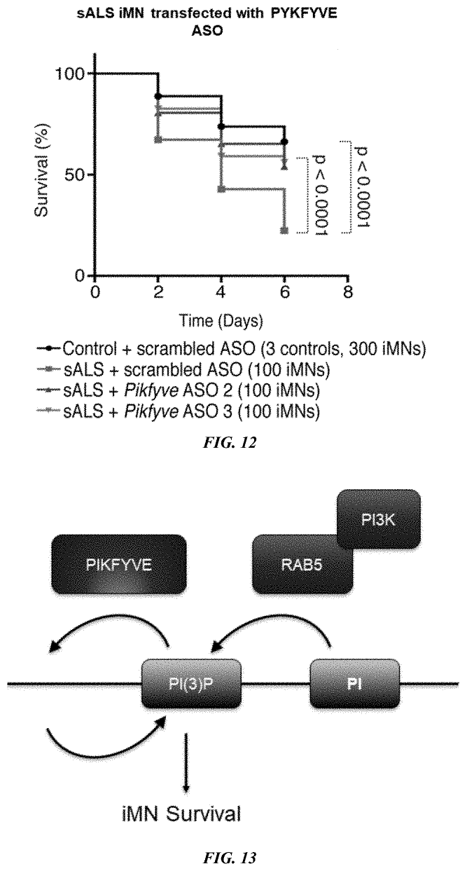

FIG. 8 shows results from ASO injected into the hippocampus 12 days prior to fixation and immunohistochemistry quantification of PIKFYVE.

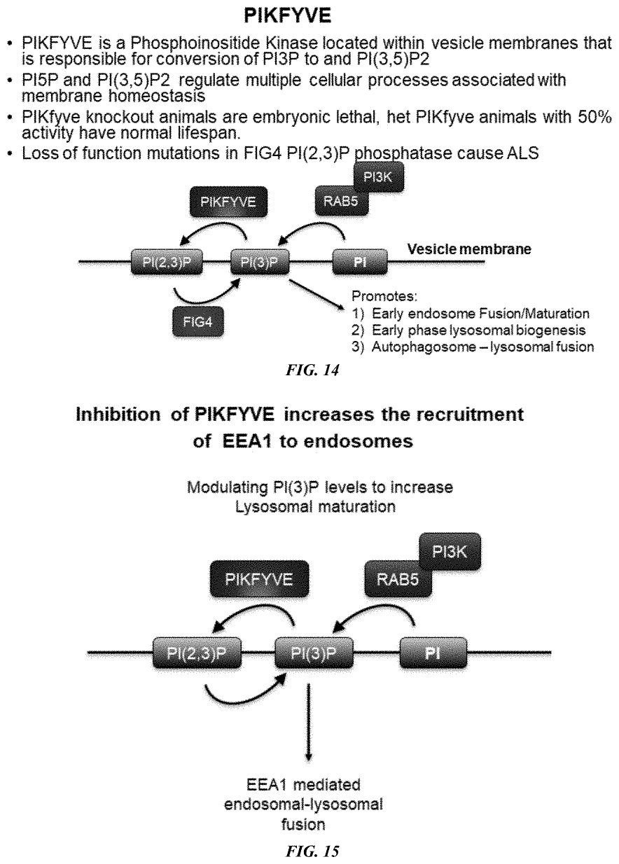

FIG. 9 shows results following ASO injected into the hippocampus 12 days prior to fixation and IHC quantification of DRPs. DRP aggregates (Poly(GR) red) accumulate in the cells (white outline) of C9-BAC mice.

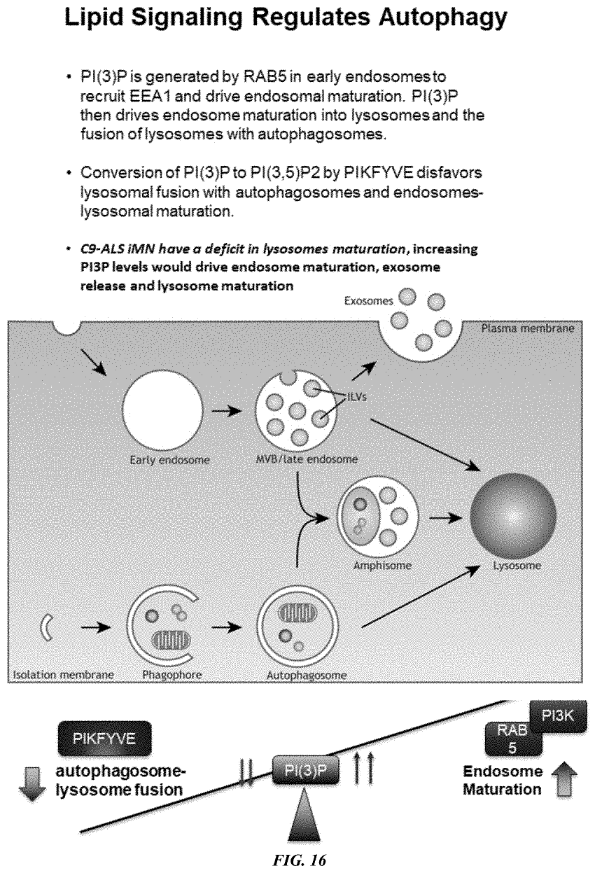

FIG. 10 shows results C9-BAC animals treated with Pikfyve ASO have significantly less DRP aggregates in the hippocampus when compared to scrambled controls.

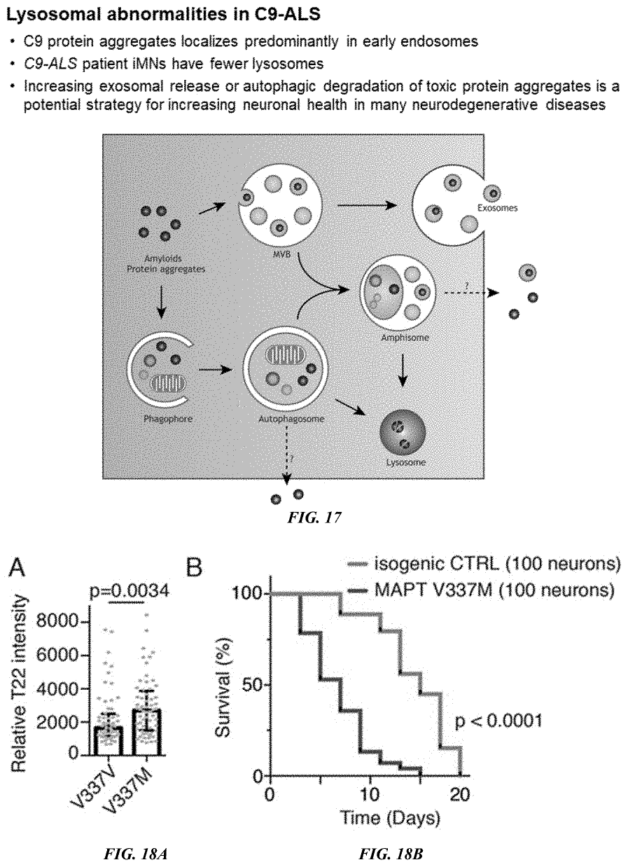

FIG. 11 shows a survival curve using ASO 1 and 2 (Table 2).

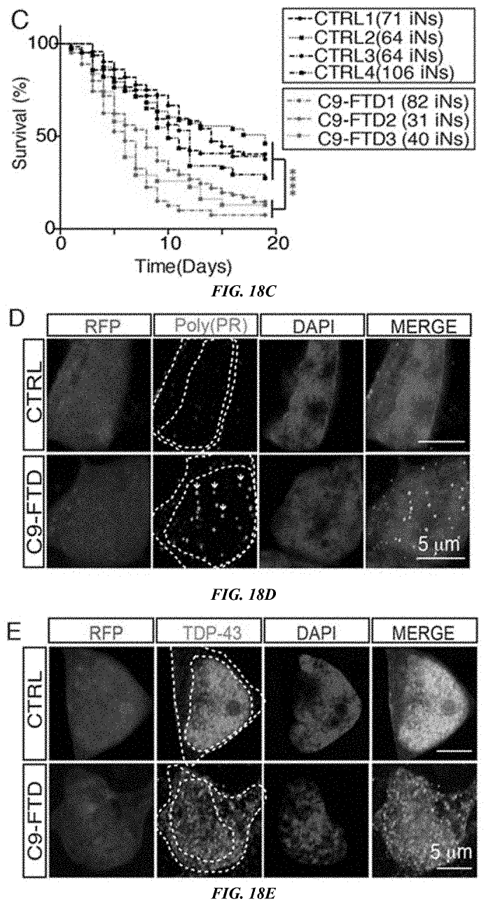

FIG. 12 shows a survival curve using ASO 2 and 3 (Table 2).

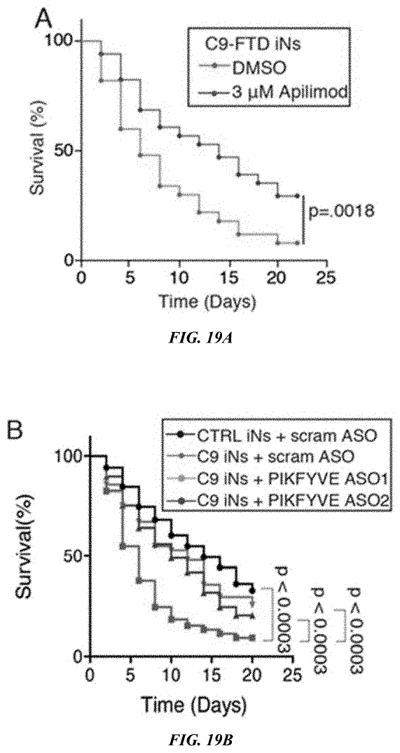

FIG. 13 provides a schematic showing the biological interactions and data using apilimod and ASO.

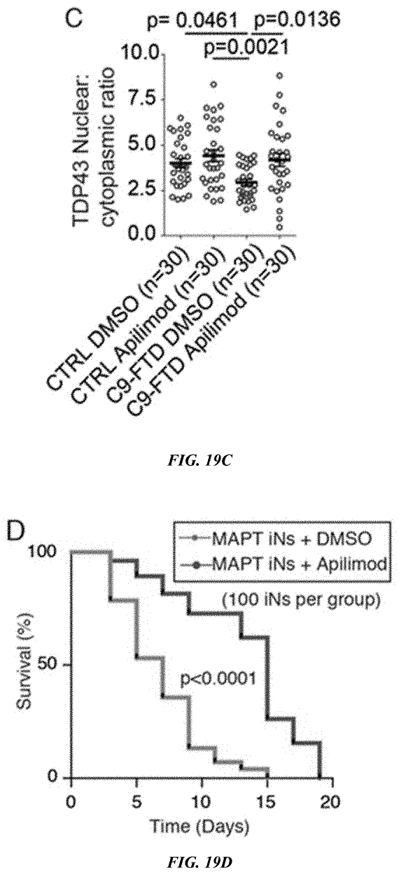

FIG. 14 is a schematic explaining the biological interactions of PIKFYVE and pathways.

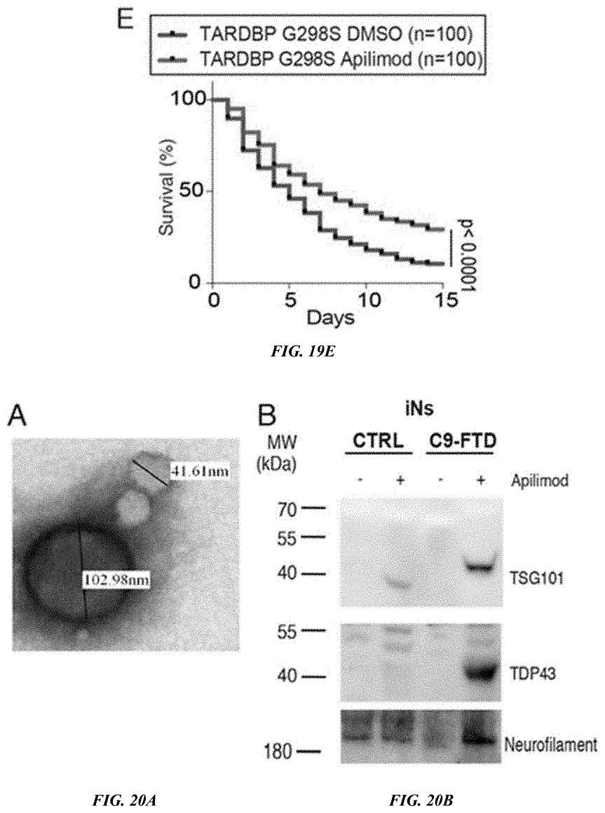

FIG. 15 shows that inhibition of PIKFYVE increase the recruitment of EEA1 to endosomes.

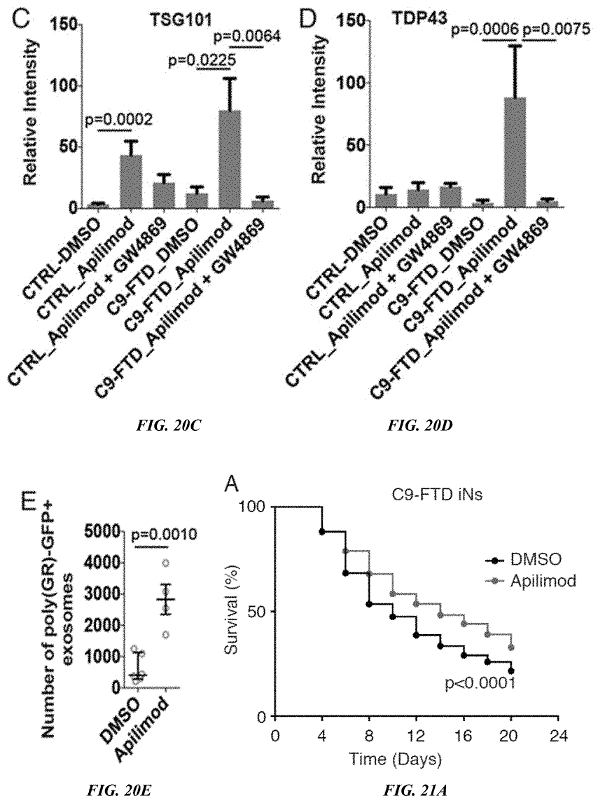

FIG. 16 is another explanation of the role of PIKFYVE and pathway effects.

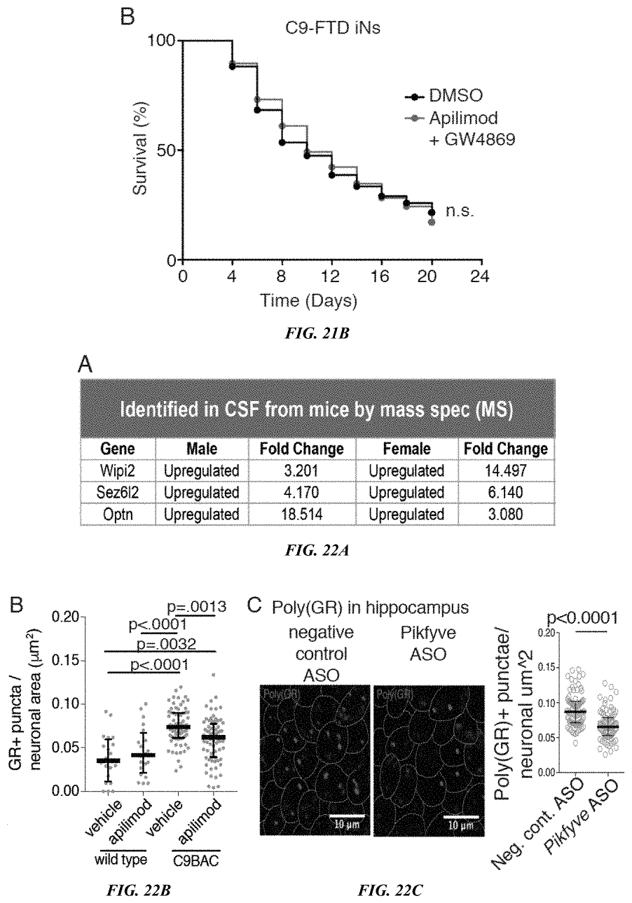

FIG. 17 depicts the lysosomal abnormalities in C9-ALS.

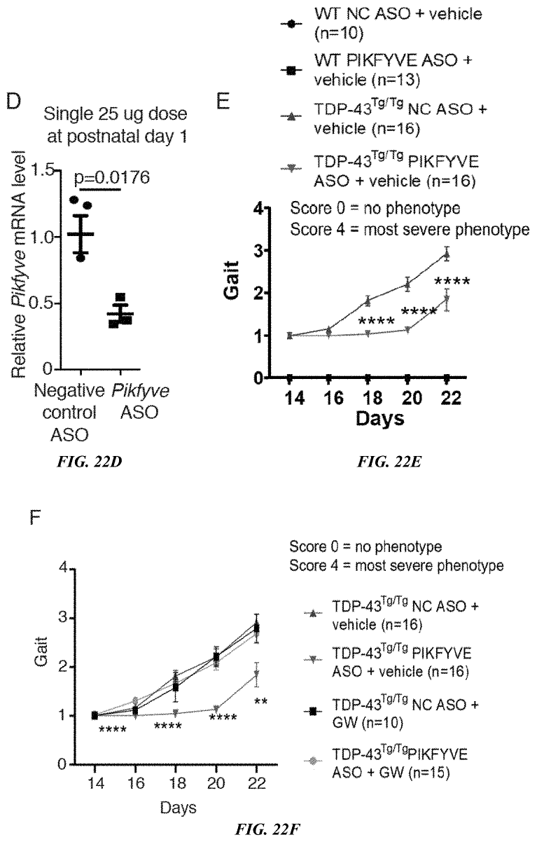

Other aspects of the invention will become apparent by consideration of the detailed description and accompanying drawings.

FIG. 18A-E provides for the establishment of iN FTD models. (A) Tau oligomer (T22) immunostaining intensity in MAPT V337M or isogenic control iPSC neurons. Each data point represents one neuron. Data derived from 3 independent replicates. Median+/−interquartile range. Mann-Whitney test. (B) Survival of control or MAPT V337M FTD iNs. Log-rank test. Neurons quantified from 3 independent replicates. (C) Survival of control or C9ORF72 FTD/ALS iNs. Log-rank test. Neurons quantified from 3 independent replicates. (D,E) Poly(PR) DPRs (D) and TDP-43 localization (E) in control or C9ORF72 FTD/ALS iNs. White dotted lines outline the nucleus and cell body.

FIG. 19A-E demonstrates PIKFYVE inhibition rescues FTD iN survival. (A) Survival of C9ORF72 iNs with DMSO or apilimod. Log-rank test, 100 iNs per group. (B) Survival of control and C9ORF72 iNs treated with scrambled or PIKFYVE ASOs. Log-rank test, 100 iNs per group. (C) Nuclear:cytoplasmic ratio of TDP-43 in control or C9ORF72 iNs treated with DMSO or apilimod. n=30 iNs per group. Mean+/−s.e.m. Kruskal-Wallis Test. (D, E) Survival of MAPT FTD iNs (D) or TARDBP iNs (E) in DMSO or apilimod. Log-rank test, 100 iNs per group. iNs quantified from 3 independent iN conversions per line.

FIG. 20A-E shows that PIKFYVE inhibition induces exosome secretion in iNs. (A) Electron microsome image of exosomes from apilimod-treated C9-FTD iNs. (B) Western blot of TSG101, TDP-43, and Neurofilament heavy chain levels in the exosome fraction secreted from control or C9-FTD iNs upon apilimod treatment. (C,D) Quantification of the exosome marker TSG101 (C) or TDP-43 (D) in the exosome fraction of control and C9-FTD iNs treated with DMSO, apilimod, or apilimod+GW4869. Kruskal-Wallis test for B, C. N=3 biological replicates per condition, one line per genotype. (E) Number of poly(GR)-GFP+ exosomes secreted from iNs expressing exogenous poly(GR)-GFP upon DMSO or apilimod treatment. N=4 biological replicates. Exosomes were labeled with CFSE-red dye to enable detection by FACS. Unpaired t-test.

FIG. 21A-B shows that PIKFYVE inhibition rescues FTD iN survival through secretory autophagy. (A, B) Survival of C9-FTD iNs treated with DMSO and Apilimod (A) or Apilimod+GW4869 (exocytosis blocker) (B). Log-rank test. 100 iNs per group. iNs quantified from 3 independent replicates per group.

FIG. 22A-F demonstrates that Pikfyve suppression induces secretory autophagy and rescues motor deficits in TDP-43 ALS/FTD mice. (A) Mass spectrometry analysis of Optineurin (OPTN) levels in the cerebrospinal fluide (CSF) of mice 2 hours after direct intracerebroventral injection of a vehicle control or apilimod. n=3 mice per group. The ratio of Optineurin in the CSF of apilimod:vehicle-injected mice is shown. (B) Poly(GR)+ DPR punctae in wild-type or C9ORF72 mice treated with intracerebroventricular injection of apilimod for 48 hours. Mean+/−s.e.m. Data points are individual neurons taken across 3 mice per group. One-way ANOVA. (C) Poly(GR)+ punctae in the hippocampus of adult C9ORF72-BAC mice injected with a negative control (Neg. cont.) or Pikfyve ASO for 7 days. Unpaired t-test. (D) Pikfyve qRT-PCR of whole brain. n=3 mice per group at postnatal day 7. Unpaired t-test. (E) Gait impairment scoring in wild-type (WT) and TDP-43 homozygous mice treated with a negative control (NC) ASO or Pikfyve ASO at postnatal day 1. Mean+/−s.e.m. Unpaired t-test at each time point between the negative control and Pikfyve ASO conditions for TDP-43 mice. A score of 0 indicates no phenotype and 4 indicates the most severe phenotype. (F) Vehicle or GW4869 were delivered by intraperitoneal injection every 48 h starting from day 6 in negative control and Pikfyve ASO-treated mice. Mean+/−s.e.m. One-way ANOVA at each time point between the four conditions. *p<0.05, **p<0.01, ***p<0.001, ****p<0.0001.

FIG. 23A-C demonstrates C9ORF72+/−; C9ORF72-BAC mice exhibit behavioral dysfunction. Comparisons of C9+/− and C9+/−; C9-BAC mice in (A). Latency to fall in a hanging wire test, (B) Total distance travelled in 5 minutes in an open field test, and (C) Frequency in the center over 5 minutes in an open field test. N=8 C9+/− mice and 14 C9+/−; C9-BAC mice. Mean+/−s.e.m. Unpaired t-test.

FIG. 24A-G demonstrates Pikfyve suppression rescues TDP-43 pathology, neurodegeneration, and survival in TDP-43 mice. (A, B) Images (A) and quantification (B) of phosphorylated TDP-43 (pTDP43)+ punctae in spinal motor neurons of day 21 wild-type or TDP-43 mutant mice treated with a negative control (NC ASO) or Pikfyve ASO. Each data point represents one mouse, mean+/−s.e.m., one-way ANOVA. In (A), each arrow marks a pTDP-43+ puncta. Dotted lines outline the nucleus and cell body for each neuron. (C, D) Images (C) and quantification (D) of nuclear and cytoplasmic TDP-43 intensity in spinal motor neurons of day 21 wild-type or TDP-43 mutant mice treated with a negative control (NC ASO) or Pikfyve ASO. Each data point represents one mouse, mean+/−s.e.m., one-way ANOVA. In (C), dotted lines outline the nucleus and cell body for each neuron. (E, F) Images (E) and quantification (F) of the average number of Neurotrace (Nissl)+ spinal motor neurons per ventral horn per mouse. Each data point represents one mouse, mean, one-way ANOVA. In (E), orange dotted lines outline the ventral horn area quantified and white dotted lines outline the spinal cord. (G) Survival of TDP-43 mutant mice treated with a negative control (NC ASO) or Pikfyve ASO. Log-rank test. *p<0.05, **p<0.01, ***p<0.001, ****p<0.0001.

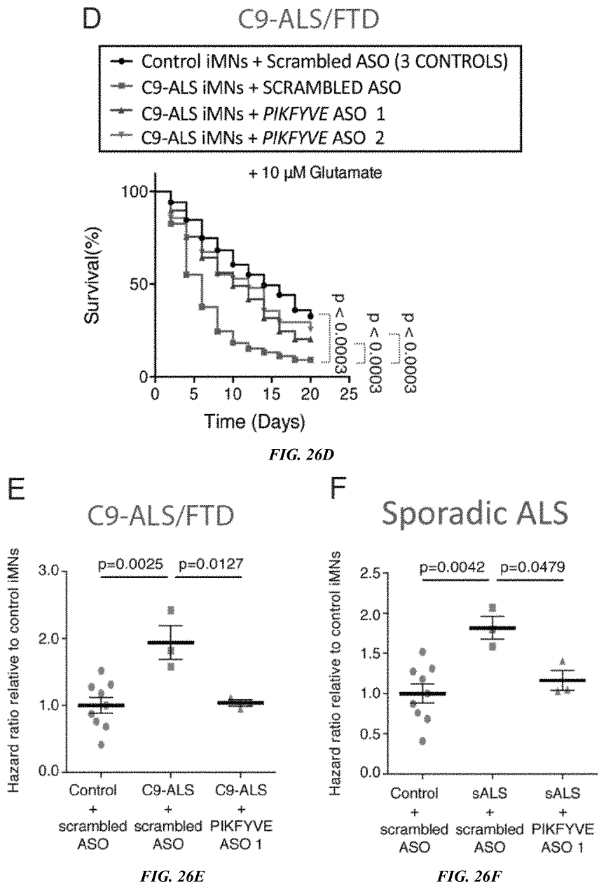

FIG. 25A-E demonstrates C9ORF72 and sporadic ALS iMNs display ALS phenotypes. (A) Survival of control (CTRL) and C9ORF72 ALS/FTD patient (C9-ALS) iMNs with a 12-hour pulse treatment of excess glutamate shown for each individual line separately. iMNs quantified from 3 independent iMN conversions per line. (B-D) Immunofluorescence analysis of total TDP-43 (B) and quantification of the ratio of nuclear to cytoplasmic TDP-43 in control, C9-ALS (C), or sporadic ALS (D) iMNs. MNs from 2 controls and 2 C9-ALS patients (C) or 4 sporadic ALS patients (D) were quantified. n=30 (controls), 30 (C9-ALS/FTD), or 36 (sporadic) iMNs per line per condition from 2 biologically independent iMN conversions of 2 control, 2 C9-ALS/FTD, or 4 sporadic ALS lines were quantified. Each gray circle represents a single iMN. Median±interquartile range. Unpaired Mann-Whitney test. Scale bars: 5 μm. Dotted lines outline the nucleus and cell body. (E) Survival of control and sporadic iMNs with glutamate treatment. iMNs quantified from 3 independent iMN conversions per line. For iMN survival experiments, significance was measured by 2-sided log-rank test using the entire survival time course.

FIG. 26A-K demonstrates PIKFYVE inhibition rescues C9ORF72 ALS/FTD and sporadic ALS iMN survival and proteostasis. (A) Results of a drug screen looking at neuron survival with iMNs from ALS Patients. (B) Survival of C9-ALS/FTD iMNs with DMSO or apilimod. (C) Survival of sporadic ALS induced motor neurons (iMNs) with DMSO or apilimod. (D) Survival of C9-ALS/FTD iMNs with a scrambled ASO or PIKFYVE ASOs, 100 iMNs per group. (E) Hazard ratio of the C9-ALS/FTD survival experiment in B, showing the likelihood of iMN death in each condition. (F) Hazard ratio of sporadic ALS iMN survival with a scrambled or PIKFYVE ASO. (G, H) Poly(GR) levels in the hippocampus of C9ORF72-BAC mice injected with a scrambled or Pikfyve ASO for 7 days. Unpaired t-test. (I, J) Nuclear:cytoplasmic TDP-43 ratio in C9-ALS/FTD iMNs treated with DMSO or apilimod. While lines outline nuclei and cell bodies. One-way ANOVA. (K) Nuclear:cytoplasmic TDP-43 ratio in sporadic ALS iMNs treated with DMSO or apilimod. Unpaired t-test. iMN survival experiments analyzed by log-rank test.

FIG. 27A-D demonstrates PIKFYVE inhibition induces exosome secretion in iMNs. (A) Western blot of TSG101, TDP-43, and Neurofilament heavy chain levels in the exosome fraction secreted from control or C9-ALS/FTD iMNs upon apilimod treatment. (B) Quantification of the exosome marker TSG101 in the exosome fraction of control and C9-ALS/FTD iMNs treated with DMSO, apilimod, or apilimod+GW4869. (C) Quantification of TDP-43 in the exosome fraction of control and C9-ALS/FTD iMNs treated with DMSO, apilimod, or apilimod+GW4869. Kruskal-Wallis test for B, C. N=3 biological replicates per condition, one line per genotype. (D) Number of poly(GR)-GFP+ exosomes secreted from iMNs expressing exogenous poly(GR)-GFP upon DMSO or apilimod treatment. N=4 biological replicates. Exosomes were labeled with CFSE-red dye to enable detection by FACS. Unpaired t-test.

FIG. 28A-D shows PIKFYVE inhibition rescues SOD1 ALS, TDP-43 ALS, and MAPT FTD patient neuron survival. (A) Survival of control or SOD1 A4V iMNs with DMSO or 3 uM apilimod. (B) Survival of TDP-43 G298S iMNs with DMSO or 3 uM apilimod. (C) Survival of MAPT V337M or isogenic control iNs. (D) Survival of MAPT V337M iNs with DMSO or 3 uM apilimod. iMNs quantified from 3 independent iMN conversions per line. Significance measured by log-rank test using the entire survival time course.

FIG. 29A-D presents Apilimod pharmacokinetics and PIKFYVE ASO therapeutic window. (A) CSF concentration of apilimod over time after oral dosing at 100 mg/kg. Required concentration to be active is about 100 ng/ml. (B-D) Survival of C9ORF72 ALS/FTD iMNs with 1-10 uM negative control or PIKFYVE ASO. All doses rescue iMN survival. 100 iMNs per condition quantified from 3 independent iMN conversions per line. Significance measured by log-rank test using the entire survival time course. HR=hazard ratio comparing the PIKFYVE ASO-treated vs negative control-treated iMNs.

FIG. 30A-C shown that Pikfyve RNAi rescues motor function in TDP43 Drosophila larvae. (A) Larval turning time in TDP-43-overexpressing larvae with and without Pikfyve RNAi. (B) Larval turning time in SOD1-overexpressing and (C) C9ORF72 repeat expansion-expressing larvae. Kruskal-Wallis test.

FIG. 31A-D shows that apilimod suppresses NMJ degeneration in a Drosophila ALS model. Apilimod (30 μM) was fed to control and larvae expressing 100× (A,C) or 36× (B,D) GR dipeptide repeats. More active zones and NMJ boutons are observed (A,C) and enhanced synaptic strength (EPSP amplitude; B,D) is observed following drug exposure. Unpaired t-test. **p<0.01, ***p<0.001.

FIG. 32A-D shows the results of testing the ability of Pikfyve ASO treatment to rescue neurodegeneration in TDP-43 mice. (A) Gait impairment, (B) Kyphosis, and (C) Tremor scoring in wild-type (WT) and TDP-43 homozygous mice treated with a negative control (NC) ASO or Pikfyve ASO at P0. Fore each assay, a score of 0 indicates no phenotype and 4 indicates the most severe phenotype. (D) qRT-PCR of whole brain of n=5 mice per group at P7 after ASO injection at P0. Unpaired t-test.

FIG. 33A-D provides qRT-PCR data showing that PIKFYVE ASOs suppress PIKFYVE mRNA levels in Hela cells in vitro. (A-D) PIKFYVE mRNA level with the ASO treatment specified relative to negative control ASO (NC ASO) treatment for PIKFYVE ASOs 19-50. PIKFYVE mRNA levels were normalized to HPRT expression as a housekeeping gene. The “HPRT ASO” sample is a positive control in which a validated ASO against the HPRT gene was used to confirm that transfection in the Hela cells resulted in gene suppression of HPRT if the HPRT-targeting ASO was used. 25 nM ASO was transfected using Lipofectamine 2000 and RNA was harvested 3 days later. RT-PCR was performed using Protoscript reverse transcriptase and iTaq SYBR green supermix (Bio-rad). “Lipofectamine only” is a negative control in which no ASO was used. “No transfection” is a negative control in which no transfection or ASO was used. Mean+/−s.d. of two biological replicates.

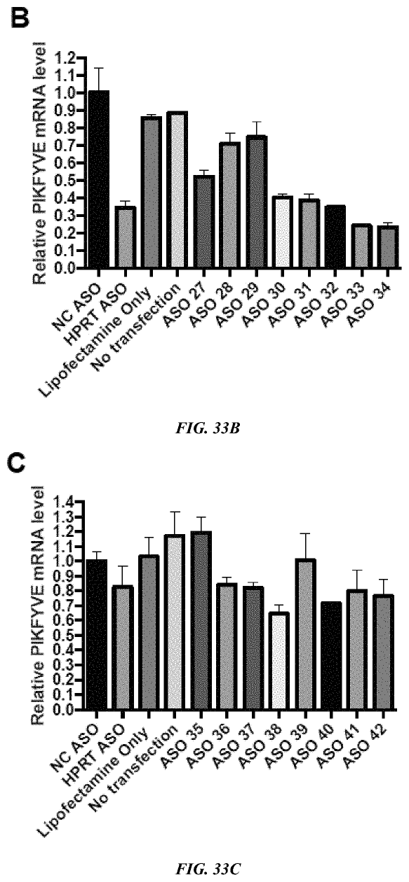

FIG. 34A-C presents qRT-PCR data showing that PIKFYVE ASOs suppress PIKFYVE mRNA levels in Hela cells in vitro. (A-C) PIKFYVE mRNA level with the ASO treatment specified relative to negative control ASO (NC ASO) treatment for PIKFYVE ASOs 51-74. PIKFYVE mRNA levels were normalized to HPRT expression as a housekeeping gene. The “HPRT ASO” sample is a positive control in which a validated ASO against the HPRT gene was used to confirm that transfection in the Hela cells resulted in gene suppression of HPRT if the HPRT-targeting ASO was used. 25 nM ASO was transfected using Lipofectamine 2000 and RNA was harvested 3 days later. RT-PCR was performed using Protoscript reverse transcriptase and iTaq SYBR green supermix (Bio-rad). “Lipofectamine only” is a negative control in which no ASO was used. “No transfection” is a negative control in which no transfection or ASO was used. Mean+/−s.d. of two biological replicates.

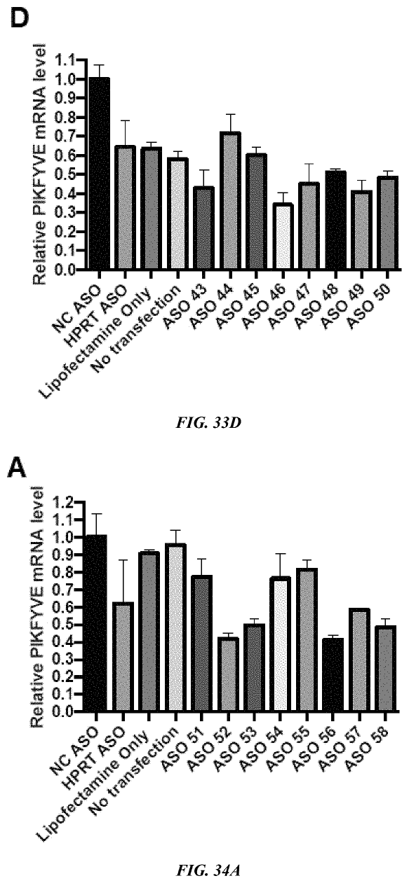

FIG. 35A-B provides qRT-PCR data showing that PIKFYVE ASOs suppress PIKFYVE mRNA levels in Hela cells in vitro. (A-B) PIKFYVE mRNA level with the ASO treatment specified relative to negative control ASO (NC ASO) treatment for PIKFYVE ASOs 75-109. PIKFYVE mRNA levels were normalized to HPRT expression as a housekeeping gene. The “HPRT ASO” sample is a positive control in which a validated ASO against the HPRT gene was used to confirm that transfection in the Hela cells resulted in gene suppression of HPRT if the HPRT-targeting ASO was used. 25 nM ASO was transfected using Lipofectamine 2000 and RNA was harvested 3 days later. RT-PCR was performed using Protoscript reverse transcriptase and iTaq SYBR green supermix (Bio-rad). “Lipofectamine only” is a negative control in which no ASO was used. “No transfection” is a negative control in which no transfection or ASO was used. Mean+/−s.d. of two biological replicates.

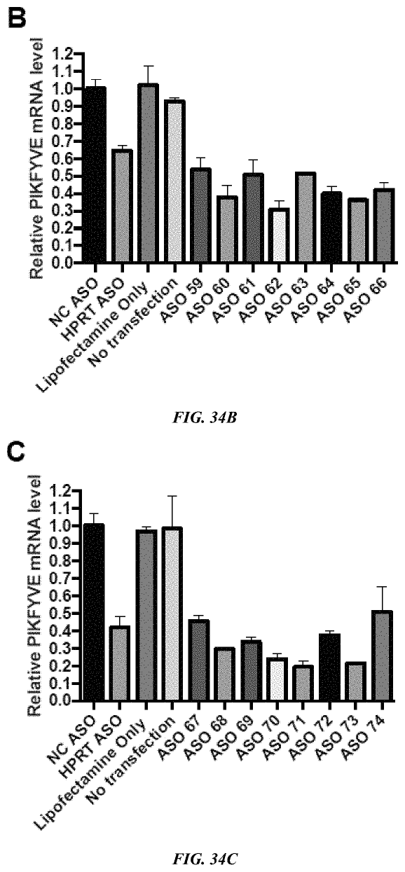

FIG. 36 presents qRT-PCR data showing that PIKFYVE ASOs suppress PIKFYVE mRNA levels in Hela cells in vitro. PIKFYVE mRNA level with the ASO treatment specified relative to negative control ASO (NC ASO) treatment for PIKFYVE ASOs 75-109. PIKFYVE mRNA levels were normalized to HPRT expression as a housekeeping gene. The “HPRT ASO” sample is a positive control in which a validated ASO against the HPRT gene was used to confirm that transfection in the Hela cells resulted in gene suppression of HPRT if the HPRT-targeting ASO was used. 25 nM ASO was transfected using Lipofectamine 2000 and RNA was harvested 3 days later. RT-PCR was performed using Protoscript reverse transcriptase and iTaq SYBR green supermix (Bio-rad). “Lipofectamine only” is a negative control in which no ASO was used. “No transfection” is a negative control in which no transfection or ASO was used. Mean+/−s.e.m. of three biological replicates. One-way ANOVA.

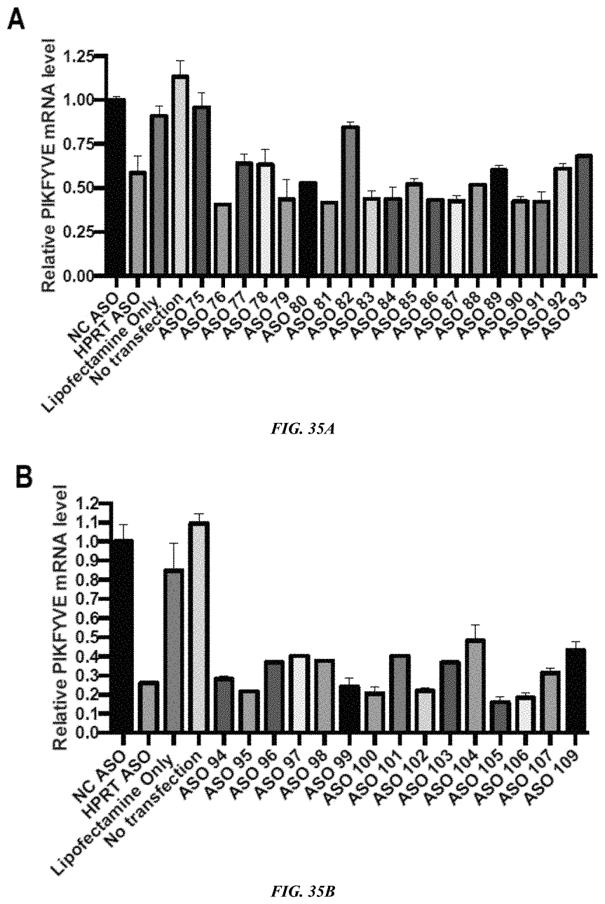

FIG. 37 shows qRT-PCR data showing that PIKFYVE ASOs suppress PIKFYVE mRNA levels in the brain in vivo. C57Bl/6 mice were generated harboring a bacterial artificial chromosome containing the full-length human PIKFYVE gene. 25 μg of negative control ASO (NC ASO) (N=7 mice, mixed gender) or hPIKFYVE ASO candidates (N=as indicated in the graph, mixed gender) were delivered to postnatal day 1 mice via intracerebroventricular injection. Tissues were collected on postnatal day 14 and processed for qRT-PCR analysis. The NC ASO group average served as the reference and Actin expression was used for normalization. PIKFYVE mRNA level with the ASO treatment specified relative to negative control ASO (NC ASO) treatment for each PIKFYVE ASO is show. RT-PCR was performed using Protoscript reverse transcriptase and iTaq SYBR green supermix (Bio-rad). Each data point represents one mouse and the number of mice per sample is indicated. Mean+/−s.e.m. of the number of mice shown. One-way ANOVA. Significance is shown for each PIKFYVE ASO compared to the negative control ASO samples. **p<0.01, ****p<0.0001.

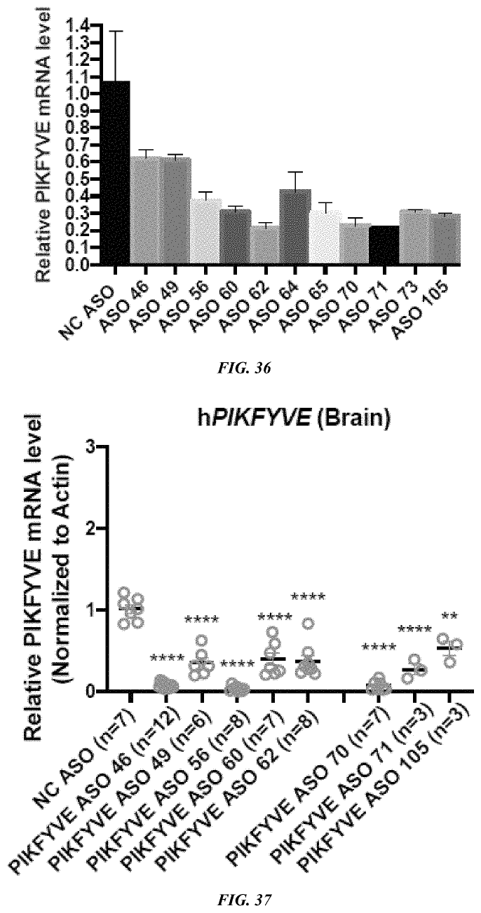

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The terms “comprise(s),” “include(s),” “having,” “has,” “can,” “contain(s),” “may” and variants thereof, as used herein, are intended to be open-ended transitional phrases, terms, or words that do not preclude the possibility of additional acts or structures.

The singular forms “a,” “an” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “antisense molecule” means an oligomeric nucleic acid or oligomeric duplex capable of achieving at least one antisense activity.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70%, at 80%, at least 90%, at least 95%, at least 98%, or at least 99% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, an MOE gapmer may comprise one or more modified internucleoside linkages and/or modified nucleobases and such modifications do not necessarily follow the gapmer pattern of the sugar modifications. Table 2, below, provides exemplary MOE-gapmers.

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif include the “5′ wing”, the “gap” and the “3′ wing” which form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise a number of nucleosides selected from 1, 2, 3, 4, 5, 6, 7, 8, 9, 10, or a range that includes or is between any two of the foregoing numbers (e.g., 1-5, 2-7, etc.). In certain embodiments, each nucleoside of each wing of a gapmer is a modified nucleoside.

In certain embodiments, the gap of a gapmer comprises comprise a number of nucleosides selected from 5, 6, 7, 8, 9, 10, 11, 12, 13, 14, 15, 16, 17, 18, 19, 20, 21, 22, 23, 24, 25, or a range that includes or is between any two of the foregoing numbers (e.g., 7-15, 10-20, etc.). In certain embodiments, each nucleoside of the gap of a gapmer is an unmodified 2′-deoxy nucleoside.

In certain embodiments, the gapmer is a deoxy gapmer. In further embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In another embodiments, modified oligonucleotides comprise, consist essentially of or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

“Inhibit” as used herein refers to the ability to substantially antagonize, prohibit, prevent, restrain, slow, disrupt, alter, eliminate, stop, or reverse the progression or severity of the activity of a particular agent (e.g., infectious agent) or disease.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages can be found in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003); Wan et al., Nuc. Acid. Res. 42, 13456 (2014); Chapter 10 of Locked Nucleic Acid Aptamers in Nucleic Acid and Peptide Aptamers: Methods and Protocols v 535, 2009 by Barciszewski et al., editor Gunter Mayerand; and WO 2017/015555. In certain embodiments, a population of modified oligonucleotides is enriched for modified oligonucleotides having at least one indicated phosphorothioate in the (Sp) configuration.

As used herein, “MOE” means methoxyethyl. “2′-MOE” means a —OCH₂CH₂OCH₃ group at the 2′ position of a furanosyl ring.

A “neurological disease” is any disease that causes electrical, biochemical, or structural abnormalities in the brain, spine, or neurons. For example, a neurological disease may be a neurodegenerative disease. The neurodegenerative disease may result in motor neuron degeneration, for example. The neurological disease may be amyloid lateral sclerosis, Huntington's disease, Alzheimer's disease, or frontotemporal dementia, for example. Further examples of neurological diseases include, but are not limited to Parkinson's disease, multiple sclerosis, peripheral myopathy, Rasmussen's encephalitis, attention deficit hyperactivity disorder, autism, central pain syndromes, anxiety, and/or depression, for example.

The neurological disease may be associated with aberrant endosomal trafficking. For example, endosomal pathways and endosomes are necessary components for the recycling or breakdown of membrane-bound proteins, trafficking of Golgi-associated proteins, and the extracellular release of proteins in exosomes. These processes aid neurotransmission and drive a balance between recycling and degradation of synaptic vesicles or neurotransmitter receptors, for example.

The neurological disease may be associated with aberrant lysosome degradation. Alterations in the lysosome degradation may be present in the neurological disease, such as a neurodegenerative disease. Cathepsin imbalance during aging and age-related diseases may provoke deleterious effects on CNS neurons and lysosomes may be sites for the unfolding and partial degradation of membrane proteins or their precursors that subsequently become expelled from a cell, or are released from dead cells and accumulate as pathological entities.

A health care professional may diagnose a subject as having a disease associated with motor neuron degeneration by the assessment of one or more symptoms of motor neuron degeneration. To diagnose a neurological disease, a physical exam may be followed by a thorough neurological exam. The neurological exam may assess motor and sensory skills, nerve function, hearing and speech, vision, coordination and balance, mental status, and changes in mood or behavior. Non-limiting symptoms of a disease associated with a neurological disease may be weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; change in appetite.

Tests may be performed to rule diseases and disorders that may have symptoms similar to those of neurological diseases, measure muscle involvement, assess neuron degeneration. Non limiting examples of tests are electromyography (EMG); nerve conduction velocity study; laboratory tests of blood, urine, or other substances; magnetic resonance imaging (MRI); magnetic resonance spectroscopy; muscle or nerve biopsy; transcranial magnetic stimulation; genetic screening; x-rays; fluoroscopy; angiography; computed tomography (CT); positron emission tomography; cerebrospinal fluid analysis; intrathecal contrast-enhanced CT scan; electroencephalography; electronystagmography; evoked response; polysomnogram; thermography; and ultrasound. A health care professional may also assess the patient's family history of diseases associated with motor neuron degeneration and make a diagnosis in part based on a familial history of neurological diseases. A healthcare professional may diagnose a disease associated with neurological disease in a subject after the presentation of one or more symptoms.

Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

Diseases associated with motor neuron degeneration may be a condition that results in the progressive destruction of motor neurons that interferes with neuronal signaling to the muscles, leading to muscle weakness and wasting. In healthy individuals, upper motor neurons transmit signals from the brain to lower motor neurons in the brain stem and spinal cord, which then transmit the signal to the muscles to result in voluntary muscle activity. The destruction of upper and lower motor neurons affects activity such as breathing, talking, swallowing, and walking, and overtime these functions can be lost. Examples of motor neuron diseases include, but are not limited to, amyotrophic lateral sclerosis, primary lateral sclerosis, progressive muscle atrophy, progressive bulbar palsy, and pseudobulbar palsy. The etiology of disease associated with motor neuron degeneration has not been fully elucidated and has been attributed to genetic factors and sporadic cases.

Neuronal hyperexcitability may occur when receptors for the excitatory neurotransmitter glutamate (glutamate receptors) such as the NMDA receptor and AMPA receptor are over-activated by excess glutamate or by other compounds or neurotransmitters acting on the glutamate receptors. Excitotoxicity may result from neuronal hyperexcitability. Excitotoxicity is the pathological process by which nerve cells are damaged or killed by excessive stimulation. The excessive stimulation allows high levels of calcium ions (Ca²⁺) to enter the cell. Ca²⁺ influx into cells activates a number of enzymes, including phospholipases, endonucleases, and proteases such as calpain. These enzymes can damage cell structures such as components of the cytoskeleton, membrane, and DNA.

Neuronal hyperexcitability may be involved in spinal cord injury, stroke, traumatic brain injury, hearing loss (through noise overexposure or ototoxicity), epilepsy, painful neuropathies, attention deficit hyperactivity disorder, autism, central pain syndromes, neurodegenerative diseases, multiple sclerosis, Alzheimer's disease, familial and sporadic amyotrophic lateral sclerosis (ALS), Parkinson's disease, familial and sporadic frontotemporal dementia, progressive supranuclear palsy, chronic traumatic encephalopathy, Charcot Marie Tooth 2A and 4B, schizophrenia, Rasmussen's encephalitis, Huntington's disease, alcoholism or alcohol withdrawal and especially over-rapid benzodiazepine withdrawal, and also Huntington's disease. Other common conditions that cause excessive glutamate concentrations around neurons are hypoglycemia. Blood sugars are the primary glutamate removal method from inter-synaptic spaces at the NMDA and AMPA receptor site.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” or “mC” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 2,6-diaminopurine, 5-hydroxymethylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides have 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range that includes or is between of any two of the foregoing numbers, linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“PIKFYVE”, also known in the art as “phosphatidylinositol-3-phosphate 5-kinase type III” or “PIPKIII”, is a FYVE finger-containing phosphoinositide kinase encoded by the PIKFYVE gene. PIKFYVE is a highly evolutionarily conserved lipid kinase and also has protein kinase activity, which regulates endomembrane homeostasis and plays a role in the biogenesis of endosome carrier vesicles from early endosomes. PIKFYVE-mediated conversion of PI3P to PI(3,5)P₂ blocks recruitment of the protein EEA1. The recruitment is blocked, because PIP3 is needed to form a platform with RAB5 that enables anchoring of EEA1 to early endosomes. EEA1 then drives fusion with endocytic and other endosomal vesicles. Mutations in PIKFYVE are known to lead to Francois-Neetens corneal fleck dystrophy. Disruption of both Pikfyve alleles is embryonic lethal in mice at the pre-implantation state of the embryo. A link between type 2 diabetes and PIKFYVE is inferred by the observations that PIKFYVE perturbation inhibits insulin-regulated glucose uptake.

As used herein a “PIKFYVE disease or disorder” includes variously lysosomal degradation diseases and disorders. For example, the a PIKFYVE disease or disorder includes, but is not limited to, amyloid diseases (such as Alzheimer's disease, Parkinson's disease, Huntington's disease, type 2 diabetes, diabetic amyloidosis and chronic hemodialysis-related amyloid), multiple sclerosis, and an MPS disorder (such as MPS I, MPS II, MPS IIIA, MPS IIIB, MPS IIIC, MPS HID, MPS IVA, MPS IVB, MPS VI, MPS VII, or MPS IX). In some embodiments, the diseases are autoimmune disorders (such as multiple sclerosis, rheumatoid arthritis, juvenile chronic arthritis, Ankylosing spondylitis, psoriasis, psoriatic arthritis, adult still disease, Bechet syndrome, familial Mediterranean fever, Crohn's disease, leprosy, osteomyelitis, tuberculosis, chronic bronchiectasis, Castleman disease), or CNS disorders (such as spongiform encephalopathies (Creutzfeldt-Jakob, Kuru, Mad Cow)). In some embodiments, the disease or disorder is selected from the group consisting of familial and/or sporadic amyotrophic lateral sclerosis (ALS), familial and/or sporadic frontotemporal dementia, Progressive supranuclear palsy, Alzheimer's disease, Chronic traumatic encephalopathy, Parkinson's disease, Charcot Marie Tooth 2A and 4B, and Huntington's disease. The compositions, methods, and kits of the disclosure can be used to treat individuals with lysosomal storage diseases comprising administering to a subject in need of treatment a therapeutically effective amount of an antisense or inhibitory nucleic acid that inhibits the activity of PIKFYVE. In some embodiments, the compositions of the disclosure decrease or inhibit the activity of PIKFYVE and alters the biogenesis, function or dynamics of the endosomal or lysosomal systems in a way that reduces the abundance of the material abnormally stored in the lysosome in lysosomal storage diseases. In some embodiments, the antisense or inhibitory nucleic acids target, decrease or inhibit the activity of PIKFYVE thus altering the biogenesis, functions, or dynamics of the endoplasmic reticulum or Golgi apparatus in a way that reduces the abundance of the material abnormally stored in the lysosome in lysosomal storage diseases. In other embodiments, the disease is a neurological disorder.

As used herein, “RNAi compound” which includes “inhibitory nucleic acids” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, —OCN, —CF₃, —OCF₃, —O—C₁₋₁₀ alkoxy, —O—C₁₋₁₀ substituted alkoxy, —O—C₁₋₁₀ alkyl, —O—C₁₋₁₀ substituted alkyl, —S-alkyl, —N(Rₘ)-alkyl, —O-alkenyl, —S-alkenyl, —N(Rₘ)-alkenyl, —O-alkynyl, —S-alkynyl, —N(Rₘ)-alkynyl, —O-alkylenyl-O-alkyl, alkynyl, alkaryl, aralkyl, —O-alkaryl, —O-aralkyl, —O(CH₂)₂SCH₃, —O(CH₂)₂ON(Rₘ)(Rₙ) or —OCH₂C(═O)—N(Rₘ)(Rₙ), where each Rₘ and Rₙ is, independently, H, an amino protecting group, or substituted or unsubstituted C₁₋₁₀ alkyl, and the 2′-substituent groups can be further substituted with one or more substituent groups independently selected from among: hydroxyl, amino, alkoxy, carboxy, benzyl, phenyl, nitro, thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), and alkyl. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the like.

In certain embodiments, a 2′-substituted non-bicyclic modified nucleoside comprises a sugar moiety comprising a non-bridging 2′-substituent group selected from: F, NH₂, N₃, —OCF₃, —OCH₃, —O(CH₂)₃NH₂, —CH₂CH═CH₂, —OCH₂CH═CH₂, —OCH₂CH₂OCH₃, —O(CH₂)₂SCH₃, —O(CH₂)₂ON(Rₘ)(Rₙ), —O(CH₂)₂O(CH₂)₂N(CH₃)₂, and N-substituted acetamide (—OCH₂C(═O)—N(Rₘ)(Rₙ)), where each Rₘ and Rₙ is, independently, H, an amino protecting group, or substituted or unsubstituted C₁₋₁₀ alkyl.

In certain embodiments, a 2′-substituted nucleoside non-bicyclic modified nucleoside comprises a sugar moiety comprising a non-bridging 2′-substituent group selected from: F, —OCF₃, —OCH₃, —OCH₂CH₂OCH₃, —O(CH₂)₂SCH₃, —O(CH₂)₂ON(CH₃)₂, —O(CH₂)₂O(CH₂)₂N(CH₃)₂, and —OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, —OCH₃, and —OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′- (CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof, 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b}, is, independently, H, a protecting group, or C₁₋₁₂ alkyl.

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁₋₁₂ alkyl, substituted C₁₋₁₂ alkyl, C₁₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, heterocycle radical, substituted heterocycle radical, heteroaryl, substituted heteroaryl, C₅₋₇ alicyclic radical, substituted C₅₋₇ alicyclic radical, halogen, OJ₁, NJ₁₋₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁₋₁₂ alkyl, substituted C₁₋₁₂ alkyl, C₂₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, acyl (C(═O)—H), substituted acyl, a heterocycle radical, a substituted heterocycle radical, C₁₋₁₂ aminoalkyl, substituted C₁₋₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. Pat. No. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patent Publication Nos. Allerson et al., US2008/0039618 and Migawa et al., US2015/0191727.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human subject. The subject or patient may be undergoing other forms of treatment.

As used herein, “target nucleic acid” and “target RNA” mean a nucleic acid that an antisense compound is designed to affect.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, mammal, or human, such as reducing and/or inhibiting the function of a receptor. A therapeutically effective amount may be administered in one or more administrations (e.g., the agent may be given as a preventative treatment or therapeutically at any stage of disease progression, before or after symptoms, and the like), applications or dosages and is not intended to be limited to a particular formulation, combination or administration route. It is within the scope of the present disclosure that the drug may be administered at various times during the course of treatment of the subject. The times of administration and dosages used will depend on several factors, such as the goal of treatment (e.g., treating v. preventing), condition of the subject, etc. and can be readily determined by one skilled in the art.

As used herein, the term “treat” or “treating” a subject, refers to administering a composition or agent described herein to the subject, such that at least one symptom of a disease or disorder is healed, alleviated, relieved, altered, remedied, reduced, ameliorated, or improved. Treating includes administering an amount effective to alleviate, relieve, alter, remedy, reduce, ameliorate, and/or improve one or more symptoms associated with a disease or disorder. The treatment may inhibit deterioration or worsening of a symptom associated with the disease or disorder.

“Haploinsufficiency” or “haploinsufficient” as used herein may refer to when a diploid organism has only a single functional copy of a gene (with the other copy inactivated or suppressed by mutation (e.g., expansion, deletion, substitution, etc.)) and the single functional copy does not produce enough of a gene product (typically a protein) to bring about a wild-type condition, leading to an abnormal or diseased state.

PIKFYVE is a lipid kinase that regulates vesicle trafficking, including autophagosome-lysosome. Surprisingly, the results presented herein suggest that the therapeutic mechanism of PIKFYVE inhibition is to prevent autophagosome-lysosome fusion. Blocking autophagosome-lysosome fusion induces secretory autophagy, or exosomal release, in motor neurons, which robustly clears misfolded proteins including C9ORF72 dipeptide repeat proteins (DPRs) and TDP-43 through exosomes. The accumulation of misfolded proteins can induce neuron death and is a common feature of neurodegenerative diseases. Through unbiased phenotypic screens on patient-derived neurons, it was found herein that one of the most significant ways to rescue ALS iMN survival is by PIKFYVE inhibition and secretory autophagy. The data presented herein establishes that PIKFYVE suppression and secretory autophagy are viable and important therapeutic targets for treating neurodegenerative diseases. Examples of such neurodegenerative diseases, include but are not limited to,

Frontotemporal dementia (FTD) is a complex disease that results from many diverse genetic etiologies. There are no drugs that slow the progression of FTD. Although emerging therapeutic strategies that specifically target causal gene mutations may protect against individual forms of FTD, these approaches cannot address the vast majority of cases that have unknown genetic etiology. Moreover, given the large number of different genes that cause FTD and the fact that each form is rare, this strategy may be difficult to implement for all cases. Thus, new therapeutic strategies that rescue multiple forms of FTD, including those with unknown genetic etiologies, are needed.

ALS, FTD, and Alzheimer's are complex diseases that each result from many diverse genetic etiologies. Although therapeutic strategies that target specific causal mutations (e.g., SOD1 ASOs) may prove effective against individual forms of ALS and FTD, these approaches cannot address the vast majority of cases that have unknown genetic etiology. Moreover, given the large number of different genes that likely contribute to ALS and FTD and the fact that each genetic form is relatively rare this strategy may be difficult to implement for all cases. Thus, there is a pressing need for new therapeutic strategies for treating subjects with neurogenerative diseases, including treatments for the multiple forms of ALS and FTD, particularly those with unknown genetic etiologies.

The accumulation of misfolded proteins can induce neuron death and is a common feature of neurodegenerative diseases (e.g., ALS, FTD, Alzheimer's disease, etc.). To mitigate misfolded protein accumulation, studies have sought to stimulate known proteostasis pathways including the ubiquitin-proteosome system and autophagy. However, these pathways decline during aging and may be difficult to rescue effectively.

To identify new therapeutic targets that rescue multiple forms of ALS, unbiased chemical screens were performed to search for targets that can rescue the degeneration of iMNs from multiple C9ORF72 and sporadic ALS patients. It was found herein that inhibitors of PIKFYVE kinase were among the most potent and broadly-efficacious compounds across patient lines. Surprisingly, the therapeutic mechanism of PIKFYVE inhibition is in part due to blocking autophagosome-lysosome fusion, which induces secretory autophagy and robustly clears misfolded proteins including C9ORF72 DPRs and TDP-43 through exosomal secretion, the third proteostasis pathway. That secretory autophagy is one of the most potent ways to prevent neurodegeneration in ALS, FTD, and Alzheimer's disease differs from mainstream thinking in the field.

In studies presented herein, it was confirmed that secretory autophagy is a therapeutic mechanism of PIKFYVE inhibition. It was further found herein that PIKFYVE suppression with antisense oligonucleotides had efficacy in ALS, FTD, and Alzheimer's disease patient-derived neurons by inducing secretory autophagy. Thus, inducing secretory autophagy is a highly effective, and new therapeutic strategy for treatment of neurodegenerative diseases, including the diverse forms of ALS, FTD, and Alzheimer's disease. It is expected that the foregoing therapeutic strategies may also be effective for other non-neurodegenerative diseases driven by aberrant protein accumulation, including, but not limited to, familial and sporadic amyotrophic lateral sclerosis (ALS), familial and sporadic frontotemporal dementia (FTD), progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubro-pallidoluysian atrophy, spinocerebellar ataxias, and Creutzfeldt-Jakob disease.

Disclosed herein are methods of treatment may comprise administering to a subject in need thereof a composition comprising an effective amount of one or more antisense oligonucleotides or inhibitor oligonucleotides that treats neurological diseases by inhibiting PIKFYVE expression. The one or more antisense oligonucleotides or inhibitor oligonucleotides may decrease or inhibit neurodegeneration. The one or more antisense oligonucleotides or inhibitor oligonucleotides may decrease neuronal hyperexcitability.

The composition may inhibit kinase activity by inhibiting expression of a kinase. The composition may inhibit PIKFYVE kinase activity or expression. The one or more antisense oligonucleotides or inhibitor oligonucleotides can be combined with small molecule therapeutics. The PIKFYVE kinase small molecule inhibitor may be apilimod. The PIKFYVE kinase small molecule inhibitor may be YM201636. The PIKFYVE kinase small molecule inhibitor may be a combination of apilimod and YM201636. The disclosure provides oligonucleotides (modified or unmodified) that can be used to modulate PIKFYVE expression (see Table 1).

TABLE 1
(5′ to 3′) generic sequences useful
in designing PIKFYVE kinase
antisense or inhibitory nucleic
acids.

| Sequence | |
|---|---|
| ID number | Sequence |
| 1 | ACTTGTCCATTGCCTCACAA |
| 2 | TCTCGGTCCCCTCCTTGCCC |
| 3 | CCTTCACTTGGCTGTCTGTG |
| 4 | CTCATGCCCTCGTCCCTCAA |
| 5 | TTTCCTTCACTTGGCTGTCT |
| 6 | TTGTCTGTCCTCCACTCATT |
| 7 | TTTCATGCCACTTCCCAACC |
| 8 | TGCCCATTTCGGATTAGCCA |
| 9 | GCTGGTCCAACTTCCACTCA |
| 10 | CATCCTCAGTGGTCTCTGTC |
| 11 | CATGCCCTCGTCCCTCAATC |
| 12 | ATCCTCAGTGGTCTCTGTCC |
| 13 | CTTCCCCTTGTCTCAGTCTT |
| 14 | TTCGTAAGCAGCTGGTCCAT |
| 15 | ATGACTTCAGGTGGCGTAGG |
| 16 | CTTGATTCTCGGTCCCCTCC |
| 17 | GAGCCTTGCTTGCATCTTCT |
| 18 | TGGTCCTTTGTTCCTGTCCC |
| 19 | TTGGTCCTTTGTTCCTGTCC |
| 20 | CTCCAACTTTAGCCCTTTGA |
| 21 | TGCCTCAGCCACCCAAGTAG |
| 22 | GTGTAACTGAACTTGTCCAG |
| 23 | GTCATAAGTCCTTGGTCAAC |
| 24 | CTTCCAGTTCAAAACAAACC |
| 25 | CCTAAACAAGAGCAGCAAGG |
| 26 | ACTCACTGCTAAATCATGGA |
| 27 | ACAGTCCACCTTTGTTGACC |
| 28 | AAATGCCCATCAAACACATC |
| 29 | TCTACTTCATCTTGAGGCTC |
| 30 | CTGTTGTCCTCCAGTGTCAG |
| 31 | GATGCCAGCCCAAAGGTGTG |
| 32 | TAGCTGAAAGCAACCTCTCC |
| 33 | GCAACTGCTGGAGTAGTGCC |
| 34 | AGTCACTATGGAGCAACTGC |
| 35 | ATGATGTCCCTCCAAGATGA |
| 36 | CTTTCCTGAAGCACAATAGG |
| 37 | CAATCCAAGGACTGACACAG |
| 38 | AACTGGTGTTACCTGTTTGC |
| 39 | TTCTTCCATCTCTTTCTGTG |
| 40 | GCAACCTGTTATTCCAAGCT |
| 41 | CTGGATCAAAAGGAAATGGA |
| 42 | TTACAACTGAGAGCAAAAGC |
| 43 | GTACTATTTGTTGGAAGCCC |
| 44 | ATTGAAGTTCCACATGAGGA |
| 45 | AGCCACAGAACTGAAAAGCC |
| 46 | TCATGCCACTTCCCAACCTA |
| 47 | GCAGCTCTACAAATGCTTTC |
| 48 | CAGAACTAGCCATTATGTAG |
| 49 | GGTTTAGTAACTACAGACTC |
| 50 | CACTCAATTTCTTGGATTGG |
| 51 | GGTCTTGAACTCCTGACCTC |
| 52 | GCTATTATTATAGGCAGTCC |
| 53 | CCTCAGTGTTTTGTGTATCT |
| 54 | TTATTGTCAGAAGCCATTCC |
| 55 | CACAAAGTTACCTACTAGAC |
| 56 | CTGACATAGTTTTCTCTTGC |
| 57 | CATGAAGCTGATGACCACAA |
| 58 | TTTCACTCTTCAAGCTCAGC |
| 59 | CTTGAGGAATAATGAATGGC |
| 60 | ATACACTGCACCTGTGTTAC |
| 61 | CAAAACTGACACAGCAGTAT |
| 62 | GCTAGAACCAAAGTAGAGAA |
| 63 | GAGATGAGTAGAACTGGTGG |
| 64 | CACTGCTGTCCAGAATCACT |
| 65 | GGACTGTCTGAACAACCTGG |
| 66 | GGACTGGAAACTGCTGACTG |
| 67 | GGCTGCTACTCTTTGGTCTG |
| 68 | CCTGGTAGTAAGCACTATCT |
| 69 | AACAGGCAAGCTGGAAAGAT |
| 70 | GCCAGGATAGTCAATTTATG |
| 71 | CCTTGGCAGTTTTACACAGG |
| 72 | TCTCCAAAAGTTCAAGGCAG |
| 73 | GGTTACAGGTATATGCCACC |
| 74 | CCAGGTTCAAACAATTCTCC |
| 75 | TCTTGCTCTGTTGCCCAGGC |
| 76 | ATGGATGGAGTGACCACAGG |
| 77 | AATGACATAGACCAGGACTC |
| 78 | GGAACTTAGCATCACATTAG |
| 79 | GGAGTGGAACACATACTTCA |
| 80 | GCAAGTCTTTCATCAATGGC |
| 81 | CAGAGACTTTGTTTCTTGGC |
| 82 | TTCTCCATTCTGAAGTCCTG |
| 83 | CTCTGGCTGGACAAAGTTGT |
| 84 | GGACAACATTCCAGAAGCCT |
| 85 | CAGTAAAACTTGGCATTAGC |
| 86 | CAGTGTTGTTCTGAGAGTTC |
| 87 | AAGACATGAGCCTTGCTTGC |
| 88 | TGGAACACTTCAACAGAAGA |
| 89 | TAGGCAACTGAAATATCTGC |
| 90 | ACATCACTAAATCACCTTGG |
| 91 | ATTTGTGACAAGTGCCCAGG |
| 92 | GGTGCTGTGGACAACCTTCA |
| 93 | TCATAATCAGAGCCTCCTCT |
| 94 | GAGAATGATAAGCAACACAG |
| 95 | AGGCATAGCAAATTCATCCA |
| 96 | TTCCTGCTCACCCTTCTCAA |
| 97 | CAAACTGCTATCATCACAGG |
| 98 | TGCTTCACAGAGGCAACAGC |
| 99 | TCTTGTTGGTCATCCACAGG |
| 100 | CAGGACTTGGAGGAACTGAA |
| 101 | CTGGCTGTATCTAGCTCAGG |
| 102 | CAAAGATGGCTTTCATGGTT |
| 103 | TATAATCCAGTGTCATCCTG |
| 104 | GAGATACAGAGGATCACATC |
| 105 | AGACCAGTAAACCTGCTCTG |
| 106 | CATGCCCTGAAGTCCAGAGA |
| 107 | CAGAAGAGCTGCTGAAGAGC |
| 108 | GCCTGAAACAGTATCTCTCT |
| 109 | GCAGCTCTTTGGATTTTTCC |
| 110 | ACCTGGCAGACCAATGACAC |
| ill | CATCATCCTGGTTCTTGACA |
| 112 | CAAACAAAGCCATTGACAAC |
| 113 | TCTACCCAAGCTCTGGCTAT |
| 114 | CAGAACATGCTTGGACACTG |
| 115 | ATCCAACTCCTTCAGGATTA |
| 116 | GCAATGAGTGACTCAAAGAC |
| 117 | CAGACATGACTTCAGGTGGC |
| 118 | TTTCCCTGCTGTCCCTCTGA |
| 119 | GAAGGCAGCTCCTGATTTGC |
| 120 | GGAGATTTTCATCTAGCAGG |
| 121 | CTATAATGAGGTGGCTAGAA |
| 122 | CCAACTACTAGCTCATTGCT |
| 123 | CTCACAAAACCTAGTCCTGT |
| 124 | TTTCAGCAATTCAGACCCAA |
| 125 | GACAGCTCCACAGGTATCAA |
| 126 | AACAAATTCAGCAGCTCTAC |
| 127 | ATAGGCAGTCCAAAAACAGT |
| 128 | TTTGCCACAAAGTTACCTAC |
| 129 | GCACCTGTGTTACCAAAAAA |
| 130 | AAGTCTCCAAAAGTTCAAGG |
| 131 | GTCACTTATTGAGAGCTGTT |
| 132 | TTTGATGTGGACAAACTGAC |
| 133 | GTAGAGATACTCAATGGAAC |
| 134 | TCCAGCAAACTGCTATCATC |
| 135 | TCACCCTTCTCAAACACAAT |
| 136 | TGGCCTCCTTCTGCTCTCTC |

In one embodiment, the disclosure provides modified oligonucleotides comprising 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range that includes or is between of any two of the foregoing numbers, linked nucleosides, and having a nucleobase sequence comprising at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-136 in Table 1. In some embodiments, the modified oligonucleotide is at least 80% to 100% (i.e., 80%, 82%, 84%, 86%, 88%, 90%, 92%, 94%, 96%, 98% or 100%; or any numerical range or value between any of the foregoing values) identical to any of the sequences comprising or consisting of SEQ ID NO:1-136.

The sequences provided in Table 1 can be used to design antisense molecules for inhibition of PIKFYVE expression. For example, gapmer oligonucleotides can be designed using the sequences in Table 1 and can comprise a 5′-wing of about 3-5 nucleotides, a 3′-wing of about 3-5 nucleotides and a gap region comprising 8-12 consecutive deoxyribonucleosides of any one of the sequences of Table 1. In one embodiment, an oligonucleotide of the disclosure comprises a gapmer having a gap segment of at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-136 in Table 1; flanked by a 5′ and 3′ wing segments, wherein the gap segment is located between the 5′ and 3′ wing segments and wherein each of the wing segments comprises a modified sugar. In one embodiment, the gap segment is 8-10 nucleosides in length and each wing segment is 3-5 modified nucleosides in length. In yet another embodiment, an oligonucleotide of the disclosure comprises a 5′ wing segment comprising modified sugars and having the nucleobase sequence of the first 3-5 nucleobases of any of SEQ ID NO:1-136, followed by a gap of the next 8-12 unmodified nucleotides of the same sequence corresponding to SEQ ID NO:1-136, followed by a 3′ wing segment comprising modified sugars and having the nucleobase sequence of the last 3-5 nucleobases of the same sequence corresponding to SEQ ID NO:1-136. Table 2 provides MOE gapmers of the disclosure.

The 5′ and/or 3′ wings can comprise the following chemistries: 2′-OMe, 2′-MOE, LNA or DNA, by themselves or used in combination with one another. The backbone linkage of the 5′ and/or 3′ wings can be phosphorothioate or a mixture of phosphodiester and phosphorothioate. Any combination of phosphorothioate and phosphodiester linkages can be found throughout the wing and gap regions (i.e., any position in the oligo can have either a phosphorothioate or phosphodiester linkage).

In some embodiments, the oligonucleotide is single stranded. In some embodiments the oligonucleotide comprises or is complexed with a moiety that neutralizes charge on the oligonucleotide to promote uptake and transfer across a cell membrane.

TABLE 2
PIKFYVE Antisense Oligonucleotide Sequences (ASOs). (Gapmer design: 5′-
five 2′-methoxy ethylribose nucleotides-ten DNA nucleotides-five 2′-methoxyethylribose
nucleotides-3′; /i2MOErN/ = 2′-methoxyethylribose nucleotide; A, C, T, G = adenine,
cytosine, thymine, guanosine, respectively; * = phosphorothioate linkages)(Note that the
following table provide 2′MOE wings, however, alternative wings comprising 2-OMe, LNA
etc. are contemplated)

PIKFYVE ASO1
/52MOErA/*/i2MOErC/*/i2MOErT/*/i2MOErT/*/i2MOErG/T*C*C*A*T*T*G*C*C*T
*/i2MOErC/*/i2MOErA/*/i2MOErC/*/i2MOErA/*/32MOErA/
PIKFYVE ASO2
/52MOErT/*/i2MOErC/*/i2MOErT/*/i2MOErC/*/i2MOErG/*G*T*C*C*C*C*T*C*C*T
*/i2MOErT/*/i2MOErG/*/i2MOErC/*/i2MOErC/*/32MOErC/
PIKFYVE ASO3
/52MOErC/*/i2MOErC/*/i2MOErT/*/i2MOErT/*/i2MOErC/A*C*T*P*G*G*C*T*G*T
*/i2MOErC/*/i2MOErT/*/i2MOErG/*/i2MOErT/*/32MOErG/
PIKFYVE ASO4
/52MOErC/*/i2MOErT/*/i2MOErC/*/i2MOErA/*/i2MOErT/G*C*C*C*T*C*G*T*C*C
*/i2MOErC/*/i2MOErT/*/i2MOErC/*/i2MOErA/*/32MOErA/
PIKFYVE ASO5
/52MOErT/*/i2MOErT/*/i2MOE*T/*/i2MOErC/i2MOEC/T*T*C*A*C*T*T*G*G*C
*/i2MOErT/*/i2MOErG/*/i2MOErT/*/i2MOErC/*/32MOErT/
PIKFYVE ASO6
/52MOErT/*/i2MOErT/*/i2MOErG/*/i2MOErT/*/i2MOErC/*T*G*T*C*C*T*C*C*A*C
*/i2MOErT/*/i2MOErC/*/i2MOErA/*/i2MOErT/*/32MOErT/
PIKFYVE ASO7
/52MOErT/*/i2MOErT/*/i2MOErT/*/i2MOE/C/A2MOEA/T*G*C*C*A*C*T*T*C*C
*/i2MOErC/*/i2MOErA/*/i2MOErA/*/i2MOErC/*/32MOErC/
PIKFYVE ASO8
/52MOErT/*/i2MOErG/*/i2MOErC/*/i2MOErC/*/i2MOEC/A*T*T*T*C*G*G*A*T*T
*/i2MOErA/*/i2MOErG/*/i2MOErC/*/i2MOErC/*/32MOErA/
PIKFYVE ASO9
/52MOErG/*/i2MOErC/*/i2MOENT/*/i2MOErG/*/i2MOErG/T*C*C*A*A*C*T*T*C*
C*/i2MOErA/*/i2MOErC/*/i2MOErT/*/i2MOErC/*/32MOErA/
PIKFYVE ASO10
/52MOErC/*/i2MOErA/*/i2MOErT/*/i2MOErC/*/i2MOEC/*T*C*A*G*T*O*G*T*C*T
*/i2MOErC/*/i2MOETT/*/i2MOErG/*/i2MOErT/*/32MOErC/
PIKFYVE ASO11
/52MOErC/*/i2MOErA/*/i2MOErT/*/i2MOErG/*/i2MOErC/*C*C*T*C*G*T*C*C*C*T
*/i2MOErC/*/i2MOErA/*/i2MOErA/*/i2MOErT/*/32MOErC/
PIKFYVE ASO12
/52MOErA/*/i2MOErT/*/i2MOErC/*/i2MOErC/*/i2MOErT/*C*A*G*T*G*G*T*C*T*C
*/i2MOErT/*A2MOErG/*A2MOErT/*/i2MOErC/*/32MOErC/
PIKFYVE ASO13
/52MOErC/*/i2MOErT/*/i2MOErT/*/i2MOErC/*/i2MOErC/C*C*T*T*G*T*C*T*C*A
*/i2MOErG/*/i2MOErT/*A2MOErC/*/i2MOErT/*/32MOErT/
PIKFYVE ASO14
/52MOErT/*/i2MOErT/*/i2MOErC/*/i2MOErG/*/i2MOErT/*A*A*G*C*A*G*C*T*G*
G*/i2MOErT/*/i2MOErC/*/i2MOErC/*/i2MOErA/*/32MOErT/
PIKFYVE ASO15
/52MOErA/*/i2MOErT/*/i2MOErG/*/i2MOErA/*/i2MOEC/T*T*C*A*G*G*T*G*G*
C*A2MOErG/*A2MOErT/*/i2MOErA/*/i2MOErG/*/32MOErG/
PIKFYVE ASO16
/52MOErC/*/i2MOErT/*/i2MOErT/*/i2MOErG/N2MOErA/T*T*C*T*C*G*G*T*C*C
*/i2MOErC/*/i2MOErC/*/i2MOErT/*/i2MOErC/*/32MOErC/
PIKFYVE ASO17
/52MOErG/*/i2MOErA/*/i2MOErG/*/i2MOErC/*/i2MOEC/*T*T*G*C*T*T*G*C*A*
T*/i2MOErC/*/i2MOErT/*/i2MOErT/*/i2MOErC/*/32MOErT/
PIKFYVE ASO18
/52MOErT/*/i2MOErG/*/i2MOErG/*/i2MOErT/*/i2MOErC/C″P*TTG*T*T*C*C*T
*/2MOErG/*/2MOErT/*/i2MOErC/*/2MOErC/*/32MOErC/
PIKFYVE ASO23
TTGGTCCTTTGTTCCTGTCC
/52MOErT/*/i2MOErT/*/i2MOErG/*/i2MOErG/*/i2MOErT/*C*C*T*T*T*G*T*T*C*C
*/i2MOErT/*/i2MOErG/*/i2MOErT/*/i2MOErC/*/32MOErC/
PIKFYVE ASO26
CTCCAACTTTAGCCCTTTGA
/52MOErC/*/i2MOErT/*/i2MOErC/*/i2MOErC/*/i2MOErA/*A*C*T*T*T*A*G*C*C*C
*/i2MOErT/*/i2MOErT/*/i2MOErT/*/i2MOErG/*/32MOErA/
PIKFYVE ASO27
TGCCTCAGCCACCCAAGTAG
/52MOErT/*/i2MOErG/*/i2MOErC/*/i2MOErC/*/i2MOErT/*C*A*G*C*C*A*C*C*C*
A*/i2MOErA/*/i2MOErG/*/i2MOErT/*/i2MOErA/*/32MOErG/
PIKFYVE ASO29
GTGTAACTGAACTTGTCCAG
/52MOErG/*/i2MOErT/*/i2MOErG/*/i2MOErT/*/i2MOEA/*A*C*T*G*A*A*C*T*T*
G*/i2MOErT/*/i2MOErC/*/i2MOErC/*/i2MOErA/*/32MOErG/
PIKFYVE ASO32
GTCATAAGTCCTTGGTCAAC
/52MOErG/*/i2MOErT/*/i2MOErC/*/i2MOErA/*/i2MOErT/*A*A*G*T*C*C*T*T*G*
G*/i2MOErT/*/i2MOErC/*/i2MOErA/*/i2MOErA/*/i2MOErC/
PIKFYVE ASO33
CTTCCAGTTCAAAACAAACC
/52MOErC/*/i2MOErT/*/i2MOErT/*/i2MOErC/*/i2MOErC/*A*G*T*T*C*A*A*A*A*
C*/i2MOErA/*/i2MOErA/*/i2MOErA/*/i2MOErC/*/32MOErC/
PIKFYVE ASO37
CCTAAACAAGAGCAGCAAGG
/52MOErC/*/i2MOErC/*/i2MOErT/*/i2MOErA/*/i2MOErA/*A*C*A*A*G*A*G*C*A*
G*/i2MOErC/*/i2MOErA/*/i2MOErA/*/i2MOErG/*/32MOErG/
PIKFYVE ASO38
ACTCACTGCTAAATCATGGA
/52MOErA/*/i2MOErC/*/i2MOErT/*/i2MOErC/*/i2MOErA/*C*T*G*C*T*A*A*A*T*
C*/i2MOErA/*/i2MOErT/*/i2MOErG/*/i2MOErG/*/32MOErA/
PIKFYVE ASO45
ACAGTCCACCTTTGTTGACC
/52MOErA/*/i2MOErC/*/i2MOErA/*/i2MOErG/*/i2MOErT/*C*C*A*C*C*T*T*T*G*T
*/i2MOErT/*/i2MOErG/*/i2MOErA/*/i2MOErC/*/32MOErC/
PIKFYVE ASO48
AAATGCCCATCAAACACATC
/52MOErA/*/i2MOErA/*/i2MOErA/*/i2MOErT/*/i2MOEG/*C*C*C*A*T*C*A*A*A*
C*/i2MOErA/*/i2MOErC/*/i2MOErA/*/i2MOErT/*/32MOErC/
PIKFYVE ASO50
TCTACTTCATCTTGAGGCTC
/52MOErT/*/i2MOErC/*/i2MOErT/*/i2MOErA/*/i2MOErC/*T*T*C*A*T*C*T*T*G*A
*/i2MOErG/*/i2MOErG/*/i2MOErC/*/i2MOErT/*/32MOErC/
PIKFYVE ASO53
CTGTTGTCCTCCAGTGTCAG
/52MOErC/*/i2MOErT/*/i2MOErG/*/i2MOErT/*/i2MOErT/*G*T*C*C*T*C*C*A*G*T
*/i2MOErG/*/i2MOErT/*/i2MOErC/*/i2MOErA/*/32MOErG/
PIKFYVE ASO56
GATGCCAGCCCAAAGGTGTG
/52MOErG/*/i2MOErA/*/i2MOErT/*/i2MOErG/*/i2MOErC/*C*A*G*C*C*C*A*A*A*
G*/i2MOErG/*/i2MOErT/*/i2MOErG/*/i2MOErT/*/32MOErG/
PIKFYVE ASO57
TAGCTGAAAGCAACCTCTCC
/52MOErT/*/i2MOErA/*/i2MOErG/*/i2MOErC/*/i2MOErT/*G*A*A*A*G*C*A*A*C*
C*/i2MOErT/*/i2MOErC/*/i2MOErT/*/i2MOErC/*/32MOErC/
PIKFYVE ASO59
GCAACTGCTGGAGTAGTGCC
/52MOErG/*/i2MOErC/*/i2MOErA/*/i2MOErA/*/i2MOErC/*T*G*C*T*G*G*A*G*T*
A*/i2MOErG/*/i2MOErT/*/i2MOErG/*/i2MOErC/*/32MOErC/
PIKFYVE ASO61
AGTCACTATGGAGCAACTGC
/52MOErA/*/i2MOErG/*/i2MOErT/*/i2MOErC/*/i2MOErA/*C*T*A*T*G*G*A*G*C*
A*/i2MOErA/*/i2MOErC/*/i2MOErT/*/i2MOErG/*/32MOErC/
PIKFYVE ASO64
ATGATGTCCCTCCAAGATGA
/52MOErA/*/i2MOErT/*/i2MOErG/*/i2MOErA/*/i2MOErT/*G*T*C*C*C*T*C*C*A*
A*/i2MOErG/*/i2MOErA/*/i2MOErT/*/i2MOErG/*/32MOErA/
PIKFYVE ASO65
CTTTCCTGAAGCACAATAGG
/52MOErC/*/i2MOErT/*/i2MOErT/*/i2MOE*T/*/i2MOErC/*C*T*G*A*A*G*C*A*C*
A*/i2MOErA/*/i2MOErT/*/i2MOErA/*/i2MOErG/*/32MOErG/
PIKFYVE ASO66
CAATCCAAGGACTGACACAG
/52MOErC/*/i2MOE™A/*/i2MOErA/*/i2MOErT/*/i2MOErC/*C*A*A*G*G*A*C*T*G*
A*/i2MOErC/*/i2MOErA/*/i2MOErC/*/i2MOErA/*/32MOErG/
PIKFYVE ASO67
AACTGGTGTTACCTGTTTGC
/52MOErA/*/i2MOErA/*/i2MOErC/*/i2MOErT/*/i2MOErG/*G*T*G*T*T*A*C*C*T*
G*/i2MOErT/*/i2MOErT/*/i2MOErT/*/i2MOErG/*/32MOErC/
PIKFYVE ASO68
TTCTTCCATCTCTTTCTGTG
/52MOErT/*/i2MOErT/*/i2MOErC/*/i2MOErT/*/i2MOErT/*C*C*A*T*C*T*C*T*T*T
*/i2MOErC/*/i2MOErT/*/i2MOErG/*/i2MOErT/*/32MOErG/
PIKFYVE ASO69
GCAACCTGTTATTCCAAGCT
/52MOErG/*/i2MOErC/*/i2MOErA/*/i2MOErA/*/i2MOErC/*C*T*G*T*T*A*T*T*C*C
*/i2MOErA/*/i2MOErA/*/i2MOErG/*/i2MOErC/*/32MOErT/
PIKFYVE ASO70
CTGGATCAAAAGGAAATGGA
/52MOErC/*/i2MOErT/*/i2MOErG/*/i2MOErG/*/i2MOErA/*T*C*A*A*A*A*G*G*A*
A*/i2MOErA/*/i2MOErT/*/i2MOErG/*/i2MOErG/*/32MOErA/
PIKFYVE ASO71
TTACAACTGAGAGCAAAAGC
/52MOErT/*/i2MOErT/*/i2MOErA/*/i2MOErC/*/i2MOErA/*A*C*T*G*A*G*A*G*C*
A*/i2MOErA/*/i2MOErA/*/i2MOErA/*/i2MOErG/*/32MOErC/
PIKFYVE ASO72
GTACTATTTGTTGGAAGCCC
/52MOErG/*/i2MOErT/*/i2MOErA/*/i2MOErC/*/i2MOErT/*A*T*T*T*G*T*T*G*G*A
*/i2MOErA/*/i2MOErG/*/i2MOErC/*/i2MOErC/*/32MOErC/
PIKFYVE ASO73
ATTGAAGTTCCACATGAGGA
/52MOErA/*/i2MOErT/*/i2MOErT/*/i2MOErG/*/i2MOEA/*A*G*T*T*C*C*A*C*A*
T*/i2MOErG/*/i2MOErA/*/i2MOErG/*/i2MOErG/*/32MOErA/
PIKFYVE ASO74
AGCCACAGAACTGAAAAGCC
/52MOErA/*/i2MOErG/*/i2MOErC/*/i2MOErC/*/i2MOErA/*C*A*G*A*A*C*T*G*A*
A*/i2MOErA/*/i2MOErA/*/i2MOErG/*/i2MOErC/*/32MOErC/
PIKFYVE ASO75
TCATGCCACTTCCCAACCTA
/52MOErT/*/i2MOErC/*/i2MOErA/*/i2MOET/*/i2MOErG/*C*C*A*C*T*T*C*C*C*A
*/i2MOErA/*/i2MOErC/*/i2MOErC/*/i2MOErT/*/32MOErA/
PIKFYVE ASO76
GCAGCTCTACAAATGCTTTC
/52MOErG/*/i2MOErC/*/i2MOErA/*/i2MOErG/*/i2MOErC/*T*C*T*A*C*A*A*A*T*
G*/i2MOErC/*/i2MOErT/*/i2MOErT/*/i2MOErT/*/32MOErC/
PIKFYVE ASO77
CAGAACTAGCCATTATGTAG
/52MOErC/*/i2MOErA/*/i2MOErG/*/i2MOErA/*/i2MOErA/*C*T*A*G*C*C*A*T*T*
A*/i2MOErT/*/i2MOErG/*/i2MOET/*/i2MOErA/*/32MOErG/
PIKFYVE ASO78
GGTTTAGTAACTACAGACTC
/52MOErG/*/i2MOErG/*/i2MOErT/*/i2MOErT/*/i2MOErT/*A*G*T*A*A*C*T*A*C*
A*/i2MOErG/*/i2MOErA/*/i2MOErC/*/i2MOErT/*/32MOErC/
PIKFYVE ASO79
CACTCAATTTCTTGGATTGG
/52MOErC/*/i2MOErA/*/i2MOErC/*/i2MOErT/*/i2MOEC/*A*A*T*T*T*C*T*T*G*G
*/i2MOErA/*/i2MOErT/*/i2MOErT/*/i2MOErG/*/32MOErG/
PIKFYVE ASO80
GGTCTTGAACTCCTGACCTC
/52MOErG/*/i2MOErG/*/i2MOErT/*/i2MOErC/*/i2MOErT/*T*G*A*A*C*T*C*C*T*
G*/i2MOErA/*/i2MOErC/*/i2MOErC/*/i2MOErT/*/32MOErC/
PIKFYVE ASO81
GCTATTATTATAGGCAGTCC
/52MOErG/*/i2MOErC/*/i2MOErT/*/i2MOErA/*/i2MOErT/*T*A*T*T*A*T*A*G*G*
C*/i2MOErA/*/i2MOErG/*/i2MOErT/*/i2MOErC/*/32MOErC/
PIKFYVE ASO82
CCTCAGTGTTTTGTGTATCT
/52MOErC/*/i2MOErC/*/i2MOErT/*/i2MOErC/*/i2MOErA/*G*T*G*T*T*T*T*G*T*G
*/i2MOErT/*/i2MOErA/*/i2MOErT/*/i2MOErC/*/32MOErT/
PIKFYVE ASO83
TTATTGTCAGAAGCCATTCC
/52MOErT/*/i2MOErT/*/i2MOErA/*/i2MOErT/*/i2MOErT/*G*T*C*A*G*A*A*G*C*
C*/i2MOErA/*/i2MOErT/*/i2MOErT/*/i2MOErC/*/32MOErC/
PIKFYVE ASO84
CACAAAGTTACCTACTAGAC
/52MOErC/*/i2MOErA/*/i2MOErC/*/i2MOErA/*/i2MOErA/*A*G*T*T*A*C*C*T*A*
C*/i2MOErT/*/i2MOErA/*/i2MOErG/*/i2MOErA/*/32MOErC/
PIKFYVE ASO85
CTGACATAGTTTTCTCTTGC
/52MOErC/*/i2MOErT/*/i2MOErG/*/i2MOErA/*/i2MOErC/*A*T*A*G*T*T*T*T*C*T
*/i2MOErC/*/i2MOErT/*/i2MOErT/*/i2MOErG/*/32MOErC/
PIKFYVE ASO86
CATGAAGCTGATGACCACAA
/52MOErC/*/i2MOErA/*/i2MOErT/*/i2MOErG/*/i2MOErA/*A*G*C*T*G*A*T*G*A*
C*/i2MOErC/*/i2MOErA/*/i2MOErC/*/i2MOErA/*/32MOErA/
PIKFYVE ASO87
TTTCACTCTTCAAGCTCAGC
/52MOErT/*/i2MOErT/*/i2MOErT/*/i2MOErC/*/i2MOErA/*C*T*C*T*T*C*A*A*G*C
*/i2MOErT/*/i2MOErC/*/i2MOErA/*/i2MOErG/*/32MOErC/
PIKFYVE ASO88
CTTGAGGAATAATGAATGGC
/52MOErC/*/i2MOErT/*/i2MOErT/*/i2MOErG/*/i2MOErA/*G*G*A*A*T*A*A*T*G*
A*/i2MOErA/*/i2MOErT/*/i2MOErG/*/i2MOErG/*/32MOErC/
PIKFYVE ASO89
ATACACTGCACCTGTGTTAC
/52MOErA/*/i2MOErT/*/i2MOErA/*/i2MOErC/*/i2MOErA/*C*T*G*C*A*C*C*T*G*
T*/i2MOErG/*/i2MOErT/*/i2MOErT/*/i2MOErA/*/32MOErC/
PIKFYVE ASO90
CAAAACTGACACAGCAGTAT
/52MOErC/*/i2MOErA/*/i2MOErA/*/i2MOErA/*/i2MOErA/*C*T*G*A*C*A*C*A*G*
C*/i2MOErA/*/i2MOErG/*/i2MOErT/*/i2MOErA/*/32MOErT/
PIKFYVE ASO91
GCTAGAACCAAAGTAGAGAA
/52MOErG/*/i2MOErC/*/i2MOErT/*/i2MOErA/*/i2MOErG/*A*A*C*C*A*A*A*G*T*
A*/i2MOErG/*/i2MOErA/*/i2MOErG/*/i2MOErA/*/32MOErA/
PIKFYVE ASO92
GAGATGAGTAGAACTGGTGG
/52MOErG/*/i2MOErA/*/i2MOErG/*/i2MOErA/*/i2MOErT/*G*A*G*T*A*G*A*A*C*
T*/i2MOErG/*/i2MOErG/*/i2MOErT/*/i2MOErG/*/32MOErG/
PIKFYVE ASO93
CACTGCTGTCCAGAATCACT
/52MOErC/*/i2MOErA/*/i2MOErC/*/i2MOErT/*/i2MOErG/*C*T*G*T*C*C*A*G*A*
A*/i2MOErT/*/i2MOErC/*/i2MOErA/*/i2MOErC/*/32MOErT/
PIKFYVE ASO94
GGACTGTCTGAACAACCTGG
/52MOErG/*/i2MOErG/*/i2MOErA/*/i2MOErC/*/i2MOErT/*G*T*C*T*G*A*A*C*A*
A*/i2MOErC/*/i2MOErC/*/i2MOErT/*/i2MOErG/*/32MOErG/
PIKFYVE ASO95
GGACTGGAAACTGCTGACTG
/52MOErG/*/i2MOErG/*/i2MOErA/*/i2MOErC/*/i2MOErT/*G*G*A*A*A*C*T*G*C*
T*/i2MOErG/*/i2MOErA/*/i2MOErC/*/i2MOErT/*/32MOErG/
PIKFYVE ASO96
GGCTGCTACTCTTTGGTCTG
/52MOErG/*/i2MOErG/*/i2MOErC/*/i2MOErT/*/i2MOErG/*C*T*A*C*T*C*T*T*T*G
*/i2MOErG/*/i2MOErT/*/i2MOErC/*/i2MOErT/*/32MOErG/
PIKFYVE ASO97
CCTGGTAGTAAGCACTATCT
/52MOErC/*/i2MOErC/*/i2MOErT/*/i2MOErG/*/i2MOErG/*T*A*G*T*A*A*G*C*A*
C*/i2MOErT/*/i2MOErA/*/i2MOErT/*/i2MOErC/*/32MOErT/
MOUSE PIKFYVE ASOs for in vivo experiments
ASO gapmer (tested previously)
/52MOErG/*/i2MOErG/*/i2MOErT/*/i2MOErG/*/i2MOErC/*A*T*G*C*T*C*G*G*A*
G*/i2MOErG/*/i2MOErT/*/i2MOErC/*/i2MOErT/*/32MOErC/
Scrambled ASO
/52MOErG/*/i2MOErC/*/i2MOErG/*/i2MOErA/*/I2MOErC/T*A*T*A*C*G*C*G*C*
A*/i2MOErA/*/i2MOErT/*/i2MOErA/*/i2MOErT/*/32MOErG/

The PIKFYVE kinase antisense or inhibitory nucleic acids of the disclosure can inhibit the expression and thus the activity associated with PIKFYVE. The PIKFYVE kinase antisense or inhibitory nucleic acids can include any combination of the oligonucleotides set forth in Table 2 and sequences that are 98%-99% identical thereto.

Methods of treatment may include any number of modes of administering a disclosed composition or compound. Modes of administration may include aqueous, lipid, oily or other solutions, emulsions such as oil-in-water emulsions, liposomes, aqueous or oily suspensions and the like. Typically, an ASO of the disclosure will be administered directly to the CNS of the subject. Accordingly, the formulation or composition will be sterile and more preferably be suitable for injection. The following formulations and methods are merely exemplary and are in no way limiting.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The formulations may be presented in unit-dose or multi-dose sealed containers, such as ampules and vials, and may be stored in a freeze-dried (lyophilized) condition requiring only the addition of the sterile liquid excipient, for example, water, for injections, immediately prior to use. Extemporaneous injection solutions and suspensions may be prepared from sterile powders, granules, and tablets.

Additional therapeutic agent(s) may be administered simultaneously or sequentially with the disclosed one or more antisense or inhibitory nucleic acids and compositions. Sequential administration includes administration before or after the disclosed one or more antisense or inhibitory nucleic acids or compositions. In some embodiments, the additional therapeutic agent or agents may be administered in the same composition as the disclosed one or more antisense or inhibitory nucleic acids. In other embodiments, there may be an interval of time between administration of the additional therapeutic agent and the disclosed one or more antisense or inhibitory nucleic acids. In some embodiments, administration of an additional therapeutic agent with a disclosed one or more antisense or inhibitory nucleic acids may allow lower doses of the other therapeutic agents and/or administration at less frequent intervals. When used in combination with one or more other active ingredients, the one or more antisense or inhibitory nucleic acids of the disclosure and the other active ingredients may be used in lower doses than when each is used singly. Accordingly, the pharmaceutical compositions of the disclosure include those that contain one or more other active ingredients, in addition to one or more antisense or inhibitory nucleic acids of the disclosure. The above combinations include combinations of one or more antisense or inhibitory nucleic acids of the disclosure not only with one other active compound, but also with two or more other active compounds. For example, the compound of the disclosure may be combined with a variety of drugs to treat neurological diseases.

The disclosed one or more antisense or inhibitory nucleic acids can be combined with the following, but are not limited, anticholinergic drugs, anticonvulsants, antidepressants, benzodiazepines, decongestants, muscle relaxants, pain medications, and/or stimulants. Additional types of therapy and treatment include, but are not limited to digital communication devices, feeding tubes, mechanical ventilation, nutritional support, deep brain stimulation, occupational therapy, physical therapy, and/or speech therapy.

The disclosed composition(s) may be incorporated into a pharmaceutical composition suitable for administration to a subject (such as a patient, which may be a human or non-human). The pharmaceutical compositions may comprise a carrier (e.g., a pharmaceutically acceptable carrier). Any suitable carrier can be used within the context of the disclosure, and such carriers are well known in the art. The choice of carrier will be determined, in part, by the particular use of the composition (e.g., administration to an animal) and the particular method used to administer the composition. Accordingly, there is a wide variety of suitable formulations of the composition of the present invention.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of one or more antisense or inhibitory nucleic acids of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

The route by which the disclosed one or more compounds or compositions of the disclosure are administered and the form of the composition will dictate the type of carrier to be used.

The pharmaceutical compositions of the disclosure can be administered in a number of ways depending upon whether local or systemic treatment is desired and upon the area to be treated. Administration can be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In one embodiment the antisense or inhibitory nucleic acid is administered IV, IP, orally, topically or as a bolus injection or administered directly in to the target organ. In another embodiment, the antisense or inhibitory nucleic acid is administered intrathecal or intra-cerebroventricular as a bolus injection.

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The percentage of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The percentage of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The percentage of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The percentage of disintegrant(s) in a systemic composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The percentage of flavor(s), when used, in a systemic or topical composition is typically about 0.1 to about 1.0%.

Suitable antioxidants include butylated hydroxyanisole (“BHA”), butylated hydroxytoluene (“BHT”), and vitamin E. The percentage of antioxidant(s) in a systemic or topical composition is typically about 0.1 to about 5%.

Suitable preservatives include benzalkonium chloride, methyl paraben and sodium benzoate. The percentage of preservative(s) in a systemic or topical composition is typically about 0.01 to about 5%.

Suitable glidants include silicon dioxide. The percentage of glidant(s) in a systemic or topical composition is typically about 1 to about 5%.

Suitable solvents include water, isotonic saline, ethyl oleate, glycerin, hydroxylated castor oils, alcohols such as ethanol, and phosphate buffer solutions. The percentage of solvent(s) in a systemic or topical composition is typically from about 0 to about 100%.

Suitable suspending agents include AVICEL RC-591 (from FMC Corporation of Philadelphia, Pa.) and sodium alginate. The percentage of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The percentage of surfactant(s) in the systemic or topical composition is typically about 0.1% to about 5%.

Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients. For example, an intrathecal cerebrospinal fluid (CSF) catheter can be used to deliver antisense formulations of the disclosure. The catheter can be inserted at the L3 or L4 vertebrae. The distal tip of the catheter extends within the intrathecal space to approximately the L1 vertebrae. Antisense oligonucleotides are dissolved in saline, are sterilized by filtration, and are administered at 0.33 mL/min in a 1.0 mL volume followed by a 0.5 mL sterile water flush. Total infusion time is 4.5 min.

Although the amounts of components in the systemic compositions may vary depending on the type of systemic composition prepared, in general, systemic compositions include 0.01% to 50% of active compound and 50% to 99.99% of one or more carriers. Compositions for parenteral administration typically include 0.1% to 10% of actives and 90% to 99.9% of a carrier including a diluent and a solvent.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

In vivo testing of candidate antisense or inhibitory nucleic acids may be conducted by means known to one of ordinary skill in the art. For example, the candidate one or more antisense or inhibitory nucleic acids may be administered to a mammal, such as a mouse or a rabbit. The mammal may be administered, by any route deemed appropriate, a dose of a candidate antisense or inhibitory nucleic acids. Conventional methods and criteria can then be used to monitor animals for signs of reduction or improvement of motor neuron activity and/or expression or activity of PIKFYVE gene or protein, respectively. If needed, the results obtained in the presence of the candidate antisense or inhibitory nucleic acids can be compared with results in control animals that are not treated with the candidate antisense or inhibitory nucleic acids. Dosing studies may be performed in, or in conjunction with, the herein described methods for identifying one or more antisense or inhibitory nucleic acids capable of treating a neurological disease and/or any follow-on testing of candidate antisense or inhibitory nucleic acids in vivo. One of skill in the art of medicine may determine the appropriate dosage of one or more antisense or inhibitory nucleic acids. The dosage may be determined by monitoring the subject for signs of disease inhibition or amelioration. The dosage may be increased or decreased to obtain the desired frequency of treatment. The toxicity and efficacy of one or more antisense or inhibitory nucleic acids may be determined by standard pharmaceutical procedures in cell cultures or experimental animals, e.g. determining the lethal dose to 50% of the population (LD50) and the dose therapeutically effective in 50% of the population (ED50). The dose ratio of LD50/ED50 is the therapeutic index and, indicating the ratio between the toxic and therapeutic effects. A delivery system may be designed to help prevent toxic side effects, by delivering the one or more antisense or inhibitory nucleic acids to specific targets, e.g., delivered specifically to motor or central nervous system neurons. The optimal dose of the one or more antisense or inhibitory nucleic acids may be determined based on results of clinical electrophysiology or electromyography to analyze excitability in peripheral nerves, for example.

The dosage for use in humans may be determined by evaluating data obtained from animal studies and cell culture assays. The preferred dosage will have little or no toxicity and include the ED50. The dosage may vary depending on the dosage form and route of administration. For any antisense or inhibitory nucleic acid used in the methods described herein, the dosage may be estimated initially in cell culture. A dose may be formulated in animal models that includes the concentration of the test compound which achieves a half maximal inhibition of symptoms (LD50) as determined in cell culture. Such information obtained from cell cultures and animal models may be used to more accurately determine useful doses in humans.

The disclosure further provides that the methods and compositions described herein can be further defined by the following aspects (aspects 1 to 44):

1. A single stranded antisense oligonucleotide (ASO) that suppresses the expression of a PIKFYVE encoded by the sequence of SEQ ID NO:137, wherein the ASO comprises 12 to 50 linked nucleosides.

2. The ASO of aspect 1, wherein the ASO has 18 to 20 linked nucleosides.

3. The ASO of aspect 1 or aspect 2, wherein at least one internucleoside linkage is a modified internucleoside linkage.

4. The ASO of aspect 3, wherein at least one modified internucleoside linkage is a phosphorothioate internucleoside linkage.

5. The ASO of aspect 3, wherein each modified internucleoside linkage is a phosphorothioate internucleoside linkage.

6. The ASO of any one of the preceding aspects, wherein at least one internucleoside linkage is a phosphodiester internucleoside linkage.

7. The ASO of aspect 6, wherein at least one internucleoside linkage is a phosphorothioate linkage and at least one internucleoside linkage is a phosphodiester linkage.

8. The ASO of any one of the preceding aspects, wherein at least one nucleoside comprises a modified nucleobase.

9. The ASO of aspect 8, wherein the modified nucleobase is a 5-methylcytosine.

10. The ASO of any one of the preceding aspects, wherein at least one nucleoside of the ASO comprises a modified sugar moiety.

11. The ASO of aspect 10, wherein the at least one modified sugar moiety is a bicyclic sugar moiety.

12. The ASO of aspect 11, wherein the bicyclic sugar moiety comprises a 4′-CH(R)—O-2′ bridge wherein R is, independently, H, C1-12 alkyl, or a protecting group.

13. The ASO of aspect 12, wherein R is methyl.

14. The ASO of aspect 12, wherein R is H.

15. The ASO of aspect 10, wherein the modified sugar moiety comprises a 2′-O-methoxyethyl group.

16. The ASO of any one of the preceding aspects, where the ASO is a gapmer.

17. The ASO of aspect 16, wherein the ASO comprises:
a gap segment consisting of 8 to 12 linked deoxynucleosides;
a 5′ wing segment consisting of 3 to 5 linked nucleosides; and
a 3′ wing segment consisting of 3 to 5 linked nucleosides;
   wherein the gap segment is positioned between the 5′ wing segment and the 3′ wing segment and wherein a nucleoside of each wing segment comprises a modified sugar moiety.

18. The ASO of aspect 17, wherein each nucleoside of each wing segment comprises a modified sugar moiety.

19. The ASO of aspect 17, wherein the nucleosides making up each wing segment comprises at least two different modified sugar moieties.

20. The ASO of aspect 17, wherein the nucleosides making up each wing segment comprises the same modified sugar moiety.

21. The ASO of aspect 18, wherein the modified sugar moiety comprises a 2′-O-methoxyethyl group.

22. The ASO of any one of the preceding aspects, wherein the ASO has a nucleobase sequence that comprises at least 15 consecutive nucleobases of any of the nucleobase sequences of SEQ ID NOs: 1-136.

23. The ASO of any one of the preceding aspects, wherein the ASO has a nucleobase sequence of any one of SEQ ID NOs:1-136.

24. The ASO of aspect 23, wherein the ASO has a nucleobase sequence of any one of SEQ ID NOs:46, 49, 56, 60, 62, 64, 65, 70, 71, 73 and 105.

25. The ASO of any one of the preceding aspects, wherein the ASO is a gapmer consisting of a 5′ wing segment, a central gap segment, and a 3′ wing segment, wherein:
the 5′ wing segment consists of 3-5 modified nucleosides,
the central gap segment consists of 8-12 nucleosides, and the 3′ wing segment consists of 3-5 modified nucleosides;
wherein a modified nucleoside of each wing segment comprises a modified sugar moiety; and
wherein the ASO has the nucleobase sequence of any one of SEQ ID NOs: 1-136.

26. The ASO of aspect 25, wherein the ASO has a nucleobase sequence of any one of SEQ ID NOs:46, 49, 56, 60, 62, 64, 65, 70, 71, 73 and 105.

27. The ASO of aspect 25, wherein each modified nucleoside of each wing segment comprises a modified sugar moiety.

28. The ASO of aspect 27, wherein the modified nucleosides making up each wing segment comprises at least two different modified sugar moieties.

29. The ASO of aspect 27, wherein the modified nucleosides making up each wing segment comprises the same modified sugar moiety.

30. The ASO of aspect 27, wherein the modified sugar moiety comprises a 2′-O-methoxyethyl group.

31. A pharmaceutical composition comprising the ASO of any one of the preceding aspects, and a pharmaceutically acceptable carrier, diluent and/or excipient.

32. The pharmaceutical composition of aspect 31, wherein the pharmaceutical composition is formulated for parenteral delivery.

33. The pharmaceutical composition of aspect 31, wherein the pharmaceutical composition is formulated for intracerebroventricular injection.

34. A method of treating a subject having a neurological or neurodegenerative disease in need of treatment thereof, comprising:

administering a therapeutically effective amount of the pharmaceutical composition of any one of aspects 31 to 33, or a therapeutically effective amount of the ASO of any one of aspects 1 to 30.

35. The method of aspect 34, wherein the subject is haploinsufficient for the C9ORF72 gene.

36. The method of aspect 35, wherein the haploinsufficiency results in a 50% or greater reduction in C9ORF72 protein activity.

37. The method of aspect 36, wherein the C9ORF72 gene product comprises a dipeptide repeat resulting from the (GGGGCC)ₙ expansion.

38. The method of aspect 37, wherein the dipeptide repeat is cytotoxic.

39. The method of aspect 34, wherein the neurological disease is associated with neuronal hyperexcitability.

40. The method of aspect 34, wherein the neurological disease is associated with aberrant endosomal trafficking.

41. The method of aspect 34, wherein the neurological disease is associated with aberrant lysosomal trafficking.

42. The method of aspect 34, wherein the neurological disease is selected from the group consisting of familial and sporadic amyotrophic lateral sclerosis (ALS), familial and sporadic frontotemporal dementia (FTD), progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubro-pallidoluysian atrophy, spinocerebellar ataxias, and Creutzfeldt-Jakob disease.

43. The method of aspect 42, wherein the neurological disease is ALS.

44. The method of aspect 42, wherein the neurological disease is FTD.

The present invention has multiple aspects, illustrated by the following non-limiting examples.

### Examples

Identifying neurodegenerative processes caused by low C9ORF72 protein levels. To accurately determine how the C9ORF72 repeat expansion causes neurodegeneration, the mutation was studied in patient-derived neurons. Initial attempts to convert patient fibroblasts directly into iMNs using transcription factor overexpression were inefficient. It was found a major roadblock to conversion was that a build-up of genomic DNA torsion limited simultaneous high rates of transcription and replication, which is required for cellular reprogramming. Activating topoisomerases or using starting cells with high topoisomerase expression, such as iPSCs, enabled robust conversion into iMNs. Using this approach, it was found that reduced C9ORF72 levels leads to neurodegeneration, and that this occurs because iMNs become hypersensitive to C9ORF72 DPR and glutamate toxicity.

Determining the function of C9ORF72 protein. It was found that C9ORF72 is localized in intracellular vesicles in neurons, including early endosomes. Reduced C9ORF72 levels resulted in a depletion of lysosomes and impaired autophagosome formation. The lysosomal defects lead to an accumulation of glutamate receptors and enhanced glutamate-induced excitotoxicity in C9ORF72 iMNs. In addition, the autophagy defects impair clearance of C9ORF72 DPRs. PIKFYVE inhibition reduced glutamate receptor and DPR levels in C9ORF72 ALS/FTD iMNs by inducing secretory autophagy.

Patient-derived motor neurons recapitulate FTD processes in vitro. Genetic studies indicate that each FTD-linked gene account for only a small fraction of FTD patients and most cases do not have a known mutation. Thus, patient-specific FTD disease models were established to perform phenotypic chemical screens to identify therapeutic targets that rescue multiple forms of FTD.

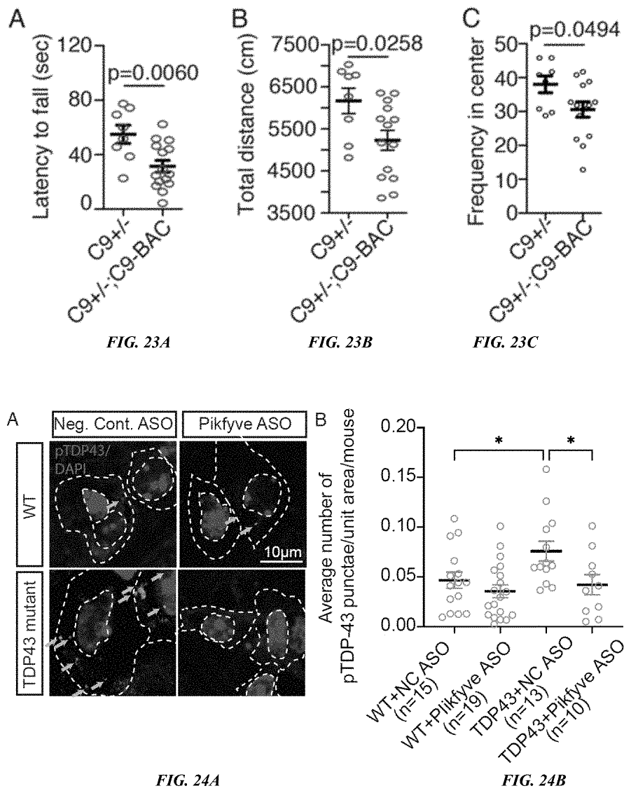

Mutations in MAPT cause FTD with tau aggregates. To determine if iNs from FTD patients display neurodegenerative phenotypes, an established method to generate cortical neurons from iPSCs by forced expression of NGN2 was used. 3 FTD patient iPSC lines harboring a MAPTV337M mutation and their CRISPR-edited isogenic controls from a published cell bank were utilized. Neurons from MAPT-mutant iPSCs display increased phosphorylated and oligomeric tau, similar to post-mortem patient tissue (FIG. 18A). Using longitudinal tracking of fluorescently-labeled iNs, MAPTV337M iNs were found to degenerate faster than controls (FIG. 18B). Thus, MAPT iNs display key features of FTD with tau pathology.

An intronic GGGGCC repeat expansion in C9ORF72 is the most common cause of FTD, accounting for 5-10% of cases. C9ORF72 patients develop DPR aggregates and nuclear loss of TDP-43, both of which can drive neurodegeneration. It was found that iNs from 6 C9ORF72 FTD patients degenerated faster than 6 controls (FIG. 18C, n=4 controls and 3 C9ORF72 patients shown). C9ORF72 iNs displayed DPR aggregates and TDP-43 mislocalization (FIG. 18D, E). Thus, C9ORF72 iNs provide a robust model of FTD.

iPSCs were established from 2 TARDBP FTD patients and the data suggests that iNs from these lines show similar neurodegenerative phenotypes to MAPT and C9ORF72 iNs. Thus, a collection of MAPT, C9ORF72, and TARDBP FTD lines that provide relevant models of FTD were established.

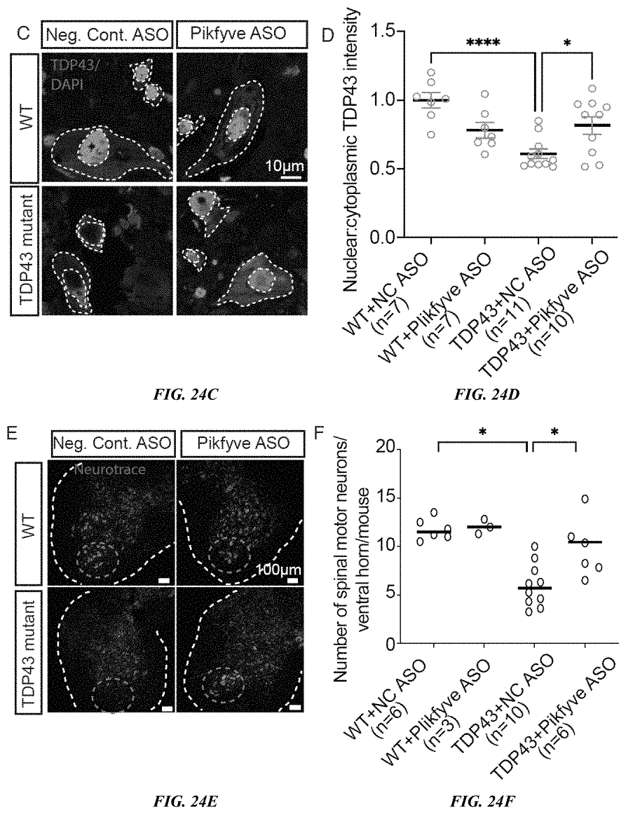

Identification of PIKFYVE inhibition as a new therapeutic target for multiple forms of FTD. 2000 approved drugs and 1800 target-annotated tool compounds were screened on C9ORF72 FTD iNs. A PIKFYVE kinase inhibitor was found to be the most potent rescuer of C9ORF72 iN survival. A structurally distinct PIKFYVE inhibitor, apilimod, and ASOs targeting PIKFYVE also rescued C9ORF72, but not control, iN survival, confirming that PIKFYVE is the target that mediates neuroprotection specifically in patient iNs (FIG. 19A, B). Critically, apilimod lowered DPR levels and rescued TDP-43 mislocalization in C9ORF72 iNs (FIG. 19C).

While most hits from the C9ORF72 iN screen were ineffective on MAPT and TARDBP iNs, apilimod potently rescued MAPT and TARDBP iNs (FIG. 19D, E). It was found that neurodegenerative effects of PIKFYVE inhibition published previously were due to off-target effects of the inhibitor used, YM201636. While YM201636 decreased control iN survival, apilimod and PIKFYVE ASOs did not. Thus, PIKFYVE inhibition rescues the survival of iNs from multiple different FTD patients.

Blocking PIKFYVE activity induces secretory autophagy. While the proteosome and autophagy are well-known systems for eliminating misfolded proteins, secretory autophagy, or exosome secretion, has recently been shown to maintain neuronal proteastasis in C. elegans and mice. In mice overexpressing TDP-43, neurons secrete exosomes containing phospho-TDP-43. These exosomes do not cause cytosolic aggregates of TDP-43 in primary neurons nor do they spread TDP-43 pathology in vivo. Blocking neuronal exosome secretion with a small molecule inhibitor of Neutral Sphingomyelinase 2 (GW4869) increased neuronal cytoplasmic TDP-43 aggregates, accelerated neurodegeneration, and decreased TDP-43 mouse survival. Thus, secretory autophagy maintains proteostasis and prevents neuronal death in TDP-43 mouse models.

PIKFYVE inhibition blocks autophagosome-lysosome fusion by preventing the conversion of phosphatidylinositol-3-phosphate (PI(3)P) to phosphatidylinositol-3,5-bisphosphate (PI(3,5)P2), altering the ratio of these phopholipids in lysosomal, autophagosomal, and endosomal membranes. One might expect this to be detrimental to neurons. However, inhibition of macroautophagy using PIKFYVE inhibition or bafilomycin activates secretory autophagy to jettison misfolded proteins from cells in TSG101+ exosomes enriched with autophagosome components such as LC3 and Optineurin (OPTN). Thus, PIKFYVE inhibition may rescue neurodegeneration by inducing exocytosis of misfolded proteins.

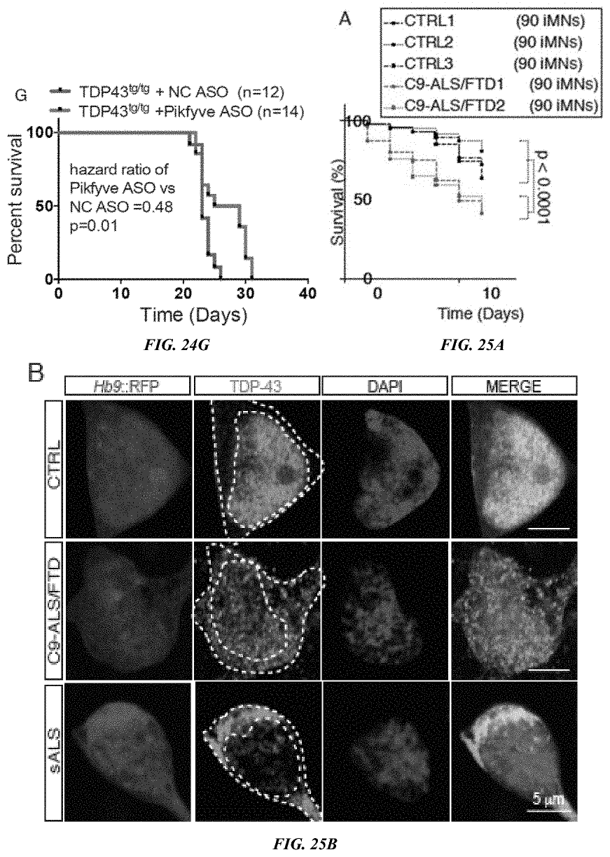

To test this, exosome release from iNs upon PIKFYVE inhibition was examined. Electron microscopy and western blotting confirmed the increased release of TSG101+ exosomes containing the neuronal marker Neurofilament heavy chain (FIG. 20A, B). Exosomes from apilimod-treated C9ORF72 FTD iNs contained high levels of TDP-43 (FIG. 20B, D), suggesting the secretion of cytoplasmic TDP-43 may explain the rescue of the nuclear:cytoplasmic TDP-43 ratio in C9ORF72 iNs upon PIKFYVE inhibition (FIG. 20C). FACS analysis of CFSE dye-labelled exosomes showed that iNs expressing a GFP-tagged form of C9ORF72 DPRs (poly(GR)50-GFP) released significantly more poly(GR)-GFP+ exosomes when treated with apilimod (FIG. 20E). Thus, PIKFYVE inhibition induces the secretion of TDP-43 and C9ORF72 DPRs from iNs

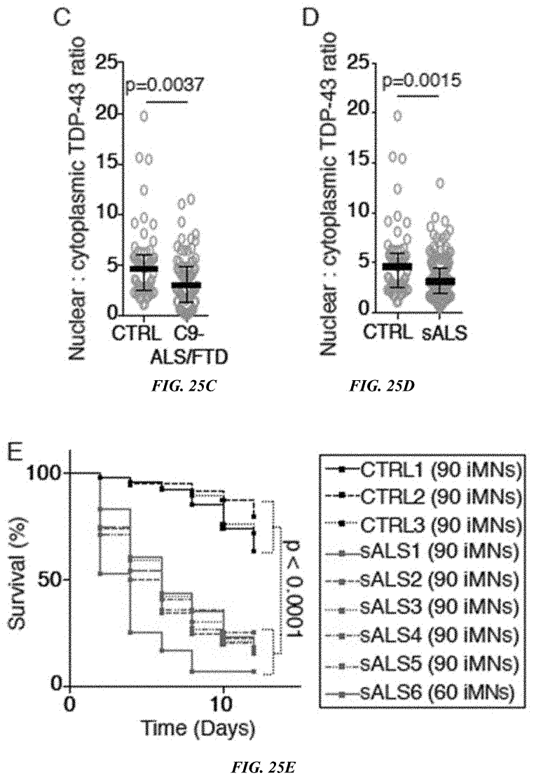

PIKFYVE inhibition rescues neurodegeneration through secretory autophagy. Consistent with a previous study in mice, GW4869 (inhibitor of Neutral Sphigomyelinase 2) treatment impaired apilimod-induced release of exosomes from C9ORF72 iNs (FIG. 20C, D). GW4869 blocked apilimod's ability to rescue C9ORF72 iN survival, suggesting that PIKFYVE inhibition rescues C9ORF72 FTD iN survival by inducing secretory autophagy (FIG. 21A, B).

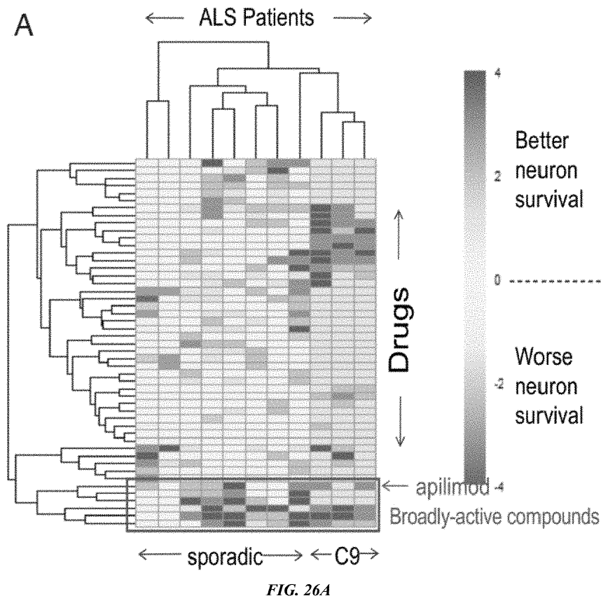

PIKFYVE inhibition rescues neurodegeneration in vivo. It was found that intracerebroventricular injection of apilimod into adult mice caused the release of OPTN into the cerebrospinal fluid (CSF)(18-fold increase in CSF OPTN levels by mass spectrometry, n=6 mice/group) and lowered DPR levels in C9ORF72-BAC mice, indicating that PIKFYVE inhibition induces secretory autophagy in vivo (FIG. 22A, B). Injection of a Pikfyve-suppressing ASO into the hippocampus of C9ORF72-BAC mice also lowered DPRs, confirming PIKFYVE as the relevant target (FIG. 22C). Since TARDBP overexpressing mice show motor deficits and neurodegeneration, we used TARDBP mice to test the efficacy of Pikfyve suppression. Intracerebroventricular injection of a Pikfyve ASO reduced Pikfyve mRNA levels and rescued motor deficits in TARDBP mice, indicating that reducing PIKFYVE activity rescues motor function in vivo (FIG. 22D, E). Importantly, GW4869 blocked motor rescue by PIKFYVE ASOs, confirming that PIKFYVE suppression rescues motor function by activating secretory autophagy (FIG. 22F).

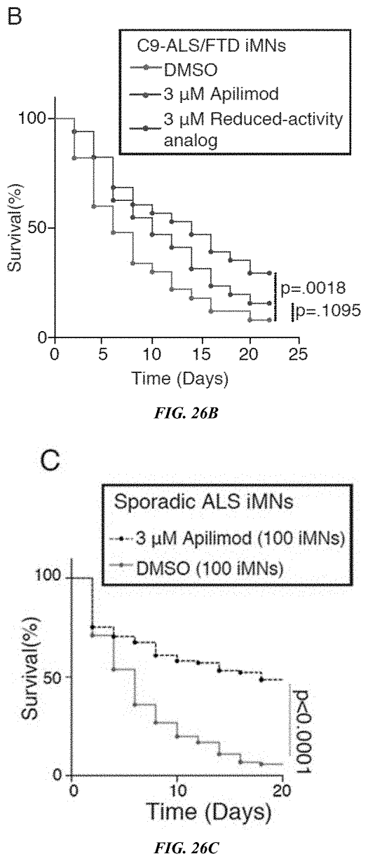

C9ORF72-BAC mice display cognitive and motor dysfunction. Mice harboring a repeat expanded C9ORF72 allele from an FTD/ALS patient display prominent DPRs and phosphorylated TDP-43 in cortical and spinal neurons. The C9ORF72 repeat expansion causes FTD and ALS through a combination of gain- and loss-of-function processes. While C9ORF72 loss-of-function mutations alone do not induce motor or cognitive dysfunction in mice, mice harboring both C9ORF72 loss- and gain-of-function mutations display behavioral phenotypes and neurodegeneration. The data confirm that male C9ORF72^{+/-}; C9ORF72-BAC mice display motor and cognitive defects compared to male C9ORF72^{+/-} mice at 9 months of age. Male C9ORF72^{+/-}; C9ORF72-BAC mice showed a reduced latency to fall in a hanging wire test and a decreased distance travelled over a 5 minute period in an open field test (FIG. 23A, B). C9ORF72^{+/-}; C9ORF72-BAC mice also displayed a reduced frequency in the center in an open field test, a sign of increased anxiety (FIG. 23C).

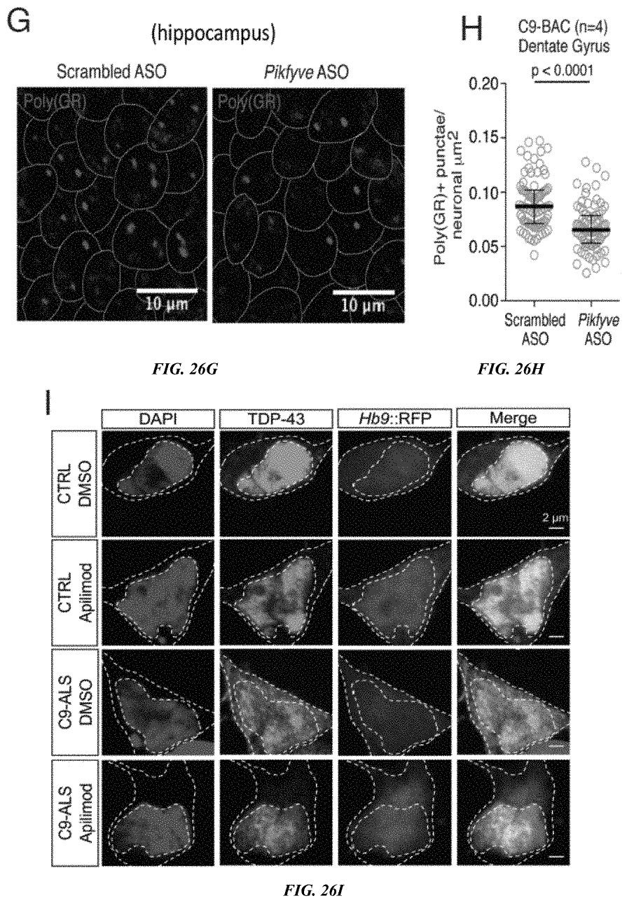

Patient-derived motor neurons recapitulate ALS processes in vitro. Genetic studies indicate that each ALS-linked gene is responsible for only a small fraction of ALS patients. In addition, over 80% of cases do not have a known causal mutation. Thus, a major focus is to identify therapeutic targets that rescue multiple forms of ALS. To this end, patient-specific ALS disease models were established and phenotypic screens using target-annotated small molecules was performed. A hexanucleotide repeat expansion in C9ORF72 is the most common cause of FTD and ALS, accounting for about 5-10% of each disease. C9ORF72 patients develop DPR aggregates and cytoplasmic mislocalization of TDP-43, both of which can drive neurodegeneration. Automated longitudinal imaging was used to show that iMNs generated from 6 C9ORF72 ALS/FTD patient iPSCs degenerate significantly faster than 6 controls (FIG. 25A, n=3 controls and 2 C9ORF72 patients shown). C9ORF72 patient iMNs produce DPRs and display DPR aggregates and TDP-43 misolocalization (FIG. 25B-D). Thus, C9ORF72 iMNs provided a robust model of C9ORF72 ALS/FTD. To determine if iMNs from sporadic ALS patients display neurodegenerative phenotypes, iMNs were generated from 8 sporadic ALS patients without known ALS mutations, all of which showed rapid degeneration and pronounced TDP-43 mislocalization compared to controls (FIG. 25E, 6/8 sporadic ALS lines shown). iMNs from different iPSC clones from the same patient survived similarly, ruling out clonal artifacts. Thus, sporadic ALS iPSC lines were identified that provide relevant models of sporadic ALS.

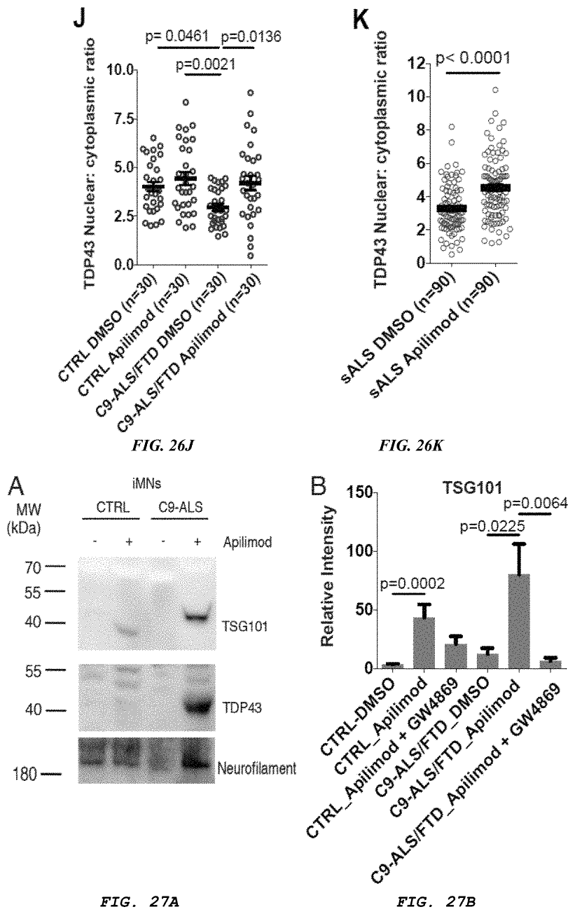

Drug screening iMNs from both C90RF72 and sporadic ALS patients. To identify new therapeutic targets for ALS, cellular reprogramming technology was used to generate induced motor neurons (iMNs) from both C9ORF72 and sporadic ALS patients. Chemical screens were then performed to search for targets that can rescue the degeneration of iMNs from multiple patients. 2000 approved drugs and 1800 target-annotated tool compounds were screened on C9ORF72 ALS/FTD iMNs. 40 compounds were identified that reproducibly rescued C9ORF72 iMN survival (FIG. 26A). Strikingly, when tested in 8 sporadic ALS iMNs, most hits were ineffective and/or differed greatly with respect to the particular sporadic ALS patient lines that responded to their treatment. However, 6 compounds rescued iMN survival in the majority of sporadic ALS patient lines, with the PIKFYVE kinase inhibitor apilimod being one of the most potent (FIG. 26B-C). A structurally distinct PIKFYVE inhibitor (YM201636) and multiple ASOs targeting PIKFYVE rescued C9ORF72 and sporadic ALS, but not control, iMN survival, confirming that PIKFYVE is the target that mediates neuroprotection specifically in patient iMNs (FIG. 26D-G). Neurodegenerative effects of PIKFYVE inhibition were likely due to off-target effects of the particular inhibitor used (YM201636): while YM201636 caused control iMN degeneration, apilimod and PIKFYVE ASOs did not. In addition, PIKFYVE ASOs lowered DPR levels in C9ORF72 BAC mice (FIG. 26H, I) and apilimod rescued the nuclear localization of TDP-43 in C9ORF72 and sporadic ALS iMNs (FIG. 26I-K). Thus, PIKFYVE is a rare target that can alter ALS disease processes in iMNs of multiple different ALS patients. Accordingly, inhibitors of PIKFYVE kinase were broadly-efficacious compounds across C9ORF72 and sporadic ALS iMNs.

Development of ASOs targeting the PIKFYVE gene. Small molecule inhibitors of PIKFYVE kinase and antisense oligonucleotides (ASOs) that suppress PIKFYVE expression can prevent the degeneration of human and mouse neurons that carry a mutation in the C9ORF72 gene that leads to amyotrophic lateral sclerosis (ALS) and frontotemporal dementia (FTD).

ASOs are an attractive therapeutic option for neurodegenerative diseases because of their ease of delivery to the central nervous system and their relatively low exposure to the periphery. These properties maximize target engagement in the central nervous system and minimize undesired target engagement or off-target effects in the periphery.

The disclosure provides novel antisense oligonucleotide (ASO) sequences targeting the PIKFYVE gene that can suppress PIKFYVE expression in human cells. PIKFYVE ASOs can also rescue the survival of motor neurons derived from sporadic ALS patients. Moreover, Pikfyve ASOs can lower levels of neurotoxic dipeptide repeat protein aggregates derived from the C9ORF72 repeat expansion in vivo in mice. To identify ASO sequences that suppress PIKFYVE expression in human cells, ASOs were designed (see, Table 2) and synthesized as MOE gapmers, which contains sugar and linkage modifications that increase nuclease resistance and melting temperature while maintaining the ability to be used as a substrate of RNase H.

Evaluating the ability of ASOs to suppress PIKFYVE RNA in vitro. The ability of each ASO to suppress PIKFYVE RNA levels was tested by transfecting them into human embryonic kidney 293T cells with Lipofectamine 2000 at a concentration of 100 nM, and measuring PIKFYVE expression 7 days after transfection. As a positive control, an HPRT-targeting ASO was used that has been validated to suppress HPRT expression. The relative PIKFYVE expression shown is an average of three technical replicates and values were calculated by normalizing to a GAPDH control. Together, these results show that several PIKFYVE ASOs suppress PIKFYVE expression in human cells.

Evaluating whether ASO-mediated PIKFYVE suppression can rescue the survival of C9ORF72 ALS/FTD and sporadic ALS patient-derived neurons. To determine if ASO-mediated PIKFYVE suppression can rescue the survival of C9ORF72 ALS/FTD and sporadic ALS patient-derived neurons, induced neurons were generated from C9ORF72 and sporadic ALS patients and treated them with scrambled or PIKFYVE ASOs. PIKFYVE inhibition can improve proteostasis in C9ORF72 ALS/FTD iMNs, resulting in reduced DPR and glutamate receptor levels. It was found that PIFKYVE inhibition induced secretory autophagy. Moreover, it was shown that sporadic ALS iMNs degenerated due to impaired proteostasis and glutamate receptor accumulation. By testing all hit compounds from the C9ORF72 iMN screen on sporadic ALS iMNs, it was found that most hit compounds were ineffective in sporadic ALS iMNs. However, PIKFYVE inhibition potently rescued sporadic ALS iMN survival. ASOs that suppress PIKFYVE also rescued ALS iMN survival. These results indicate that PIKFYVE ASOs can rescue the survival of sporadic ALS patient neurons.

Determining whether PIKfyve-targeting ASOs can reverse ALS disease progression in vivo. To determine if PIKfyve-targeting ASOs can reverse ALS disease processes in vivo, can PIKfyve ASOs lower levels of C9ORF72 dipeptide repeat proteins in mice. Dipeptide repeat proteins are generated from the C9ORF72 repeat expansion by repeat-associated non-AUG-dependent translation and are neurotoxic. C9ORF72 BAC mice harbor a BAC transgene containing a patient-derived, repeat expanded C9ORF72 allele and exhibit dipeptide repeat protein aggregates in their brain and spinal cord. Direct injection of PIKfyve ASO (Human PIKFYVE ASO Sequences Table 2) into the hippocampus significantly lowered PIKfyve expression and dipeptide repeat protein aggregate levels after 7 days. These results indicate that suppressing PIKfyve expression reverses ALS disease processes in vivo.

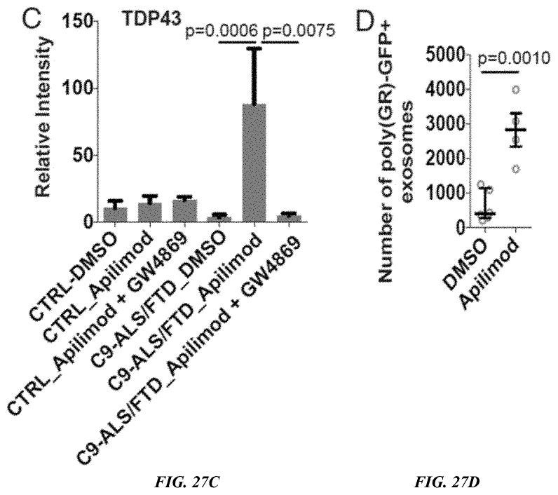

Blocking PIKFYVE activity induces secretory autophagy. While the proteosome and autophagy are well-known systems for eliminating misfolded or unwanted proteins, secretory autophagy, or exosome secretion, has recently been shown to maintain neuronal proteastasis in C. elegans and mice. In mice overexpressing TDP-43, neurons secrete exosomes containing phospho-TDP-43 that is pathogenic if transferred into immortalized cell lines; however, these exosomes are not taken up by primary neurons, suggesting that they do not spread pathogenic TDP-43 in vivo. Blocking neuronal exosome secretion with a small molecule inhibitor of Neutral Sphingomyelinase 2 (GW4869) or siRNA-mediated suppression of RAB27A increased neuronal accumulation of cytoplasmic TDP-43 aggregates, accelerated neurodegeneration, and decreased TDP-43 mouse survival. Thus, secretory autophagy maintains proteostasis and prevents neuronal death in TDP-43 mouse models. PIKFYVE inhibition blocks autophagosome-lysosome fusion by converting phosphatidylinositol-3-phosphate (PI(3)P) to phosphatidylinositol-3,5-bisphosphate (PI(3,5)P2) and thereby altering their ratio in endosomal, lysosomal and autophagosomal membranes. One might expect this to be detrimental to ALS iMNs, which can degenerate due to misfolded protein accumulation. However, inhibition of autophagosome-lysosome fusion using PIKFYVE inhibition or bafilomycin can activate secretory autophagy to jettison misfolded proteins from cells. Apilimod treatment causes secretion of TSG101+ exosomes enriched in autophagosomal proteins including p62, LC3, and OPTN, indicating that PIKFYVE inhibition may rescue neurodegeneration by inducing the exocytosis of misfolded proteins through secretory autophagy. To test this hypothesis, exosomal release in human iMNs upon PIKFYVE inhibition was examined. Western blotting showed that apilimod treatment significantly increased the release of TSG101+ exosomes containing the neuronal marker Neurofilament heavy chain (FIG. 27A, B). Exosomes from apilimod-treated C9ORF72 ALS iMNs contained high levels of TDP-43 (FIG. 27A, C), consistent with the rescue of the nuclear:cytoplasmic ratio of TDP-43 by secretion of cytoplasmic TDP-43 from C9ORF72 iMNs (FIG. 26I-K). FACS analysis of CFSE dye-labelled exosomes showed that iMNs expressing a GFP-tagged form of C9ORF72 DPRs (poly(GR)50-GFP) released significantly more poly(GR)-GFP+ exosomes when treated with apilimod (FIG. 27D). Thus, PIKFYVE inhibition induces the secretion of TDP-43 and C9ORF72 DPRs from iMNs.

Determining if PIKFYVE suppression rescues neurodegeneration through secretory autophagy. Consistent with a previous study in mice, GW4869 (inhibitor of Neutral Sphigomyelinase 2) treatment blocked apilimod-induced release of exosomes containing TSG101 and TDP-43 from ALS iMNs (FIG. 27B, C). ASOs were designed targeting RAB72A. It was confirmed that the ASOs suppressed RAB27A expression in iMNs.

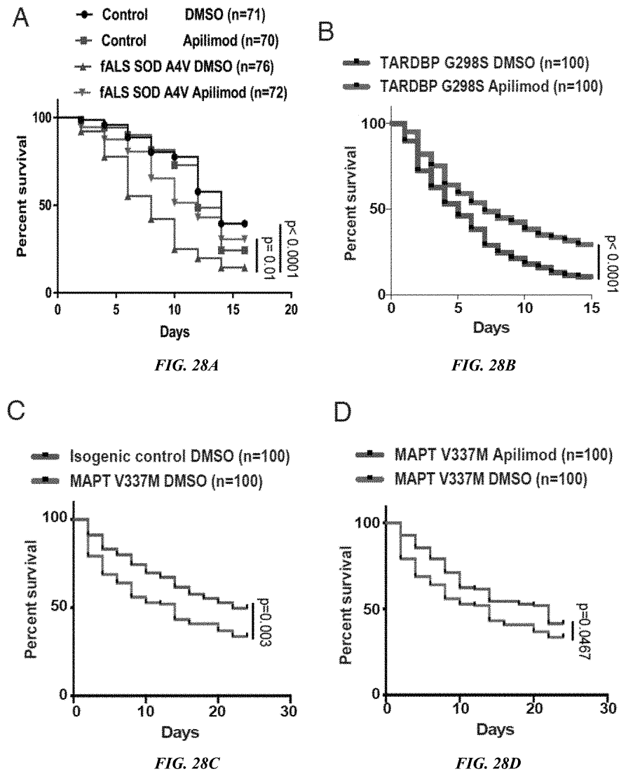

PIKFYVE inhibition rescues the survival of TARDBP ALS, SOD1 ALS, and MAPT FTD patient neurons. Since the accumulation of misfolded proteins is a common disease mechanism in all forms of ALS, FTD, and Alzheimer's disease, it was reasoned that inducing secretory autophagy might rescue neurodegeneration in diverse patients. iMNs were generated from ALS patients harboring mutations in TARDBP or SOD1 as well as NGN2-induced neurons (iNs) from MAPT^{V337M} FTD. Under neurotrophic factor withdrawal conditions, TARDBP and SOD1 ALS iMNs degenerated significantly faster than control iMNs, and MAPT FTD iNs degenerated significantly faster than iNs from an isogenic control line generated using CRISPR-Cas9 editing (FIG. 28A-C). As predicted, apilimod treatment rescued the survival of TARDBP ALS, SOD1 ALS, and MAPT FTD neurons (FIG. 28A, B, D). iPSC were generated from PSEN1^{A431E} early-onset Alzheimer's disease patients and CRISPR-Cas9 was used to generate isogenic control lines. PSEN1-mutant iNs degenerate significantly faster than isogenic control iNs (not shown due to space). 2 additional isogenic pairs of iPSC lines from MAPT FTD patients, 2 additional TARDBP ALS iPSC lines, and 2 additional SOD1 ALS iPSC lines were all obtained. These results suggest that PIKFYVE inhibition rescues neurodegeneration caused by diverse types of aggregation-prone proteins (TDP-43, SOD1, tau) establishing broad applicability across ALS, FTD, and Alzheimer's disease lines.

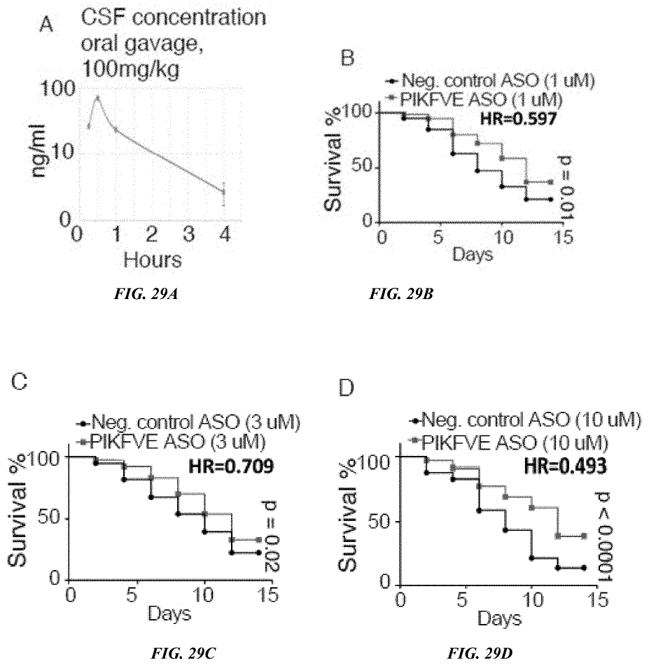

Testing the safety of ASO-mediated suppression of PIKFYVE as a therapeutic strategy. While apilimod is a potent and specific PIKFYVE inhibitor, it is not suitable for CNS indications because it has poor metabolic stability in vivo and does not achieve sufficient brain exposure to maintain its active concentration of 100 ng/ml (FIG. 29A). While it may be possible to generate a suitable PIKFYVE inhibitor using medicinal chemistry, this is an expensive and time-consuming endeavor. In contrast to small molecules, ASOs provide a facile approach to targeting the CNS because they can be injected directly into the spinal cord, achieve sustained target engagement throughout the CNS, and are less likely to cause peripheral toxicity. Thus, ASO-mediated PIKFYVE suppression was tested as a therapeutic approach for ALS, FTD, and Alzheimer's disease.

While humans and mice with one loss-of-function allele of PIKFYVE are healthy, complete loss of Pikfyve expression can cause toxicity in several organs. Experiments with PIKFYVE ASOs suggest that a 50% knockdown of PIKFYVE is sufficient to rescue ALS iMN survival. To test this more rigorously, CRISPR-Cas9 editing was used to introduce frameshift loss-of-function mutations into one allele of PIKFYVE in C9ORF72 ALS iPSCs. These cells ae then used to determine if a 50% reduction in PIKFYVE levels is sufficient to rescue neurodegeneration. To determine the therapeutic window of PIKFYVE ASOs in ALS patient iMNs, dose titrations of PIKFYVE ASOs were performed on both control and ALS iMNs. These studies show that PIKFYVE ASO administration of 1 uM strongly rescue ALS iMN survival (FIG. 29B) and that doses at least as high as 10 uM do not cause toxicity in iMNs (FIG. 29B-D). Thus, we have established tools and assays to determine the required level of PIKFYVE suppression and therapeutic window for rescuing neurodegeneration in iMNs.

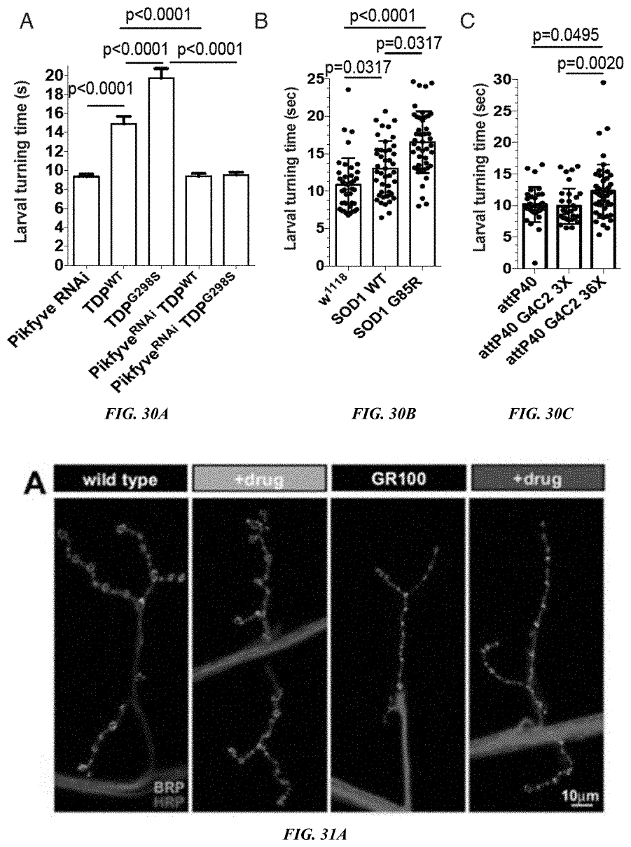

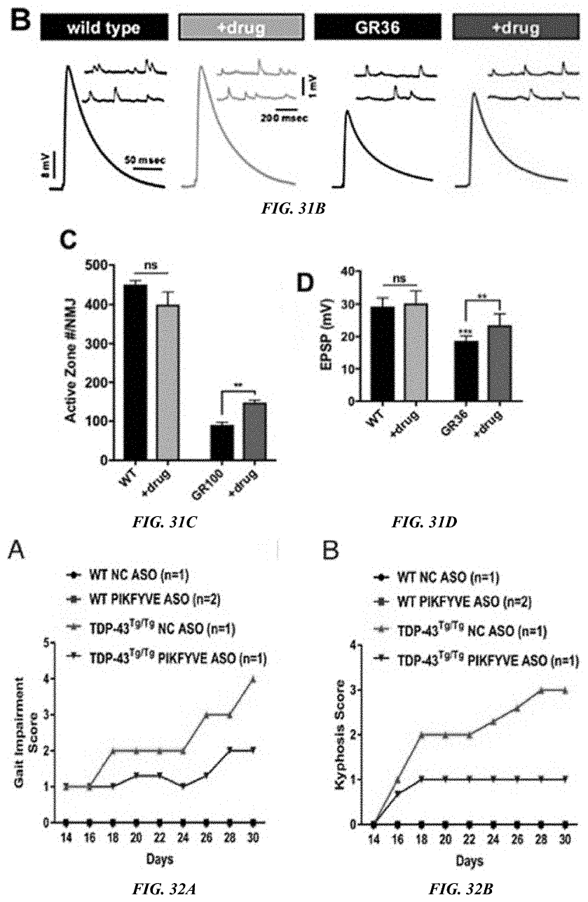

Testing the efficacy of PIKFYVE suppression in vivo. The fruit fly Drosophila is a powerful genetic system for studying the effects of ALS gene products on neuromuscular junction (NMJ) function. A Drosophila ALS model based on human TDP-43 expression has been developed that displays synaptic deficits, locomotor dysfunction, and reduced lifespan. It was shown that overexpression of wild-type or mutant TDP-43, SOD1, or the C9ORF72 repeat expansion in Drosophila larvae causes neurodegeneration and an increase in the time required for larvae to right themselves after being turned on their dorsal side, reflecting a decrease in motor function (FIG. 30A-C). Expression of an RNAi transgene targeting the Drosophila Pikfyve ortholog Fab1 potently rescues the larval turning time in TDP-43 overexpressing Drosophila (FIG. 30A), suggesting that Fab1/Pikfyve suppression rescues motor function. Overexpression of the C9ORF72 DPR poly(GR)(36 or 100 repeats) causes a dramatic reduction in synaptic arborization, active zone number, and synaptic strength at NMJs (FIG. 31A-D). Apilimod partially rescues the number of active zones and synaptic strength in poly(GR)-expressing larvae, and optimization of dosing is likely to yield larger improvements (FIG. 31A-D).

To determine if ASO-mediated Pikfyve suppression lowers C9ORF72 poly(GR) levels in mice, Pikfyve ASOs were injected into the hippocampus of adult C9ORF72-BAC mice, which harbor a C9ORF72 repeat expanded transgene and accumulate poly(GR)+ aggregates in hippocampal neurons. Pikfyve ASO treatment significantly reduced Pikfyve expression and poly(GR)+ aggregates by one week after injection (FIG. 26G, H). Injection of apilimod into the hippocampus of these mice also reduced levels of poly(PR) and poly(GR), indicating that PIKFYVE inhibition lowers levels of DPRs from sense and antisense C9ORF72 transcripts.

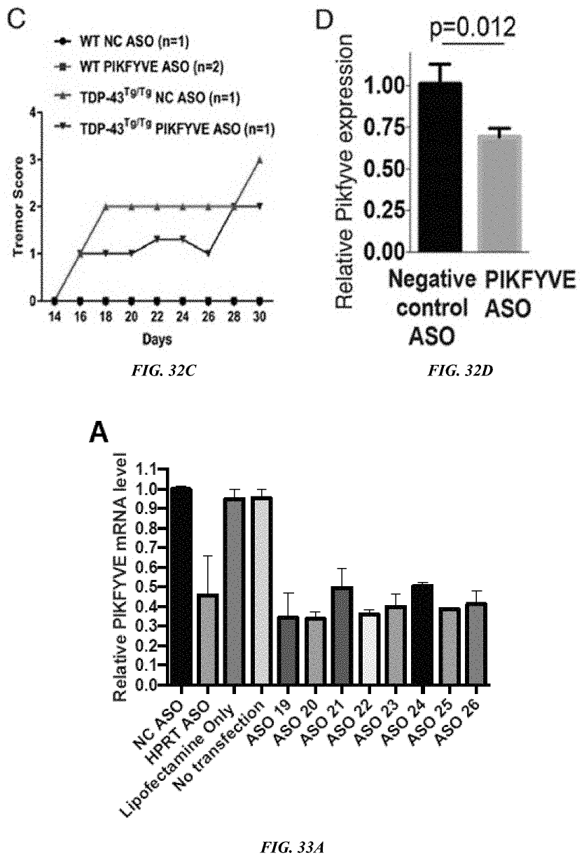

To determine if ASO-mediated PIKFYVE suppression can rescue neurodegeneration in mouse models of ALS, two mouse models—a C9ORF72 model based on adeno-associated virus (AAV)-mediated overexpression of poly(GR) and a TDP-43 mouse mode were established. Both models develop neurodegeneration, motor impairment, and paralysis. The data indicated that TDP-43 mice develop gait impairment, kyphosis, and tremor starting at about day 14 and reach paralysis by day 30 (FIG. 32A-C). To test the effect of Pikfyve suppression on neurodegeneration, ASOs were administered by intracerebroventricular injection at P0 as described. The Pikfyve-targeting ASO significantly decreased Pikfyve expression (FIG. 32D, n=5 mice per condition). The motor phenotypes and survival of TDP-43 mice injected with either a negative control or Pikfyve ASO were successfully tracked (FIG. 32A-C).

It will be understood that various modifications may be made without departing from the spirit and scope of this disclosure. Accordingly, other embodiments are within the scope of the following claims.

1. A single stranded antisense oligonucleotide (ASO) that suppresses the expression of a PIKFYVE encoded by the sequence of SEQ ID NO:137, wherein the ASO comprises 12 to 50 linked nucleosides.

2. (canceled)

3. The ASO of claim 1, wherein at least one internucleoside linkage is a modified internucleoside linkage.

4. The ASO of claim 3, wherein the at least one modified internucleoside linkage is a phosphorothioate internucleoside linkage.

5. (canceled)

6. The ASO of claim 1, wherein at least one internucleoside linkage is a phosphodiester internucleoside linkage.

7. The ASO of claim 6, wherein at least one internucleoside linkage is a phosphorothioate linkage and at least one internucleoside linkage is a phosphodiester linkage.

8. The ASO of claim 1, wherein at least one nucleoside comprises a modified nucleobase.

9. The ASO of claim 8, wherein the modified nucleobase is a 5-methylcytosine.

10. The ASO of claim 1, wherein at least one nucleoside of the ASO comprises a modified sugar moiety.

11. The ASO of claim 10, wherein the at least one modified sugar moiety is a bicyclic sugar moiety.

12. The ASO of claim 11, wherein the bicyclic sugar moiety comprises a 4′-CH(R)—O-2′ bridge wherein R is, independently, H, C₁₋₁₂ alkyl, or a protecting group.

13. The ASO of claim 12, wherein R is methyl or H.

14. (canceled)

15. The ASO of claim 10, wherein the modified sugar moiety comprises a 2′-O-methoxyethyl group.

16. The ASO of claim 1, where the ASO is a gapmer.

17. The ASO of claim 16, wherein the ASO comprises:
a gap segment consisting of 8 to 12 linked deoxynucleosides;
a 5′ wing segment consisting of 3 to 5 linked nucleosides; and
a 3′ wing segment consisting of 3 to 5 linked nucleosides;
wherein the gap segment is positioned between the 5′ wing segment and the 3′ wing segment and wherein a nucleoside of each wing segment comprises a modified sugar moiety.

18. The ASO of claim 17, wherein each nucleoside of each wing segment comprises a modified sugar moiety.

19. The ASO of claim 17, wherein the nucleosides making up each wing segment comprises at least two different modified sugar moieties.

20. The ASO of claim 17, wherein the nucleosides making up each wing segment comprises the same modified sugar moiety.

21. The ASO of claim 18, wherein the modified sugar moiety comprises a 2′-O-methoxyethyl group.

22. The ASO of claim 1, wherein the ASO has a nucleobase sequence that comprises at least 15 consecutive nucleobases of any of the nucleobase sequences of SEQ ID NOs: 1-136.

23. The ASO of claim 1, wherein the ASO has a nucleobase sequence of any one of SEQ ID NOs:1-136.

24. The ASO of claim 23, wherein the ASO has a nucleobase sequence of any one of SEQ ID NOs:46, 49, 56, 60, 62, 64, 65, 70, 71, 73 and 105.

25. The ASO of claim 1, wherein the ASO is a gapmer consisting of a 5′ wing segment, a central gap segment, and a 3′ wing segment, wherein:
the 5′ wing segment consists of 3-5 modified nucleosides,
the central gap segment consists of 8-12 nucleosides, and the 3′ wing segment consists of 3-5 modified nucleosides;
wherein a modified nucleoside of each wing segment comprises a modified sugar moiety; and
wherein the ASO has the nucleobase sequence of any one of SEQ ID NOs: 1-136.

26. The ASO of claim 25, wherein the ASO has a nucleobase sequence of any one of SEQ ID NOs:46, 49, 56, 60, 62, 64, 65, 70, 71, 73 and 105.

27. The ASO of claim 25, wherein each modified nucleoside of each wing segment comprises a modified sugar moiety.

28. The ASO of claim 27, wherein the modified nucleosides making up each wing segment comprises at least two different modified sugar moieties.

29. The ASO of claim 27, wherein the modified nucleosides making up each wing segment comprises the same modified sugar moiety.

30. The ASO of claim 27, wherein the modified sugar moiety comprises a 2′-O-methoxyethyl group.

31. A pharmaceutical composition comprising the ASO of claim 1, and a pharmaceutically acceptable carrier, diluent and/or excipient.

32. The pharmaceutical composition of claim 31, wherein the pharmaceutical composition is formulated for parenteral delivery.

33. The pharmaceutical composition of claim 31, wherein the pharmaceutical composition is formulated for intracerebroventricular injection.

34. A method of treating a subject having a neurological or neurodegenerative disease in need of treatment thereof, comprising:
administering a therapeutically effective amount of the pharmaceutical composition of claim 31.

35. The method of claim 34, wherein the subject is haploinsufficient for the C9ORF72 gene.

36. The method of claim 35, wherein the haploinsufficiency results in a 50% or greater reduction in C9ORF72 protein activity.

37. The method of claim 36, wherein the C9ORF72 gene product comprises a dipeptide repeat resulting from a (GGGGCC)ₙ expansion.

38. The method of claim 37, wherein the dipeptide repeat is cytotoxic.

39. The method of claim 34, wherein the neurological disease is associated with neuronal hyperexcitability.

40. The method of claim 34, wherein the neurological disease is associated with aberrant endosomal trafficking.

41. The method of claim 34, wherein the neurological disease is associated with aberrant lysosomal trafficking.

42. The method of claim 34, wherein the neurological disease is selected from the group consisting of familial and sporadic amyotrophic lateral sclerosis (ALS), familial and sporadic frontotemporal dementia (FTD), progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubro-pallidoluysian atrophy, spinocerebellar ataxias, Creutzfeldt-Jakob disease.

43-44. (canceled)


SEQUENCE LISTING (137 sequences)

SEQ ID NO: 1 - DNA, 20 bp, Artificial Sequence
  Antisense oligonucleotide
acttgtccattgcctcacaa

SEQ ID NO: 2 - DNA, 20 bp, Artificial Sequence
  Antisense oligonucleotide
tctcggtcccctccttgccc

SEQ ID NO: 3 - DNA, 20 bp, Artificial Sequence
  Antisense oligonucleotide
ccttcacttggctgtctgtg

SEQ ID NO: 4 - DNA, 20 bp, Artificial Sequence
  Antisense oligonucleotide
ctcatgccctcgtccctcaa

SEQ ID NO: 5 - DNA, 20 bp, Artificial Sequence
  Antisense oligonucleotide
tttccttcacttggctgtct

SEQ ID NO: 6 - DNA, 20 bp, Artificial Sequence
  Antisense oligonucleotide
ttgtctgtcctccactcatt

SEQ ID NO: 7 - DNA, 20 bp, Artificial Sequence
  Antisense oligonucleotide
tttcatgccacttcccaacc

SEQ ID NO: 8 - DNA, 20 bp, Artificial Sequence
  Antisense oligonucleotide
tgcccatttcggattagcca

SEQ ID NO: 9 - DNA, 20 bp, Artificial Sequence
  Antisense oligonucleotide
gctggtccaacttccactca

SEQ ID NO: 10 - DNA, 20 bp, Artificial Sequence
  Antisense oligonucleotide
catcctcagtggtctctgtc

SEQ ID NO: 11 - DNA, 20 bp, Artificial Sequence
  Antisense oligonucleotide
catgccctcgtccctcaatc

SEQ ID NO: 12 - DNA, 20 bp, Artificial Sequence
  Antisense oligonucleotide
atcctcagtggtctctgtcc

SEQ ID NO: 13 - DNA, 20 bp, Artificial Sequence
  Antisense oligonucleotide
cttccccttgtctcagtctt

SEQ ID NO: 14 - DNA, 20 bp, Artificial Sequence
  Antisense oligonucleotide
ttcgtaagcagctggtccat

SEQ ID NO: 15 - DNA, 20 bp, Artificial Sequence
  Antisense oligonucleotide
atgacttcaggtggcgtagg

SEQ ID NO: 16 - DNA, 20 bp, Artificial Sequence
  Antisense oligonucleotide
cttgattctcggtcccctcc

SEQ ID NO: 17 - DNA, 20 bp, Artificial Sequence
  Antisense oligonucleotide
gagccttgcttgcatcttct

SEQ ID NO: 18 - DNA, 20 bp, Artificial Sequence
  Antisense oligonucleotide
tggtcctttgttcctgtccc

SEQ ID NO: 19 - DNA, 20 bp, Artificial Sequence
  Antisense oligonucleotide
ttggtcctttgttcctgtcc

SEQ ID NO: 20 - DNA, 20 bp, Artificial Sequence
  Antisense oligonucleotide
ctccaactttagccctttga

SEQ ID NO: 21 - DNA, 20 bp, Artificial Sequence
  Antisense oligonucleotide
tgcctcagccacccaagtag

SEQ ID NO: 22 - DNA, 20 bp, Artificial Sequence
  Antisense oligonucleotide
gtgtaactgaacttgtccag

SEQ ID NO: 23 - DNA, 20 bp, Artificial Sequence
  Antisense oligonucleotide
gtcataagtccttggtcaac

SEQ ID NO: 24 - DNA, 20 bp, Artificial Sequence
  Antisense oligonucleotide
cttccagttcaaaacaaacc

SEQ ID NO: 25 - DNA, 20 bp, Artificial Sequence
  Antisense oligonucleotide
cctaaacaagagcagcaagg

SEQ ID NO: 26 - DNA, 20 bp, Artificial Sequence
  Antisense oligonucleotide
actcactgctaaatcatgga

SEQ ID NO: 27 - DNA, 20 bp, Artificial Sequence
  Antisense oligonucleotide
acagtccacctttgttgacc

SEQ ID NO: 28 - DNA, 20 bp, Artificial Sequence
  Antisense oligonucleotide
aaatgcccatcaaacacatc

SEQ ID NO: 29 - DNA, 20 bp, Artificial Sequence
  Antisense oligonucleotide
tctacttcatcttgaggctc

SEQ ID NO: 30 - DNA, 20 bp, Artificial Sequence
  Antisense oligonucleotide
ctgttgtcctccagtgtcag

SEQ ID NO: 31 - DNA, 20 bp, Artificial Sequence
  Antisense oligonucleotide
gatgccagcccaaaggtgtg

SEQ ID NO: 32 - DNA, 20 bp, Artificial Sequence
  Antisense oligonucleotide
tagctgaaagcaacctctcc

SEQ ID NO: 33 - DNA, 20 bp, Artificial Sequence
  Antisense oligonucleotide
gcaactgctggagtagtgcc

SEQ ID NO: 34 - DNA, 20 bp, Artificial Sequence
  Antisense oligonucleotide
agtcactatggagcaactgc

SEQ ID NO: 35 - DNA, 20 bp, Artificial Sequence
  Antisense oligonucleotide
atgatgtccctccaagatga

SEQ ID NO: 36 - DNA, 20 bp, Artificial Sequence
  Antisense oligonucleotide
ctttcctgaagcacaatagg

SEQ ID NO: 37 - DNA, 20 bp, Artificial Sequence
  Antisense oligonucleotide
caatccaaggactgacacag

SEQ ID NO: 38 - DNA, 20 bp, Artificial Sequence
  Antisense oligonucleotide
aactggtgttacctgtttgc

SEQ ID NO: 39 - DNA, 20 bp, Artificial Sequence
  Antisense oligonucleotide
ttcttccatctctttctgtg

SEQ ID NO: 40 - DNA, 20 bp, Artificial Sequence
  Antisense oligonucleotide
gcaacctgttattccaagct

SEQ ID NO: 41 - DNA, 20 bp, Artificial Sequence
  Antisense oligonucleotide
ctggatcaaaaggaaatgga

SEQ ID NO: 42 - DNA, 20 bp, Artificial Sequence
  Antisense oligonucleotide
ttacaactgagagcaaaagc

SEQ ID NO: 43 - DNA, 20 bp, Artificial Sequence
  Antisense oligonucleotide
gtactatttgttggaagccc

SEQ ID NO: 44 - DNA, 20 bp, Artificial Sequence
  Antisense oligonucleotide
attgaagttccacatgagga

SEQ ID NO: 45 - DNA, 20 bp, Artificial Sequence
  Antisense oligonucleotide
agccacagaactgaaaagcc

SEQ ID NO: 46 - DNA, 20 bp, Artificial Sequence
  Antisense oligonucleotide
tcatgccacttcccaaccta

SEQ ID NO: 47 - DNA, 20 bp, Artificial Sequence
  Antisense oligonucleotide
gcagctctacaaatgctttc

SEQ ID NO: 48 - DNA, 20 bp, Artificial Sequence
  Antisense oliognucleotide
cagaactagccattatgtag

SEQ ID NO: 49 - DNA, 20 bp, Artificial Sequence
  Antisense oligonucleotide
ggtttagtaactacagactc

SEQ ID NO: 50 - DNA, 20 bp, Artificial Sequence
  Antisense oligonucleotide
cactcaatttcttggattgg

SEQ ID NO: 51 - DNA, 20 bp, Artificial Sequence
  Antisense oligonucleotide
ggtcttgaactcctgacctc

SEQ ID NO: 52 - DNA, 20 bp, Artificial Sequence
  Antisense oligonucleotide
gctattattataggcagtcc

SEQ ID NO: 53 - DNA, 20 bp, Artificial Sequence
  Antisense oligonucleotide
cctcagtgttttgtgtatct

SEQ ID NO: 54 - DNA, 20 bp, Artificial Sequence
  Antisense oligonucleotide
ttattgtcagaagccattcc

SEQ ID NO: 55 - DNA, 20 bp, Artificial Sequence
  Antisense oligonucleotide
cacaaagttacctactagac

SEQ ID NO: 56 - DNA, 20 bp, Artificial Sequence
  Antisense oligonucleotide
ctgacatagttttctcttgc

SEQ ID NO: 57 - DNA, 20 bp, Artificial Sequence
  Antisense oligonucleotide
catgaagctgatgaccacaa

SEQ ID NO: 58 - DNA, 20 bp, Artificial Sequence
  Antisense oligonucleotide
tttcactcttcaagctcagc

SEQ ID NO: 59 - DNA, 20 bp, Artificial Sequence
  Antisense oligonucleotide
cttgaggaataatgaatggc

SEQ ID NO: 60 - DNA, 20 bp, Artificial Sequence
  Antisense oligonucleotide
atacactgcacctgtgttac

SEQ ID NO: 61 - DNA, 20 bp, Artificial Sequence
  Antisense oligonucleotide
caaaactgacacagcagtat

SEQ ID NO: 62 - DNA, 20 bp, Artificial Sequence
  Antisense oligonucleotide
gctagaaccaaagtagagaa

SEQ ID NO: 63 - DNA, 20 bp, Artificial Sequence
  Antisense oligonucleotide
gagatgagtagaactggtgg

SEQ ID NO: 64 - DNA, 20 bp, Artificial Sequence
  Antisense oligonucleotide
cactgctgtccagaatcact

SEQ ID NO: 65 - DNA, 20 bp, Artificial Sequence
  Antisense oligonucleotide
ggactgtctgaacaacctgg

SEQ ID NO: 66 - DNA, 20 bp, Artificial Sequence
  Antisense oligonucleotide
ggactggaaactgctgactg

SEQ ID NO: 67 - DNA, 20 bp, Artificial Sequence
  Antisense oligonucleotide
ggctgctactctttggtctg

SEQ ID NO: 68 - DNA, 20 bp, Artificial Sequence
  Antisense oligonucleotide
cctggtagtaagcactatct

SEQ ID NO: 69 - DNA, 20 bp, Artificial Sequence
  Antisense oligonucleotide
aacaggcaagctggaaagat

SEQ ID NO: 70 - DNA, 20 bp, Artificial Sequence
  Antisense oligonucleotide
gccaggatagtcaatttatg

SEQ ID NO: 71 - DNA, 20 bp, Artificial Sequence
  Antisense oligonucleotide
ccttggcagttttacacagg

SEQ ID NO: 72 - DNA, 20 bp, Artificial Sequence
  Antisense oligonucleotide
tctccaaaagttcaaggcag

SEQ ID NO: 73 - DNA, 20 bp, Artificial Sequence
  Antisense oligonucleotide
ggttacaggtatatgccacc

SEQ ID NO: 74 - DNA, 20 bp, Artificial Sequence
  Antisense oligonucleotide
ccaggttcaaacaattctcc

SEQ ID NO: 75 - DNA, 20 bp, Artificial Sequence
  Antisense oligonucleotide
tcttgctctgttgcccaggc

SEQ ID NO: 76 - DNA, 20 bp, Artificial Sequence
  Antisense oligonucleotide
atggatggagtgaccacagg

SEQ ID NO: 77 - DNA, 20 bp, Artificial Sequence
  Antisense oligonucleotide
aatgacatagaccaggactc

SEQ ID NO: 78 - DNA, 20 bp, Artificial Sequence
  Antisense oligonucleotide
ggaacttagcatcacattag

SEQ ID NO: 79 - DNA, 20 bp, Artificial Sequence
  Antisense oligonucleotide
ggagtggaacacatacttca

SEQ ID NO: 80 - DNA, 20 bp, Artificial Sequence
  Antisense oligonucleotide
gcaagtctttcatcaatggc

SEQ ID NO: 81 - DNA, 20 bp, Artificial Sequence
  Antisense oligonucleotide
cagagactttgtttcttggc

SEQ ID NO: 82 - DNA, 20 bp, Artificial Sequence
  Antisense oligonucleotide
ttctccattctgaagtcctg

SEQ ID NO: 83 - DNA, 20 bp, Artificial Sequence
  Antisense oligonucleotide
ctctggctggacaaagttgt

SEQ ID NO: 84 - DNA, 20 bp, Artificial Sequence
  Antisense oligonucleotide
ggacaacattccagaagcct

SEQ ID NO: 85 - DNA, 20 bp, Artificial Sequence
  Antisense oligonucleotide
cagtaaaacttggcattagc

SEQ ID NO: 86 - DNA, 20 bp, Artificial Sequence
  Antisense oligonucleotide
cagtgttgttctgagagttc

SEQ ID NO: 87 - DNA, 20 bp, Artificial Sequence
  Antisense oligonucleotide
aagacatgagccttgcttgc

SEQ ID NO: 88 - DNA, 20 bp, Artificial Sequence
  Antisense oligonucleotide
tggaacacttcaacagaaga

SEQ ID NO: 89 - DNA, 20 bp, Artificial Sequence
  Antisense oligonucleotide
taggcaactgaaatatctgc

SEQ ID NO: 90 - DNA, 20 bp, Artificial Sequence
  Antisense oligonucleotide
acatcactaaatcaccttgg

SEQ ID NO: 91 - DNA, 20 bp, Artificial Sequence
  Antisense oligonucleotide
atttgtgacaagtgcccagg

SEQ ID NO: 92 - DNA, 20 bp, Artificial Sequence
  Antisense oligonucleotide
ggtgctgtggacaaccttca

SEQ ID NO: 93 - DNA, 20 bp, Artificial Sequence
  Antisense oligonucleotide
tcataatcagagcctcctct

SEQ ID NO: 94 - DNA, 20 bp, Artificial Sequence
  Antisense oligonucleotide
gagaatgataagcaacacag

SEQ ID NO: 95 - DNA, 20 bp, Artificial Sequence
  Antisense oligonucleotide
aggcatagcaaattcatcca

SEQ ID NO: 96 - DNA, 20 bp, Artificial Sequence
  Antisense oligonucleotide
ttcctgctcacccttctcaa

SEQ ID NO: 97 - DNA, 20 bp, Artificial Sequence
  Antisense oligonucleotide
caaactgctatcatcacagg

SEQ ID NO: 98 - DNA, 20 bp, Artificial Sequence
  Antisense oligonucleotide
tgcttcacagaggcaacagc

SEQ ID NO: 99 - DNA, 20 bp, Artificial Sequence
  Antisense oligonucleotide
tcttgttggtcatccacagg

SEQ ID NO: 100 - DNA, 20 bp, Artificial Sequence
  Antisense oligonucleotide
caggacttggaggaactgaa

SEQ ID NO: 101 - DNA, 20 bp, Artificial Sequence
  Antisense oligonucleotide
ctggctgtatctagctcagg

SEQ ID NO: 102 - DNA, 20 bp, Artificial sequence
  Antisense oligonucleotide
caaagatggctttcatggtt

SEQ ID NO: 103 - DNA, 20 bp, Artificial Sequence
  Antisense oligonucleotide
tataatccagtgtcatcctg

SEQ ID NO: 104 - DNA, 20 bp, Artificial Sequence
  Antisense oligonucleotide
gagatacagaggatcacatc

SEQ ID NO: 105 - DNA, 20 bp, Artificial Sequence
  Antisense oligonucleotide
agaccagtaaacctgctctg

SEQ ID NO: 106 - DNA, 20 bp, Artificial Sequence
  Antisense oligonucleotide
catgccctgaagtccagaga

SEQ ID NO: 107 - DNA, 20 bp, Artificial Sequence
  Antisense oligonucleotide
cagaagagctgctgaagagc

SEQ ID NO: 108 - DNA, 20 bp, Artificial Sequence
  Antisense oligonucleotide
gcctgaaacagtatctctct

SEQ ID NO: 109 - DNA, 20 bp, Artificial Sequence
  Antisense oligonucleotide
gcagctctttggatttttcc

SEQ ID NO: 110 - DNA, 20 bp, Artificial Sequence
  Antisense oligonucleotide
acctggcagaccaatgacac

SEQ ID NO: 111 - DNA, 20 bp, Artificial Sequence
  Antisense oligonucleotide
catcatcctggttcttgaca

SEQ ID NO: 112 - DNA, 20 bp, Artificial Sequence
  Antisense oligonucleotide
caaacaaagccattgacaac

SEQ ID NO: 113 - DNA, 20 bp, Artificial Sequence
  Antisense oligonucleotide
tctacccaagctctggctat

SEQ ID NO: 114 - DNA, 20 bp, Artificial Sequence
  Antisense oligonucleotide
cagaacatgcttggacactg

SEQ ID NO: 115 - DNA, 20 bp, Artificial Sequence
  Antisense oligonucleotide
atccaactccttcaggatta

SEQ ID NO: 116 - DNA, 20 bp, Artificial Sequence
  Antisense oligonucleotide
gcaatgagtgactcaaagac

SEQ ID NO: 117 - DNA, 20 bp, Artificial Sequence
  Antisense oligonucleotide
cagacatgacttcaggtggc

SEQ ID NO: 118 - DNA, 20 bp, Artificial Sequence
  Antisense oligonucleotide
tttccctgctgtccctctga

SEQ ID NO: 119 - DNA, 20 bp, Artificial Sequence
  Antisense oligonucleotide
gaaggcagctcctgatttgc

SEQ ID NO: 120 - DNA, 20 bp, Artificial Sequence
  Antisense oligonucleotide
ggagattttcatctagcagg

SEQ ID NO: 121 - DNA, 20 bp, Artificial Sequence
  Antisense oligonucleotide
ctataatgaggtggctagaa

SEQ ID NO: 122 - DNA, 20 bp, Artificial Sequence
  Antisense oligonucleotide
ccaactactagctcattgct

SEQ ID NO: 123 - DNA, 20 bp, Artificial Sequence
  Antisense oligonucleotide
ctcacaaaacctagtcctgt

SEQ ID NO: 124 - DNA, 20 bp, Artificial Sequence
  Antisense oligonucleotide
tttcagcaattcagacccaa

SEQ ID NO: 125 - DNA, 20 bp, Artificial Sequence
  Antisense oligonucleotide
gacagctccacaggtatcaa

SEQ ID NO: 126 - DNA, 20 bp, Artificial Sequence
  Antisense oligonucleotide
aacaaattcagcagctctac

SEQ ID NO: 127 - DNA, 20 bp, Artificial Sequence
  Antisense oligonucleotide
ataggcagtccaaaaacagt

SEQ ID NO: 128 - DNA, 20 bp, Artificial Sequence
  Antisense oligonucleotide
tttgccacaaagttacctac

SEQ ID NO: 129 - DNA, 20 bp, Artificial Sequence
  Antisense oligonucleotide
gcacctgtgttaccaaaaaa

SEQ ID NO: 130 - DNA, 20 bp, Artificial Sequence
  Antisense oligonucleotide
aagtctccaaaagttcaagg

SEQ ID NO: 131 - DNA, 20 bp, Artificial Sequence
  Antisense oligonucleotide
gtcacttattgagagctgtt

SEQ ID NO: 132 - DNA, 20 bp, Artificial Sequence
  Antisense oligonucleotide
tttgatgtggacaaactgac

SEQ ID NO: 133 - DNA, 20 bp, Artificial Sequence
  Antisense oligonucleotide
gtagagatactcaatggaac

SEQ ID NO: 134 - DNA, 20 bp, Artificial Sequence
  Antisense oligonucleotide
tccagcaaactgctatcatc

SEQ ID NO: 135 - DNA, 20 bp, Artificial Sequence
  Antisense oligonucleotide
tcacccttctcaaacacaat

SEQ ID NO: 136 - DNA, 20 bp, Artificial Sequence
  Antisense oligonucleotide
tggcctccttctgctctctc

SEQ ID NO: 137 - DNA, 6522 bp, Homo sapiens
gtcgcaaccatgtaagcagcttcgcttcctgccgcaaccgtccgcggcctgaggagcccaccgccgctctcgggggccgacttccgggggctgagccgttgaagcggaggctggggcggggggcagccggcgcggccggggcaggaggcgcagactcatgaaatggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaagagtatttgatttctgacactggaggacaacagctctcaataagtgacgctttcatcaaagaatccttatttaatcgccgagtagaggaaaaatccaaagagctgcctttcacacctttgggctggcatcataacaacctggagctcctgagggaggagaatggggagaaacaagccatggagaggttgctttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaaatcccaggtggaaagaagtttgattctgtggttgtcaatggctttgtttgtaccaagaacattgcacataaaaagatgaattcttgtattaaaaaccctaaaattcttctgttgaagtgttccattgagtatctctacagagaagaaactaagtttacttgcattgatcctattgtgcttcaggaaagggaattcttgaagaattatgtccagcgaatagttgatgttcgacccaccttggttcttgttgagaaaacagtgtctcggattgcccaggacatgttattggaacatggcattactttggtcattaatgtaaagtcacaagttttggaacgaatcagtcgaatgacccaaggtgatttagtgatgtcaatggaccagctgcttacgaaaccacacctgggcacttgtcacaaattttatatgcagatatttcagttgcctaatgaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaatcgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagctgccagagagtttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggcaaatcaggaagcaaaaatgagggtgatgaagagagagggcttattctgagtgatgctgtgtggtcaacaaaggtggactgtctgaatcccattaatcaccagagactttgtgtgctcttcagcagctcttctgcccagtccagcaatgctcctagtgcctgtgtcagtccttggattgtaacaatggaattttatggaaagaatgatcttacattaggaatatttttagagagatactgtttcaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcagttattctcccattcggcttcttgaagtatgtgttccactccccaaaatattcattaagcgtcaggccccattaaaagtgtcccttcttcaggatctgaaggacttctttcaaaaagtttcacaggtatatgttgccattgatgaaagacttgcatctttgaaaactgatacatttagtaaaacaagagaggaaaaaatggaagatatttttgcacagaaagagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggttgcaggaccttttccaacaggaaaagggtagaaagagaccttcagttcctccaagtcctggaagactgagacaaggggaagaaagcaagataagtgcgatggatgcatctccacggaatatttctccaggacttcagaatggagaaaaagaggatcgcttcttaacaactttgtccagccagagctccaccagttctactcatctccaattgcctacgccacctgaagtcatgtctgaacagtcagtgggagggccccctgagctagatacagccagcagttccgaagatgtgtttgatgggcatttgctgggatccacagacagccaagtgaaggaaaagtcaaccatgaaagccatctttgcaaatttgcttccaggaaatagctataatcctattccatttccttttgatccagataaacactacttaatgtatgaacatgaacgagtgcccattgcagtctgcgagaaggaacccagctccatcattgcttttgctctcagttgtaaagaataccgaaatgccttagaggaattgtctaaagcgactcagtggaacagtgccgaagaagggcttccaacaaatagtacttcagatagcagaccaaagagtagcagccctatcagattacctgaaatgagtggaggacagacaaatcgtacaacagaaacagaaccacaaccaaccaaaaaggcttctggaatgttgtccttcttcagagggacagcagggaaaagccccgatctctcttcccagaagagagagaccttacgtggagcagatagtgcttactaccaggttgggcagacgggcaaggaggggaccgagaatcaaggcgttgagcctcaagatgaagtagatggaggagatacacaaaagaagcaactcataaatcctcatgtggaacttcaattttcagatgctaatgccaagttttactgtcggctctactatgcgggagagtttcataagatgcgtgaagtgattctggacagcagtgaagaagatttcattcgttccctctcccactcatcaccctggcaggcccggggaggcaaatcaggagctgccttctatgcaactgaggatgatagatttattttgaagcaaatgcctcgtctggaagtccagtccttcctcgactttgcaccacattacttcaattatattacaaatgctgttcaacaaaagaggcccacggcgttggccaaaattcttggagtttacagaattggttataagaactctcagaacaacactgagaagaagttagatctccttgtcatggaaaatcttttctacgggagaaagatggcacaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattatagattatattcgaacatttacatgggacaaaaagcttgagatggttgtgaaatcaacaggaattttaggtggacaaggtaaaatgccaacagtggtgtctccggagttgtacaggactaggttttgtgaggcaatggacaagtatttcctaatggtaccagaccactggacaggcttgggtctgaattgctgaaatcaagcacatattttgaaatggactgtgaaggaaaaggggacaggaacaaaggaccaaaaa